(19) United States
(12) Patent Application Publication
(10) Pub. No.: US 2008/0171756 A1
(43) Pub. Date: Jul. 17, 2008

(54) N-Substituted Glycine Derivatives: Prolyl Hydroxylase Inhibitors

(76) Inventors: Antony N. Shaw, Collegeville, PA (US); Kevin J. Duffy, Collegeville, PA (US); Rosanna Tedesco, Collegeville, PA (US); Kenneth Wiggall, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/972,707

(22) Filed: Jan. 11, 2008

Related U.S. Application Data
(60) Provisional application No. 60/884,706, filed on Jan. 12, 2007.

(51) Int. Cl. C07D 239/54; A61K 31/513; A61P 7/06

(52) U.S. Cl. 514/256; 544/319

(57) ABSTRACT

The invention described herein relates to certain pyrimidinedione N-substituted glycine derivatives of formula (I)

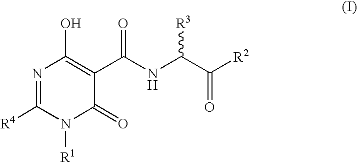

which are antagonists of HIF prolyl hydroxylases and are useful for treating diseases benefiting from the inhibition of this enzyme, anemia being one example.

### FIELD OF THE INVENTION

This invention relates to certain heteroaromatic N-substituted glycine derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1alpha, HIF-2alpha, and HIF-3alpha) are rapidly degraded by proteosome under normoxic conditions upon hydroxylation of proline residues by prolyl hydroxylases (EGLN1,2,3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

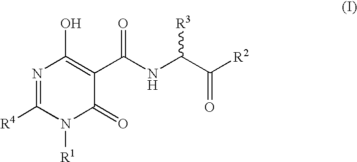

wherein:

R¹ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R² is —NR⁷R⁸ or —OR⁹;

R³ is H or C₁₋C₄alkyl;

R⁴ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁵ and R⁶taken together with the nitrogen to which they are attached form a 5- or 6- or 7-membered saturated ring optionally containing one other heteroatom selected from the group consisting of oxygen, nitrogen and sulphur;

R⁷ and R⁸ are each independently selected from the group consisting of hydrogen, C₁₋C₁₀ alkyl, C₂₋C₁₀ alkenyl, C₂₋C₁₀ alkynyl, C₃-C₈ cycloalkyl, C₃-C₈ heterocycloalkyl, aryl and heteroaryl,

R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³, R⁴, R⁵, R⁶, R⁷, R⁸, R⁹ is unsubstituted or, where possible, is substituted with one or more substituents independently selected from the group consisting of C₁-C₆ alkyl, aryl, heteroaryl, halogen, —OR¹, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁₋C₄ alkyl” and “C₁₋C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃₋C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids amd pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate(mesylate), ethanesulfonate(esylate), 2-hydroxyethanesulfonate, benzenesulfonate(besylate), p-aminobenzenesulfonate, p-toluenesulfonate(tosylate), and napthalene-2-sulfonate.

Compounds of particular interest include those wherein:

R¹ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R² is —OR⁹;

R³ is H or C₁₋C₄alkyl;

R⁴ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³, R⁴, R⁹ is unsubstituted or, where possible, is substituted with one or more substituents independently selected from the group consisting of C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl and heteroaryl, wherein R⁵ and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl.

Of further interest are those compounds where:

R¹ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁C₁₀alkyl-heteroaryl;

R² is —OR⁹;

R³ is H or C₁₋C₄alkyl;

R⁴ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R⁹ is H or a cation;
where any carbon or heteroatom of R¹, R², R³, R⁴ is unsubstituted or, where possible, is substituted with one or more substituents independently selected from C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
   or a pharmaceutically acceptable salt thereof

Yet a further group of compounds of interest include those wherein:

R¹ is cyclohexyl, 3-isopropyloxyphenyl, 3-fluorophenyl, 2,3-dichlorophenyl, or 3,5-dichlorophenyl;

R² is OH;

R³ is H; and

R⁴ is cyclohexyl, cycloheptyl, 2-thienyl, or phenyl; or

the pharmaceutically acceptable salts thereof

Processes for preparing the compound of formula (I) are also within the ambit of this invention. To illustrate, a process for preparing a compound of formula (I)

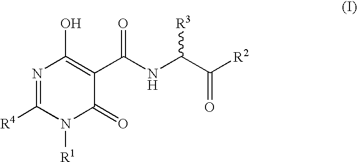

wherein R¹, R², R³ and R⁴ are the same as defined above for formula (I), the process comprising treating a compound of formula A:

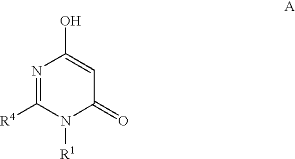

wherein R¹ and R⁴ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (I) where R² is —OEt;

or a process for preparing a compound of formula (I) wherein R¹, R², R³ and R⁴ are the same as defined above for formula (I), the process comprising treating a compound of formula B:

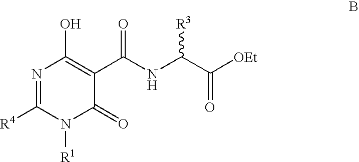

wherein R¹, R³ and R⁴ are the same as for those groups in formula (I) with an alkali such as sodium hydroxide, in an appropriate solvent, such as aqueous ethanol, at a suitable temperature such as room temperature, to form a compound of formula (I) where R² is —OH;

It will be appreciated by those skilled in the art that the compounds of formula (I) may exist in one or more tautomeric forms such as:

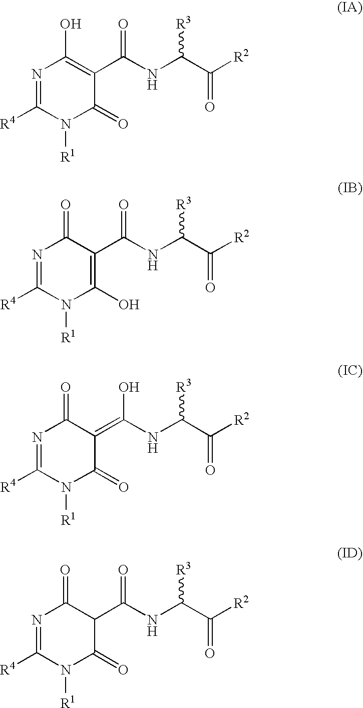

All tautomeric forms of the compounds described herein, including mixtures thereof, are intended to be encompassed within the scope of the invention. Generally, the compounds exemplified herein have been assigned names based on the structure of the tautomer of formaula (IA). It should be understood that any reference to named compounds of this invention is intended to encompass all tautomers of the named compounds and any mixtures of tautomers of the named compounds.

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

rt—room temperature
DBU—1,8-diazabicyclo[5.4.0]undec-7-ene
DCM—dichloromethane
DMF—dimethylformamide
DMSO—dimethylsulfoxide
RP-HPLC—reverse-phase high performance liquid chromatography
LCMS—liquid chromatography/mass spectrometry
NMR—nuclear magnetic resonance
ODS—octadecyl silyl
PTFE—polytetrafluoroethylene
TFA—Trifluoroacetic acid
THF—tetrahydrofuran

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

### Illustrated Methods of Preparation

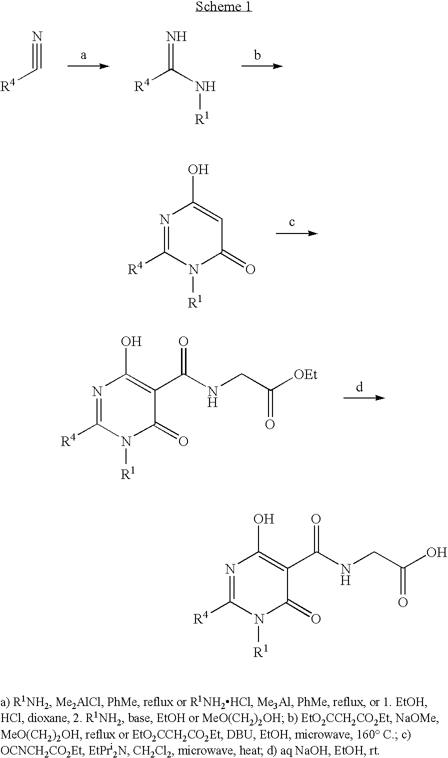

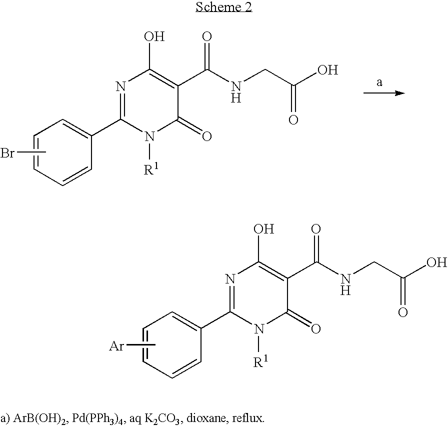

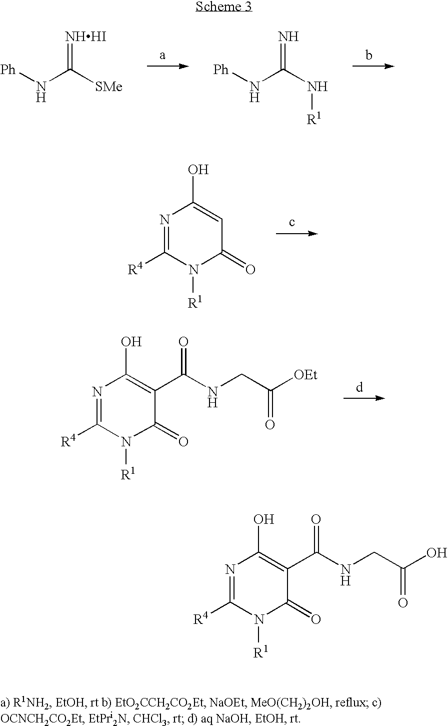

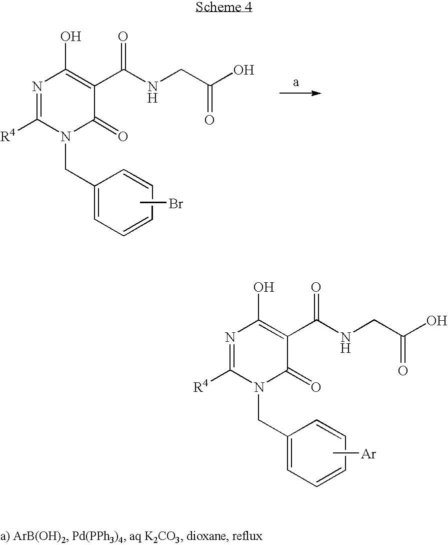

### Experimentals

### EXAMPLE 1

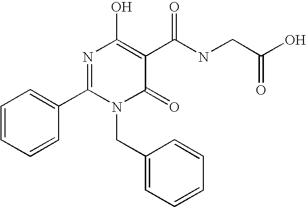

### N-{[4-Hydroxy-6-oxo-2-phenyl-1-(phenylmethyl)-1,6-dihydro-5-pyrimidinyl]carbonal}glycine

1a) N-(Phenylmethyl)benzenecarboximidamide. A mixture of copper (I) chloride (1.19 g, 12.0 mmoles), benzylamine (1.10 mL, 10.1 mmol) and benzonitrile (10 mL, 100 mmol) was stirred at 80° C. under nitrogen for 18 h, then cooled. 2M aqueous sodium hydroxide (50 mL) was added and the mixture extracted with ether. The extracts were washed with aqueous sodium hydroxide and brine, then dried (potassium carbonate, sodium sulphate). After filtering, 4M hydrogen chloride in dioxane (2.5 mL) was added and the solid filtered, washed with ether and dried to leave 1.40 g crude hydrochloride salt. 0.5 g of the salt was partitioned between 1M aqueous sodium hydroxide and ether. After washing and drying the extracts as before, the solvent was removed under reduced pressure to give the title compound (0.342 g, 45%). LCMS (ES⁻) m/z 211 (MH⁺).

1b) 6-Hydroxy-2-phenyl-3-(phenylmethyl)-4(3H)-pyrimidinone. A mixture of the compound from example 1(a) (0.342 g, 1.63 mmol), triethyl methanetricarboxylate (0.755 g, 3.25 mmol), methanolic sodium methoxide (0.378 mL of a 4.37 M solution, 1.65 mmol) and ethanol (3 mL) was stirred in a microwave reactor at 160° C. for 0.5 h, then cooled and poured into 1M aqueous hydrochloric acid (20 mL). The mixture was extracted with ethyl acetate, and the extracts washed with brine, dried (MgSO₄) and evaporated under reduced pressure. The residue was chromatographed (silica gel, 5-10% methanol/dichloromethane) to give the title compound (0.320 g, 44%) as a foam. 1H NMR (400 MHz, DMSO-d₆) δ ppm 5.05 (s, 2 H) 5.50 (s, 1 H) 6.87 (m, 2 H) 7.18-7.26 (m, 3 H) 7.36-7.43 (m,4H) 7.49 (m, 1 H) 11.64 (s, 1 H).

1c) Ethyl N-{[4-hydroxy-6-oxo-2-phenyl-1-(phenylmethyl)-1,6-dihydro-5-pyrimidinyl]carbonyl}glycinate. Ethyl 2-isocyanatoacetate (0.062 mL, 0.55 mmol) was injected into a stirred solution of the compound from example 1(b) (0.139 g, 0.500 mmol) and N,N-diisopropylethylamine (0. 174 mL, 1.00 mmol) in dichloromethane (3 mL) and the mixture stirred in a microwave reactor at 80° C. for 0.5 h. A further 0.062 mL of ethyl 2-isocyanatoacetate was added, and heating continued a further 0.5 h at 120° C. After cooling, the mixture was poured into 1M aqueous hydrochloric acid (10 mL) and extracted with dichloromethane. The extracts were dried (MgSO₄), evaporated under reduced pressure and the residue chromatographed (silica gel, 20-80% ethyl acetate/hexane) to give the title compound (0.105 g, 51%) as a white solid. 1H NMR (400 MHz, DMSO-d₆) δ ppm 1.21 (t, J=7.07 Hz, 3 H) 4.14 (q, J=7.07 Hz, 2 H) 4.17 (d, J=5.81 Hz, 2 H) 5.12 (s, 2 H) 7.00 (d, J=6.57 Hz, 2 H) 7.20-7.29 (m, 3 H) 7.44-7.55 (m, 5 H), 9.87 (t, J=5.81 Hz, 1 H) 15.89 (s, 1 H).

1d) N-{[4-Hydroxy-6-oxo-2-phenyl-1-(phenylmethyl)-1,6-dihydro-5-pyrimidinyl]carbonyl}glycine. 1 M aqueous sodium hydroxide (2.5 mL, 2.50 mmol) was added dropwise at room temperature to a stirred suspension of the compound from example 1(c) (0.103 g, 0.253 mmol) in methanol (15 mL). The mixture was stirred 3 h, diluted with water (30 mL) and acidified by adding 1 M aqueous hydrochloric acid (3 mL). The precipitate was filtered, washed with water and dried to give the title compound (0.039 g, 41%) as a white powder. 1H NMR (400 MHz, DMSO-d₆) δ ppm 4.10 (d, J=5.56 Hz, 2 H) 5.12 (s, 2 H) 7.00 (d, J=6.57 Hz, 2 H) 7.20-7.29 (m, 3 H) 7.44-7.51 (m, 4 H) 7.54 (m, 1 H) 9.84 (t, J=5.68 Hz, 1 H) 12.90 (s, 1 H) 16.02 (s, 1 H).

### EXAMPLE 2

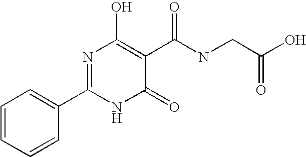

### N-[(4-Hydroxy-6-oxo-2-phenyl-1,6-dihydro-5-pyrimidinyl)carbonyl]glycine

2a) 6-Hydroxy-2-phenyl-4(1H)-pyrimidinone. Sodium hydride (0.800 g of a 60% oil suspension, 20.0 mmol) was added portionwise with cooling to a stirred mixture of benzamidine hydrochloride (1.56 g, 10.0 mmol), diethyl malonate (1.60 g, 10.0 mmol) and 2-methoxyethanol (14 mL). After the addition, the mixture was stirred at reflux under nitrogen for 18 h, then cooled and diluted with water (80 mL) and acidified with 1M aqueous hydrochloric acid (30 mL). The precipitate was filtered, washed with water and ether, then dried to leave the title compound (1.11 g, 59%) as a pale yellow solid. 1H NMR (400 MHz, DMSO-d₆) δ ppm 5.35 (s, 1 H) 7.50-7.61 (m, 3 H) 8.08-8.10 (m, 2 H), 11.80 (br s, 2 H).

2b) Ethyl N-[(4-hydroxy-6-oxo-2-phenyl-1,6-dihydro-5-pyrimidinyl)carbonyl]glycinate. A mixture of the compound from example 2(a) (0.200 g, 1.06 mmol), ethyl 2-isocyanatoacetate (0.170 mL, 1.52 mmol), N,N-diisopropylethylamine (0.350 mL, 2.01 mmol) and tetrahydrofuran (2 mL) was stirred in a microwave reactor at 130° C. for 0.5 h. A further 0.120 mL of ethyl 2-isocyanatoacetate was added, and heating continued a further 0.5 h at 150° C. A further 0.120 mL of ethyl 2-isocyanatoacetate and 0.350 mL of N,N-diisopropylethylamine were added, and heating continued a further 0.5 h at 180° C. After cooling, the mixture was poured into 1M aqueous hydrochloric acid (10 mL) and extracted with ethyl acetate. The extracts were washed with water and brine, dried (MgSO₄), evaporated under reduced pressure and the residue chromatographed (silica gel, 1-6% methanol/dichloromethane). The partially purified product was recrystallised (EtOH) to give the title compound (0.183 g, 54%) as a white solid. 1H NMR (400 MHz, DMSO-d₆) δ ppm 1.23 (t, J=7.07 Hz, 3 H) 4.16 (q, J=7.07 Hz, 2 H) 4.19 (d, J=5.81 Hz, 2 H) 7.58-7.60 (m, 2 H) 7.67 (m, 1 H) 8.15-8.19 (m, 2 H) 9.90 (t, J=5.69 Hz, 1H) 13.14 (s, 1 H)

2c) N-[(4-Hydroxy-6-oxo-2-phenyl-1,6-dihydro-5-pyrimidinyl)carbonyl]glycine. Aqueous sodium hydroxide (2.00 mL of a 1 M solution, 2.00 mmol) was added dropwise to a stirred suspension of the compound from example 2(b) (0.070 g, 0.221 mmol) in methanol (5 mL). The mixture was stirred for 18 h, then water (20 mL) added, followed by 1 M aqueous hydrochloric acid to pH 2. The precipitate was filtered, washed with water and dried to give the title compound (0.056 g, 88%) as a white powder. 1H NMR (400 MHz, DMSO-d₆) δ ppm 4.11 (d, J=5.81 Hz, 2 H) 7.55-7.61 (m, 2 H) 7.67 (m, 1 H) 8.14-8.20 (m, 2 H) 9.86 (t, J=5.68 Hz, 1 H) 13.00 (s, 1 H) 15.87 (s, 1 H).

### EXAMPLE 3

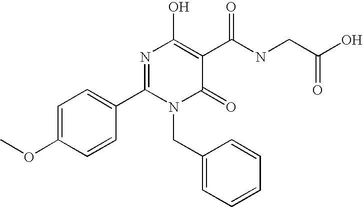

### N-{[4-Hydroxy-2-[4-(methyloxy)phenyl]-6-oxo-1-(phenylmethyl)-1,6-dihydro-5-pyrimidinyl]carbonyl glycine

3a) 4-(Methyloxy)-N-(phenylmethyl)benzenecarboximidamide hydrochloride. A 2 M solution of trimethylaluminium in toluene (2.50 mL, 5.00 mmol) was added dropwise to a stirred slurry of benzylamine hydrochloride (0.718 g, 5.00 mmol) in toluene (10 mL) under nitrogen. After stirring at ambient temperature for 0.5 h, 4-methoxybenzonitrile (0.665 g, 5.00 mmol) was added and the mixture refluxed for 4 h. After cooling in ice, 1 M aqueous sodium hydroxide (40 mL) was added and the mixture extracted with ether. The extracts were washed with brine, dried (K₂CO₃, Na₂SO₄) and filtered. 4 M hydrogen chloride in dioxane (2.5 mL) was added and the precipitate filtered, washed with ether and dried to give the title compound (1.25 g, 90%) as a white powder. 1H NMR (400 MHz, DMSO-d₆) δ ppm 3.87 (s, 3 H) 4.69 (d, J=6.06 Hz, 2 H) 7.16 (m, 2 H) 7.33-7.52 (m, 5 H) 7.81 (m, 2 H) 9.12 (s, 1 H) 9.46 (s, 1 H) 10.17 (t, J=5.56 Hz, 1 H).

3b) 6-Hydroxy-2-[4-(methyloxy)phenyl]-3-(phenylmethyl)-4(3H)-pyrimidinone. A mixture of the compound of example 3(a) (0.100 g, 0.362 mmol), diethyl malonate (0.116 mL, 0.724 mmol), sodium methoxide in methanol (0.166 mL of a 4.37 M solution, 0.724 mmol) and ethanol (1 mL) was microwaved at 160° C. for 0.5 h and at 180° C. for 0.5 h, then cooled and diluted with water (20 mL). 1 M aqueous hydrochloric acid (5 mL) was added and the mixture extracted with ethyl acetate. The extracts were dried (MgSO₄) and the solvent removed under reduced pressure to give the title compound (0.094 g, 50%) as a yellow powder. LCMS (ES⁺) m/z 309 (MH⁺).

3c) Ethyl N-{[4-hydroxy-2-[4-(methyloxy)phenyl]-6-oxo-1-(phenylmethyl)-1,6-dihydro-5-pyrimidinyl]carbonyl}glycinate. A mixture of the compound from example 3(b) (0.092 g, 0.298 mmol), ethyl 2-isocyanatoacetate (0.067 mL, 0.597 mmol), N,N-diisopropylethylamine (0.104 mL, 0.597 mmol) and tetrahydrofuran (2 mL) was stirred in a microwave reactor at 160° C. for 0.5 h. A further 0.067 mL of ethyl 2-isocyanatoacetate and 0.104 mL of N,N-diisopropylethylamine were added, and heating continued a further 1 h at 180° C. After cooling, the mixture was poured into 1M aqueous hydrochloric acid (10 mL) and extracted with ethyl acetate. The extracts were dried (MgSO₄), evaporated under reduced pressure and the residue chromatographed (silica gel, 20-80% ethyl acetate/hexane) to give the title compound (0.053 g, 41%) as a white solid. 1H NMR (400 MHz, CHLOROFORM-d) δ ppm 1.32 (t, J=7.20 Hz, 3 H) 3.86 (s, 3 H) 4.20 (d, J=5.81 Hz, 2 H) 4.26 (q, J=7.07 Hz, 2 H) 5.29 (s, 2 H) 6.91 (m, 2 H) 7.01-7.04 (m, 2 H) 7.26-7.35 (m, 3 H) 7.40 (m, 2 H) 9.98 (t, J=5.31 Hz, 1 H) 15.64 (s, 1 H).

3d) N-{[4-Hydroxy-2-[4-(methyloxy)phenyl]-6-oxo-1-(phenylmethyl)-1,6-dihydro-5-pyrimidinyl]carbonyl}glycine. Aqueous sodium hydroxide (1.00 mL of a 1 M solution, 1.00 mmol) was added dropwise to a stirred solution of the compound from example 3(c) (0.051 g, 0.117 mmol) in methanol (5 mL). The mixture was stirred for 2 h, then filtered and diluted with water (20 mL). 1 M aqueous hydrochloric acid was added to pH 2 and the mixture extracted with ethyl acetate. The extracts were dried (MgSO₄), evaporated under reduced pressure and the residue purified by reverse phase-HPLC (10-90% acetonitrile-water-0.1% TFA) to give the title compound (0.029 g, 60%) as a solid. 1H NMR (400 MHz, DMSO-d₆) δ ppm 3.80 (s, 3 H) 4.09 (d, J=5.56 Hz, 2 H) 5.18 (s, 2 H) 7.01 (d, J=8.84 Hz, 2 H) 7.03-7.06 (m, 2 H) 7.21-7.32 (m, 3 H) 7.48 (d, J=8.59 Hz, 2 H) 9.82 (t, J=5.56 Hz, 1 H) 12.89 (br s, 1 H) 15.94 (s, 1 H).

### EXAMPLE 4

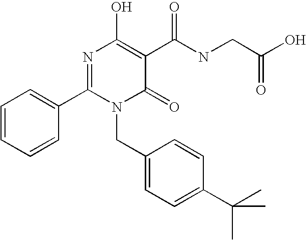

### N-[(1-{[4-(1,1-Dimethylethyl)phenyl]methyl}-4-hydroxy-6-oxo-2-phenyl-1,6-dihydro-5-pyrimidinyl)carbonyl]glycine

4a) N-{[4-(1,1-Dimethylethyl)phenyl]methyl}benzenecarboximidamide. A 1 M solution of dimethylaluminium chloride in hexane (5.00 mL, 5.00 mmol) was added dropwise to a stirred solution of 4-tertbutylbenzylamine (0.816 g, 5.00 mmol) in toluene (20 mL) under nitrogen. After stirring at ambient temperature for 0.5 h, benzonitrile (1.02 mL, 10.0 mmol) was added and the mixture refluxed for 6 h under nitrogen. After cooling in ice, 1 M aqueous sodium hydroxide (40 mL) was added and the mixture extracted with ether. The extracts were dried (K₂CO₃, Na₂SO₄), filtered, and 4 M hydrogen chloride in dioxane (2.5 mL) added. The mixture was washed with 1M aqueous hydrochloric acid and the aqueous phase adjusted to pH 14 with aqueous sodium hydroxide, then extracted with ether as above. After removal of the volatiles under reduced pressure, the crude amidine was obtained as a clear gum (1.00 g). LCMS (ES⁺) m/z 267 (MH⁺).

4b) 3-{[4-(1,1-Dimethylethyl)phenyl]methyl}-6-hydroxy-2-2-phenyl-4(3H)-pyrimidinone. A mixture of the compound of example 4(a) (0.300 g, 1.13 mmol), diethyl malonate (0.416 g, 2.60 mmol), sodium methoxide in methanol (0.500 mL of a 4.37 M solution, 2.18 mmol) and ethanol (2 mL) was microwaved at 160° C. for 1 h and at 180° C. for 0.5 h, then cooled and diluted with water (20 mL). 1 M aqueous hydrochloric acid (5 mL) was added and the mixture extracted with ethyl acetate. The extracts were washed with water and brine, then dried (MgSO₄) and the solvent removed under reduced pressure. The residue was chromatographed (silica gel, 2-8% methanol/dichloromethane) to give the title compound (0.171 g, 34% over 2 steps) as a solid. 1H NMR (400 MHz, DMSO-d₆) δ ppm 1.23 (s, 9 H) 5.01 (s, 2 H) 5.47 (s, 1 H) 6.82 (d, J=8.34 Hz, 2 H) 7.25 (d, J=8.34 Hz, 2 H) 7.42-7.53 (m, 5 H) 11.61 (s, 1 H).

4c) Ethyl N-[(1-{[4-(1,1-dimethylethyl)phenyl]methyl}-4-hydroxy-6-oxo-2-phenyl-1,6-dihydro-5-pyrimidinyl)carbonyl]glycinate. A mixture of the compound from example 4(b) (0.169 g, 0.505 mmol), ethyl 2-isocyanatoacetate (0.113 mL, 1.01 mmol), N,N-diisopropylethylamine (0.192 mL, 1.10 mmol) and dichloromethane (2 mL) was stirred in a microwave reactor at 120° C. for 1 h. After cooling, 1M aqueous hydrochloric acid (2 mL) was added and the mixture extracted with dichloromethane. The extracts were dried (MgSO₄), evaporated under reduced pressure and the residue chromatographed (silica gel, 1-6% methanol dichloromethane) to give the title compound (0.182 g, 78%) as a colourless gum. 1H NMR (400 MHz, DMSO-d₆) δ ppm 1.21 (t, J=7.20 Hz, 3 H) 1.23 (s, 9 H) 4.14 (q, J=7.07 Hz, 2 H) 4.16 (d, J=5.81 Hz, 2 H) 5.09 (s, 2 H) 6.95 (d, J=8.34 Hz, 2 H) 7.28 (d, J=8.34 Hz, 2 H) 7.47-7.58 (m, 5 H) 9.85 (t, J=5.68 Hz, 1 H) 15.86 (s, 1 H).

4d) N-[(1-{[4-(1,1-Dimethylethyl)phenyl]methyl-4-hydroxy-6-oxo-2-phenyl-1,6-dihydro-5-pyrimidinyl)carbonyl]glycine. Aqueous sodium hydroxide (2.00 mL of a 1 M solution, 2.00 mmol) was added dropwise to a stirred solution of the compound from example 4(c) (0.180 g, 0.388 mmol) in methanol (10 mL). The mixture was stirred for 2 h, then concentrated under reduced pressure and diluted with water (20 mL). 1 M aqueous hydrochloric acid was added to pH 2 and the precipitate filtered. The solid obtained contained 13% of the starting material by LCMS. It was redissolved in ethanol (10 mL) and aqueous sodium hydroxide (1.00 mL of a 1 M solution, 1.00 mmol) added dropwise. After 4 h, added water (50 mL) and acidified as before. The precipitate was filtered, washed with water and dried to give the title compound (0.154 g, 91%) as a solid. 1H NMR (400 MHz, DMSO-d₆) δ ppm 1.23 (s, 9 H) 4.08 (d, J=5.81 Hz, 2 H) 5.19 (s, 2 H) 6.95 (d, J=8.34 Hz, 2 H) 7.28 (d, J=8.34 Hz, 2 H) 7.47-7.58 (m, 5 H) 9.82 (t, J=5.56 Hz, 1 H) 12.90 (s, 1 H) 16.00 (s, 1 H).

### EXAMPLE 5

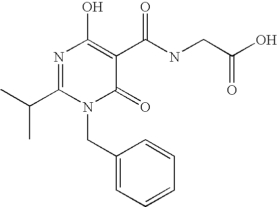

### N-{[4-Hydroxy-2-(1-methylethyl)-6-oxo-1-(phenylmethyl)-1,6-dihydro-5-pyrimidinyl]carbonyl}glycine

5a) 2-Methyl-N-(phenylmethyl)propanimidamide. Ethanol (1.16 mL, 20.0 mmol) was added to a solution of 2-methylpropanenitrile (0.691 g, 10.0 mmol) in 4M hydrogen chloride in dioxane (10 mL, 40 mmol). After 70 h, the solvent was removed under reduced pressure and tetrahydrofuran (20 mL) was added. Benzylamine (0.930 mL, 8.51 mmol) was injected with stirring, followed by triethylamine (2.00 mL, 14.3 mmol). The mixture was stirred for 4 h, then poured into 1 M aqueous hydrochloric acid (200 mL). The solution was washed with ether, then made alkaline with 6M aqueous sodium hydroxide and extracted with ether. The extracts were dried (K₂CO₃, Na₂SO₄) and evaporated under reduced pressure to give the title compound (1.23 g, 82%) as a clear oil. LCMS (ES⁺) m/z 177 (MH⁺).

5b) 6-Hydroxy-2-(1-methylethyl)-3-(phenylmethyl)-4(3H)-pyrimidinone. A mixture of the compound of example 5(a) (0.352 g, 2.00 mmol), diethyl malonate (0.640 g, 4.00 mmol), sodium methoxide in methanol (0.920 mL of a 4.37 M solution, 4.00 mmol) and ethanol (1 mL) was microwaved at 160° C. for 1 h, then cooled and diluted with water (25 mL). 6 M aqueous hydrochloric acid (1 mL) was added and the mixture extracted with ethyl acetate. The extracts were dried (MgSO₄), evaporated under reduced pressure and the residue was chromatographed (silica gel, 1-8% methanol/dichloromethane) to give the title compound (0.171 g, 35%) as a gummy solid. 1H NMR (400 MHz, DMSO-d₆) δ ppm 1.04 (d, J=6.57 Hz, 6 H) 3.04 (sept, J=6.65 Hz, 1 H) 5.30 (s, 2 H) 5.36 (s, 1 H) 7.13 (m, 2 H) 7.27 (t, J=7.33 Hz, 1 H) 7.35 (m, 2 H) 11.30 (s, 1 H).

5c) Ethyl N-{[4-hydroxy-2-(1-methylethyl)-6-oxo-1-(phenylmethyl)-1,6-dihydro-5-pyrimidinyl]carbonyl}glycinate. A mixture of the compound from example 5(b) (0.169 g, 0.692 mmol), ethyl 2-isocyanatoacetate (0.155 mL, 1.38 mmol), N,N-diisopropylethylamine (0.265 mL, 1.52 mmol) and dichloromethane (3 mL) was stirred in a microwave reactor at 120° C. for 1 h. After cooling, 1M aqueous hydrochloric acid (2 mL) was added and the mixture extracted with dichloromethane. The extracts were dried (MgSO₄), evaporated under reduced pressure and the residue chromatographed (silica gel, 0-5% methanol dichloromethane) to give the title compound (0.263 g) as a colourless gum. LCMS (ES⁺) m/z 374 (MH⁺).

5d) N-{[4-Hydroxy-2-(1-methylethyl)-6-oxo-1-(phenylmethyl)-1,6-dihydro-5-pyrimidinyl]carbonyl}glycine. Aqueous sodium hydroxide (5.00 mL of a 1 M solution, 5.00 mmol) was added dropwise to a stirred solution of the compound from example 5(c) (0.260 g, 0.696 mmol) in ethanol (20 mL). The mixture was stirred for 4 h, filtered, then 1 M aqueous hydrochloric acid was added to pH 2. The mixture was concentrated under reduced pressure to about 15 mL, water (50 mL) added and the mixture extracted with ethyl acetate. The extracts were washed with water, dried (MgSO₄), evaporated under reduced pressure. The residue was dissolved in methanol and water added with warming until cloudy. After cooling, the precipitate was filtered, washed with water and dried to give the title compound (0.062 g) as a solid. A second crop was obtained from the mother liquor (0.067 g, 54% overall). 1H NMR (400 MHz, DMSO-d₆) δ ppm 1.08 (d, J=6.57 Hz, 6 H) 3.13 (sept, J=6.57 Hz, 1 H) 4.08 (d, J=5.56 Hz, 2 H) 5.38 (s, 2 H) 7.19 (m, 2 H) 7.29 (m, 1 H) 7.37 (m, 2 H) 9.84 (t, J=5.56 Hz, 1 H) 12.90 (s, 1 H) 15.86 (s, 1 H).

### EXAMPLE 6

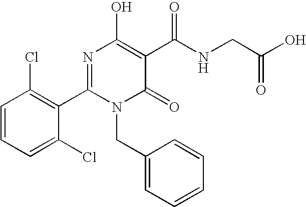

### N-{[2-(2,6-Dichlorophenyl)-4-hydroxy-6-oxo-1-(phenylmethyl)-1,6-dihydro-5-pyrimidinyl]carbonyl}glycine

6a) 2-(2,6-Dichlorophenyl)-6-hydroxy-3-(phenylmethyl)-4(3H)-pyrimidinone. A 1 M solution of dimethylaluminium chloride in hexane (5.50 mL, 5.50 mmol) was added dropwise to a stirred solution of benzylamine (0.536 g, 5.00 mmol) in toluene (20 mL) under nitrogen. After stirring at ambient temperature for 20 min, 2,6-dichlorobenzonitrile (1.72 g, 10.0 mmol) was added and the mixture refluxed for 18 h under nitrogen. After cooling, 1 M aqueous sodium hydroxide (40 mL) was added and the mixture extracted with ether. The extracts were dried (K₂CO₃, Na₂SO₄) and evaporated under reduced pressure to leave a solid (2.33 g). A mixture of this solid (0.837 g), diethyl malonate (0.960 g, 6.00 mmol), sodium methoxide in methanol (0.686 mL of a 4.37 M solution, 3.00 mmol) and 2-methoxyethanol (10 mL) was refluxed under nitrogen for 18 h, then cooled and diluted with water (35 mL). 6 M aqueous hydrochloric acid (2 mL) was added, the mixture stirred 0.5 h, and the precipitate filtered, washed with water, then ether and dried to give the title compound (0.477 g, 76%) as a cream solid. 1H NMR (400 MHz, DMSO-d₆) δ ppm 4.91 (s, 2 H) 5.62 (s, 1 H) 6.80-6.86 (m, 2 H) 7.14-7.25 (m, 3 H) 7.56-7.61 (m, 3 H) 11.92 (br. s, 1 H).

6b) N-{[2-(2,6-Dichlorophenyl)-4-hydroxy-6-oxo-1-(phenylmethyl)-1,6-dihydro-5-pyrimidinyl]carbonyl}glycine. A mixture of 2-(2,6-dichlorophenyl)-6-hydroxy-3-(phenylmethyl)-4(3H)-pyrimidinone (0.174 g, 0.500 mmol), ethyl 2-isocyanatoacetate (0.112 mL, 1.00 mmol), N,N-diisopropylethylamine (0.192 mL, 1.10 mmol) and dichloromethane (2 mL) was stirred in a microwave reactor at 120° C. for 1 h. After cooling, the mixture was evaporated under reduced pressure and the residue dissolved in ethanol (15 mL). 1 M aqueous sodium hydroxide (3.00 mL, 3.00 mmol) was added dropwise and the mixture stirred for 1 h, then filtered and diluted with water (20 mL). 6 M aqueous hydrochloric acid was added to pH 2 and the mixture extracted with ethyl acetate. The extracts were dried (MgSO₄) and evaporated under reduced pressure. The residue was redissolved in methanol (10 mL) and 1 M aqueous sodium hydroxide (10.0 mL, 10.0 mmol) and the solution acidified as before. The precipitate was filtered, washed with water and dried to give the title compound (0.191 g, 85%) as a cream solid. 1H NMR (400 MHz, DMSO-d₆) δ ppm 4.14 (d, J=5.56 Hz, 2 H) 5.03 (s, 2 H) 6.87-6.92 (m, 2 H) 7.15-7.30 (m, 3 H) 7.58-7.68 (m, 3 H) 9.89 (t, J=5.56 Hz, 1 H) 13.00 (br. s., 1 H).

### EXAMPLE 7

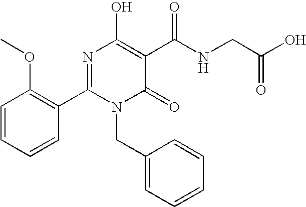

### N-{[4-Hydroxy-2-[2-(methyloxy)phenyl]-6-oxo-1-(phenylmethyl)-1,6-dihydro-5-pyrimidinyl]carbonyl}glycine

7a) 6-Hydroxy-2-[2-(methyloxy)phenyl]-3-(phenylmethyl)-4(3H)-pyrimidinone. A 1 M solution of dimethylaluminium chloride in hexane (5.50 mL, 5.50 mmol) was added dropwise to a stirred solution of benzylamine (0.536 g, 5.00 mmol) in toluene (20 mL) under nitrogen. After stirring at ambient temperature for 20 min, 2-methoxybenzonitrile (1.33 g, 10.0 mmol) was added and the mixture refluxed for 18 h under nitrogen. After cooling, 1 M aqueous sodium hydroxide (40 mL) was added and the mixture concentrated under reduced pressure to remove the organic solvents, then extracted with ether. The extracts were dried (K₂CO₃, Na₂SO₄) and evaporated under reduced pressure to leave the crude amidine (1.12 g). A mixture of the crude amidine (0.268 g), diethyl malonate (0.359 g, 2.24 mmol), sodium methoxide in methanol (0.513 mL of a 4.37 M solution, 2.24 mmol) and 2-methoxyethanol (5 mL) was refluxed under nitrogen for 18 h, then cooled and diluted with water (20 mL). 6 M aqueous hydrochloric acid (2 mL) was added, the mixture stirred 0.5 h, and the precipitate filtered, washed with water and dried to give the title compound (0.283 g, 76%) as a solid. 1H NMR (400 MHz, DMSO-d₆) δ ppm 3.64 (s, 3 H) 4.69 (d, J=15.41 Hz, 1 H) 5.13 (d, J=15.66 Hz, 1 H) 5.47 (s, 1 H) 6.79-6.87 (m, 2 H) 6.95 (t, J=7.45 Hz, 1 H) 7.11 (d, J=8.34 Hz, 1 H) 7.14-7.23 (m, 4 H) 7.42-7.50 (m, 1 H) 11.63 (br. s., 1 H).

7b) N-{[4-Hydroxy-2-[2-(methyloxy)phenyl]-6-oxo-1-(phenylmethyl)-1,6-dihydro-5-pyrimidinyl]carbonyl}glycine. A mixture of 6-hydroxy-2-[2-(methyloxy)phenyl]-3-(phenylmethyl)-4(3H)-pyrimidinone (0.154 g, 0.500 mmol), ethyl 2-isocyanatoacetate (0.112 mL, 1.00 mmol), N,N-diisopropylethylamine (0.192 mL, 1.10 mmol) and dichloromethane (2 mL) was stirred in a microwave reactor at 120° C. for 1 h. After cooling, the mixture was evaporated under reduced pressure and the residue dissolved in ethanol (15 mL). 1 M aqueous sodium hydroxide (3.00 mL, 3.00 mmol) was added dropwise and the mixture stirred for 1 h, then filtered and diluted with water (20 mL). 6 M aqueous hydrochloric acid was added to pH 2 and the mixture extracted with ethyl acetate. The extracts were dried (MgSO₄) and evaporated under reduced pressure and the residue purified by HPLC (ODS, 10-90% acetonitrile/water+0.1% trifluoroacetic acid). The product was redissolved in methanol (10 mL) and 1 M aqueous sodium hydroxide (10.0 mL, 10.0 mmol) and the solution acidified as before. The precipitate was filtered, washed with water and dried to give the title compound (0.053 g, 26%) as an orange solid. 1H NMR (400 MHz, DMSO-d₆) δ ppm 3.65 (s, 3 H) 4.10 (d, J=5.81 Hz, 2 H) 4.86 (d, J=15.66 Hz, 1 H) 5.14 (d, J=15.41 Hz, 1 H) 6.89-6.95 (m, 2 H) 7.02 (t, J=7.45 Hz, 1 H) 7.13 (d, J=8.34 Hz, 1 H) 7.18-7.26 (m, 3 H) 7.29-7.37 (m, 1 H) 7.49-7.55 (m, 1 H) 9.84 (t, J=5.56 Hz, 1 H) 12.91 (br. s., 1 H) 16.03 (s, 1 H).

### EXAMPLE 8

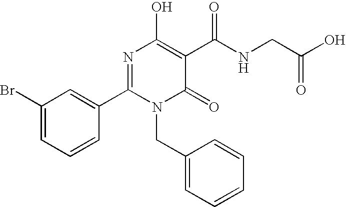

### N-{[2-(3-Bromophenyl)-4-hydroxy-6-oxo-1-(phenylmethyl)-1,6-dihydro-5-pyrimidinyl]carbonyl}glycine

8a) 2-(3-Bromophenyl)-6-hydroxy-3-(phenylmethyl)-4(3H)-pyrimidinone. A 1 M solution of dimethylaluminium chloride in hexane (5.50 mL, 5.50 mmol) was added dropwise to a stirred solution of benzylamine (0.536 g, 5.00 mmol) in toluene (20 mL) under nitrogen. After stirring at ambient temperature for 20 min, 3-bromobenzonitrile (1.82 g, 10.0 mmol) was added and the mixture refluxed for 18 h under nitrogen. After cooling, 1 M aqueous sodium hydroxide (40 mL) was added and the mixture extracted with ether. The extracts were washed with 1M aqueous hydrochloric acid. The aqueous phase was washed with ether, then adjusted to pH 13-14 with 6M aqueous sodium hydroxide and extracted again with ether. The extracts were dried (K₂CO₃, Na₂SO₄) and evaporated under reduced pressure to leave the crude amidine. A mixture of the crude amidine, diethyl malonate (1.60 g, 10.0 mmol), sodium methoxide in methanol (1.14 mL of a 4.37 M solution, 5.00 mmol) and 2-methoxyethanol (15 mL) was refluxed under nitrogen for 18 h, then cooled and diluted with water (30 mL). 6 M aqueous hydrochloric acid was added to adjust to pH 1-2, more water (50 mL) added and the mixture stirred 0.5 h. The precipitate was filtered, washed with water and ether, and dried to give the title compound (0.895 g, 50%) as a pale yellow powder. 1H NMR (400 MHz, DMSO-d₆) δ ppm 5.02 (s, 2 H) 5.52 (s, 1 H) 6.82-6.94 (m, 2 H) 7.18-7.29 (m, 3 H) 7.31-7.41 (m, 2 H) 7.50-7.56 (m, 1 H) 7.66-7.72 (m, 1 H) 11.68 (br. s., 1 H).

8b) Ethyl N-{[2-(3-bromophenyl)-4-hydroxy-6-oxo-1-(phenylmethyl)-1,6-dihydro-5-pyrimidinyl]carbonyl}glycinate. A mixture of 2-(3-bromophenyl)-6-hydroxy-3-(phenylmethyl)-4(3H)-pyrimidinone (0.357 g, 1.00 mmol), ethyl 2-isocyanatoacetate (0.224 mL, 2.00 mmol), N,N-diisopropylethylamine (0.384 mL, 2.20 mmol) and dichloromethane (2 mL) was stirred in a microwave reactor at 120° C. for 1 h. After cooling, the mixture was poured into 1M aqueous hydrochloric acid (4 mL) and extracted with dichloromethane. The extracts were dried (MgSO₄), evaporated under reduced pressure and chromatographed (silica gel, 1-8% methanol/dichloromethane) to give the title compound (0.245 g, 50%) as a colourless gum. LCMS (ES⁺) m/z 486/488 (MH)⁺.

8c) N-{[2-(3-Bromophenyl)-4-hydroxy-6-oxo-1-(phenylmethyl)-1,6-dihydro-5-pyrimidinyl]carbonyl}glycine (as the disodium salt). 1 M aqueous sodium hydroxide (5.00 mL, 5.00 mmol) was added dropwise to a stirred solution of ethyl N-{[2-(3-bromophenyl)-4-hydroxy-6-oxo-1-(phenylmethyl)-1,6-dihydro-5-pyrimidinyl]carbonyl}glycinate (0.243 g, 0.500 mmol) in ethanol (20 mL) and the mixture stirred at room temperature for 3 h. The precipitate was filtered, washed with ethanol and dried to give the title compound as the disodium salt (0.192 g, 76%) as a white solid. 1H NMR (400 MHz, DMSO-d₆) δ ppm 3.49 (d, J=4.29 Hz, 2 H) 4.93 (s, 2 H) 6.83-6.90 (m, 2 H) 7.13-7.36 (m, 6 H) 7.57-7.63 (m, 1 H) 10.17 (t, J=4.29 Hz, 1 H).

### EXAMPLE 9

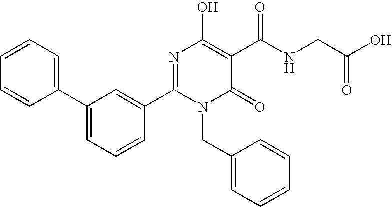

### N-{[2-(3-Biphenylyl)-4-hydroxy-6-oxo-1-(phenylmethyl)-1,6-dihydro-5-pyrimidinyl]carbonyl}glycine

A mixture of N-{[2-(3-bromophenyl)-4-hydroxy-6-oxo-1-(phenylmethyl)-1,6-dihydro-5-pyrimidinyl]carbonyl}glycine,disodium salt (0.122 g, 0.243 mmol), phenylboronic acid (0.044 g, 0.365 mmol), tetrakis(triphenylphosphine)palladium (0) (0.014 g, 0.012 mmol), 2M aqueous potassium carbonate (2.00 mL, 4.00 mmol) and dioxane (2 mL) was stirred at reflux under nitrogen for 6 h, then cooled. After acidifying to pH 1 with 1M aqueous hydrochloric acid, the mixture was extracted with ethyl acetate. The extracts were dried (MgSO₄), evaporated under reduced pressure and the residue purified by HPLC (ODS, 10-90% acetonitrile/water+0.1% trifluoroacetic acid). The product was additionally washed with warm ethanol and dried to give the title compound (0.042 g, 38%) as a solid. 1H NMR (400 MHz, DMSO-d₆) δ ppm 4.11 (d, J=5.56 Hz, 2 H) 5.16 (s, 2 H) 7.05-7.11 (m, 2 H) 7.23-7.33 (m, 3 H) 7.33-7.46 (m, 5 H) 7.49-7.54 (m, 1 H) 7.57 (t, J=7.71 Hz, 1 H) 7.67-7.71 (m, 1 H) 7.78-7.86 (m, 1 H) 9.87 (t, J=5.56 Hz, 1 H) 12.91 (br. s., 1 H) 16.05 (s, 1 H).

### EXAMPLE 10

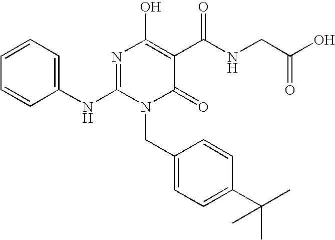

### N-{[1-{[4-(1,1-Dimethylethyl)phenyl]methyl}-4-hydroxy-6-oxo-2-(phenylamino)-1,6-dihydro-5-pyrimidinyl]carbonyl}glycine

10a) N-{[4-(1,1-Dimethylethyl)phenyl]methyl}-N′-phenylguanidine. A mixture of methyl N-phenylimidothiocarbamate hydroiodide (1.4 g, 4.75 mmol) and 4-t-butyl benzylamine (1.67 g, 9.5 mmol) was stirred in ethanol overnight. Nitrogen was then bubbled through the mixture for 30 minutes and then evaporated. Trituration in hexanes gave a solid (2.2 g, contained some starting t-butylbenzylamine hydroiodide salt). 1H NMR (400 MHz, DMSO-d₆) δ ppm 7.34-7.47 (m, 5 H), 7.25-7.35 (m, 3 H), 7.12 (d, J=7.58 Hz, 1 H), 3.84 (s, 2 H), 1.28 (s, 9 H).

10b) 1-{[4-(1,1-Dimethylethyl)phenyl]methyl}-2-(phenylamino)-4,6(1H,5H)-pyrimidinedione. A mixture of N-{[4-(1,1-dimethylethyl)phenyl]methyl}-N-phenylguanidine (2.1 g, 7.46 mmol) and diethyl malonate (2.0 mL, 13.17 mmol) in methoxyethanol (20 mL) was treated with sodium ethoxide (21% solution in ethanol, 2.0 mL) under nitrogen and heated under reflux for 24 hours. The mixture was cooled, diluted with 1 molar hydrochloric acid and extracted with ethyl acetate (×2). The combined extracts were washed with 1 molar hydrochloric acid and evaporated onto silica gel. Flash chromatography (10% ethyl acetate in hexanes eluting to 40% ethyl acetate in hexanes) gave the title compound (750 mg, 28%). 1H NMR (400 MHz, DMSO-d₆) δ ppm 10.77 (s, 1 H), 8.75 (s, 1 H), 7.40-7.49 (m, 2 H), 7.36 (d, J=8.59 Hz, 2 H), 7.28-7.34 (m, 2 H), 7.17 (d, J=8.34 Hz, 2 H), 7.10 (dd, J=7.33 Hz, 1 H), 5.34 (s, 2 H), 5.00 (s, 1 H), 1.25 (s, 9 H).

10c) Ethyl N-{[1-{[4-(1,1-dimethylethyl)phenyl]methyl}-4-hydroxy-6-oxo-2-(phenylamino)-1,6-dihydro-5-pyrimidinyl]carbonyl}glycinate. Ethyl isocyanatoacetate (240 uL, 2.14 mmol) was added to a solution of 1-{[4-(1,1-dimethylethyl)phenyl]methyl}-2-(phenylamino)-4,6(1H,5H)-pyrimidinedione (750 mg, 2.14 mmol) and diisopropylethylamine (740 uL, 4.28 mmol) in chloroform (30 mL) and stirred for 6 hours. The mixture was washed with 1 molar hydrochloric acid and evaporated onto silica gel. Flash chromatography (30% ethyl acetate in hexanes) gave the title compound (750 mg, 73%). 1H NMR (400 MHz, DMSO-d₆) δ ppm 15.64 (s, 1 H), 9.66 (t, J=5.81 Hz, 1 H), 9.56 (s, 1 H), 7.34-7.44 (m, 6 H), 7.22 (d, J=8.59 Hz, 3 H), 5.37 (s, 2 H), 4.12 (q, 2 H), 4.09 (d, J=5.56 Hz, 2 H), 1.26 (s, 9 H), 1.20 (t, J=7.20 Hz, 3 H).

10d) N-{[1-{[4-(1,1-Dimethylethyl)phenyl]methyl}-4-hydroxy-6-oxo-2-(phenylamino)-1,6-dihydro-5-pyrimidinyl]carbonyl}glycine. Ethyl N-{[1-{[4-(1,1-dimethylethyl)phenyl]methyl}-4-hydroxy-6-oxo-2-(phenylamino)-1,6-dihydro-5-pyrimidinyl]carbonyl}glycinate (730 mg, 1.52 mmol)was stirred in a mixture of ethanol (15 mL), 1 molar sodium hydroxide solution (3.0 mL) and 6 molar sodium hydroxide solution (1.5 mL) for 4 hours, depositing a solid over this time. The mixture was diluted with 1 molar hydrochloric acid solution, which, in turn gave a solid that was collected, washed with molar hydrochloric acid solution, water and hexanes. The solid was dried in vacuo to give the title compound (300 mg, 44%). 1H NMR (400 MHz, DMSO-d₆) δ ppm 15.79 (s, 1 H), 12.82 (s, 1 H), 9.63 (t, J=5.56 Hz, 1 H), 9.54 (s, 1 H), 7.35-7.44 (m, 6 H), 7.16-7.29 (m, 3 H), 5.37 (s, 2 H), 4.02 (d, J=5.56 Hz, 2 H), 1.26 (s, 9 H).

### EXAMPLE 11

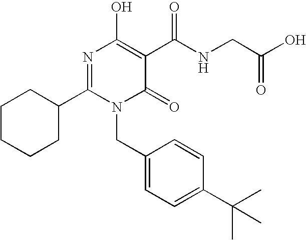

### N-[(2-Cyclohexyl-1-{[4-(1,1-dimethylethyl)phenyl]methyl}-4-hydroxy-6-oxo-1,6-dihydro-5-pyrimidinyl)carbonyl]glycine

11a) Ethyl cyclohexanecarboximidoate hydrochloride. A mixture of cyclohexanecarbonitrile (4.37 g, 40.0 mmol), ethanol (2.80 mL, 48.0 mmol) and 4M hydrogen chloride in dioxane (40 mL, 160 mmol) was allowed to stand at room temperature for 48 h, then the solvent removed under reduced pressure until a precipitate appeared. Ether (50 mL) was added and the solid filtered, washed with ether and dried at 50° C. under reduced pressure to give the title compound (5.66 g, 74%) as a white solid. 1H NMR (400 MHz, DMSO-d₆) δ ppm 1.09-1.31 (m, 3 H) 1.35 (t, J=6.95 Hz, 3 H) 1.37-1.49 (m, 2 H) 1.60-1.68 (m, 1 H) 1.71-1.79 (m, 2 H) 1.81-1.90 (m, 2 H) 2.66 (tt, J=11.81, 3.34 Hz, 1 H) 4.41 (q, J=6.99 Hz, 2 H) 11.31 (br. s., 2 H).

11b) 2-Cyclohexyl-3-{[4-(1,1-dimethylethyl)phenyl]methyl}-6-hydroxy-4(3H)-pyrimidinone. 4-tert-Butylbenzylamine (0.176 mL, 1.00 mmol) was added to a stirred solution of ethyl cyclohexanecarboximidoate hydrochloride (0.192 g, 1.00 mmol) in ethanol (1 mL), followed by 1,8-diazabicyclo[5.4.0]undec-7-ene (0.150 mL, 1.00 mmol) and the mixture stirred for 1 h. Diethyl malonate (0.320 g, 2.00 mmol) and more 1,8-diazabicyclo[5.4.0]undec-7-ene (0.150 mL, 1.00 mmol) were added and the mixture stirred in a microwave reactor at 160° C. for 1 h. After cooling, trifluoroacetic acid (0.162 mL, 2.10 mmol) was added and the mixture chromatographed (silica gel, 1-6% methanol/dichloromethane) to give the title compound (0.115 g, 34%) as a colourless gum. 1H NMR (400 MHz, CHLOROFORM-d) δ ppm 1.09-1.23 (m, 2 H) 1.24-1.39 (m, 1 H) 1.31 (s, 9 H) 1.47-1.58 (m, 2 H) 1.63-1.87 (m, 5 H) 2.84-2.97 (m, 1 H) 5.36 (s, 2 H) 5.61 (s, 1 H) 6.71 (br. s., 1 H) 7.09-7.17 (m, 2 H) 7.33-7.42 (m, 2 H).

11c) N-[(2-Cyclohexyl-1-{[4-(1,1-dimethylethyl)phenyl]methyl}-4-hydroxy-6-oxo-1,6-dihydro-5-pyrimidinyl)carbonyl]glycine. A mixture of 2-cyclohexyl-3-{[4-(1,1-dimethylethyl)phenyl]methyl}-6-hydroxy-4(3H)-pyrimidinone (0.115 g, 0.338 mmol), ethyl 2-isocyanatoacetate (0.076 mL, 0.677 mmol), N,N-diisopropylethylamine (0.129 mL, 0.741 mmol) and dichloromethane (1.5 mL) was stirred in a microwave reactor at 120° C. for 1 h. After cooling, acetic acid (0.045 mL, 0.786 mmol) was added and the mixture chromatographed (silica gel, 0-5% methanol/dichloromethane) to give the intermediate ester, sufficiently pure for use in the next step (LCMS). 1 M aqueous sodium hydroxide (2.00 mL, 2.00 mmol) was added dropwise to a stirred solution of the intermediate ester in ethanol (10 mL) at room temperature and the mixture stirred for 3 h, then concentrated to a volume of about 3 mL under reduced pressure and filtered. The filtrate was diluted with water (20 mL), acidified with 6 M aqueous hydrochloric acid to pH 2 and extracted with ethyl acetate. The extracts were washed with water and brine, dried (MgSO₄) and evaporated under reduced pressure. The residue was chromatographed (silica gel, 1-10% methanol/dichloromethane+0.5% acetic acid) to give the title compound (0.054 g, 36%) as a foam. 1H NMR (400 MHz, DMSO-d₆) δ ppm 1.10-1.29 (m, 3 H) 1.25 (s, 9 H) 1.39-1.72 (m, 7 H) 2.78-2.93 (m, 1 H) 4.06 (d, J=5.56 Hz, 2 H) 5.33 (s, 2 H) 7.13 (d, J=8.34 Hz, 2 H) 7.39 (d, J=8.34 Hz, 2 H) 9.83 (t, J=5.68 Hz, 1 H) 12.90 (br. s., 1 H) 15.82 (s, 1 H).

### EXAMPLE 12

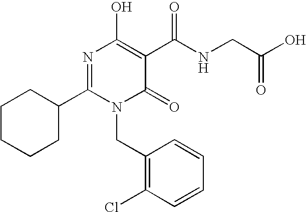

### N-({1-[(2-Chlorophenyl)methyl]-2-cyclohexyl-4-hydroxy-6-oxo-1,6-dihydro-5-pyrimidinyl}carbonyl)glycine

12a) 3-[(2-Chlorophenyl)methyl]-2-cyclohexyl-6-hydroxy-4(3H)-pyrimidinone. 2-Chlorobenzylamine (0.121 mL, 1.00 mmol) was added to a stirred solution of ethyl cyclohexanecarboximidoate hydrochloride (0.192 g, 1.00 mmol) and 1,8-diazabicyclo[5.4.0]undec-7-ene (0.150 mL, 1.00 mmol) in dioxane (2 mL) and the mixture stirred at 60° C. for 2 h under nitrogen, then cooled. Diethyl malonate (0.303 mL, 2.00 mmol) and more 1,8-diazabicyclo[5.4.0]undec-7-ene (0.150 mL, 1.00 mmol) were added and the mixture stirred in a microwave reactor at 160° C. for 1 h. After cooling, acetic acid (0.120 g, 2.00 mmol) was added and the mixture chromatographed (silica gel, 1-8% methanol/dichloromethane) to give the title compound (0.119 g, 37%) as a gum. LCMS (ES⁺) m/z 319 (MH⁺)

12b) N-({1-[(2-Chlorophenyl)methyl]-2-cyclohexyl-4-hydroxy-6-oxo-1,6-dihydro-5-pyrimidinyl}carbonyl)glycine. A mixture of 3-[(2-chlorophenyl)methyl]-2-cyclohexyl-6-hydroxy-4(3H)-pyrimidinone (0.117 g), ethyl 2-isocyanatoacetate (0.076 mL, 0.677 mmol), N,N-diisopropylethylamine (0.129 mL, 0.741 mmol) and dichloromethane (1.5 mL) was stirred in a microwave reactor at 120° C. for 1 h. After cooling, acetic acid (0.045 mL, 0.786 mmol) was added and the mixture chromatographed (silica gel, 0-5% methanol/dichloromethane) to give the intermediate ester, sufficiently pure for use in the next step (LCMS). 1 M aqueous sodium hydroxide (0.50 mL, 0.50 mmol) was added dropwise to a stirred solution of the intermediate ester in ethanol (5 mL) at room temperature and the mixture stirred for 2 h, then acidified with 6 M aqueous hydrochloric acid to pH 2. After concentrating under reduced pressure and diluting with water, the mixture was extracted with ethyl acetate. The extracts were dried (MgSO₄) and evaporated under reduced pressure. The residue was purified by HPLC (ODS, 10-90% acetonitrile/water+0.1% trifluoroacetic acid) to give the title compound (0.033 g, 21%) as a foam. 1H NMR (400 MHz, DMSO-d₆) δ ppm 1.10-1.23 (m, 3 H) 1.42-1.56 (m, 2 H) 1.57-1.73 (m, 5 H) 2.58-2.70 (m, 1 H) 4.06 (d, J=5.81 Hz, 2 H) 5.38 (s, 2 H) 6.93 (dd, J=7.45, 1.89 Hz, 1 H) 7.28-7.40 (m, 2 H) 7.55 (dd, J=7.58, 1.52 Hz, 1 H) 9.77 (t, J=5.56 Hz, 1 H) 12.86 (br. s., 1 H) 15.88 (s, 1 H).

### EXAMPLE 13

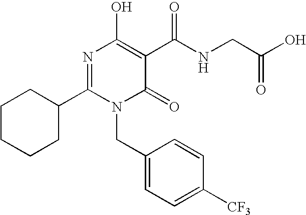

### N-[(2-Cyclohexyl-4-hydroxy-6-oxo-1-{[4-(trifluoromethyl)phenyl]methyl}-1,6-dihydro-5-pyrimidinyl)carbonyl]glycine

13a) 2-Cyclohexyl-6-hydroxy-3-{4-(trifluoromethyl)phenyl]methyl}-4(3H)-pyrimidinone. A mixture of 4-(trifluoromethyl)benzylamine (0.142 mL, 1.00 mmol), ethyl cyclohexanecarboximidoate hydrochloride (0.192 g, 1.00 mmol), N,N-diisopropylethylamine (0.174 mL, 1.00 mmol) and ethanol (1 mL) was stirred at room temperature for 18 h, then diethyl malonate (0.182 g, 1.20 mmol) and 1,8-diazabicyclo[5.4.0]undec-7-ene (0.300 mL, 2.01 mmol) were added and the mixture stirred in a microwave reactor at 160° C. for 1 h. After cooling, acetic acid (0.180 mL, 3.00 mmol) was added and the mixture chromatographed (silica gel, 1-6% methanol/dichloromethane) to give the title compound (0.115 g, 33%) as a gum. 1H NMR (400 MHz, DMSO-d₆) δ ppm 1.09-1.30 (m, 3 H) 1.38-1.75 (m, 7 H) 2.61-2.75 (m, 1 H) 5.36 (s, 1 H) 5.39 (s, 2 H) 7.39 (d, J=8.08 Hz, 2 H) 7.73 (d, J=8.08 Hz, 2 H) 11.55 (br. s., 1 H).

13b) N-[(2-Cyclohexyl-4-hydroxy-6-oxo-1-{[4-(trifluoromethyl)phenyl]methyl}-1,6-dihydro-5-pyrimidinyl)carbonyl]glycine. A mixture of 2-cyclohexyl-6-hydroxy-3-{[4-(trifluoromethyl)phenyl]methyl}-4(3H)-pyrimidinone (0.114 g, 0.324 mmol), ethyl 2-isocyanatoacetate (0.073 mL, 0.647 mmol), N,N-diisopropylethylamine (0.113 mL, 0.647 mmol) and dichloromethane (1.5 mL) was stirred in a microwave reactor at 120° C. for 1 h. After cooling, the solvent was removed under reduced pressure and the residue azeotroped with methanol twice, then suspended in ethanol (5 mL). 2 M aqueous sodium hydroxide (1.50 mL, 3.00 mmol) was added dropwise to the suspension at room temperature and the mixture stirred for 1 h, then diluted with water (50 mL), acidified with 6 M aqueous hydrochloric acid to pH 1-2 and extracted with ethyl acetate. The extracts were washed with water and brine, dried (MgSO₄) and evaporated under reduced pressure. The residue was purified by HPLC (ODS, 10-90% acetonitrile/water+0.1% trifluoroacetic acid) to give the title compound (0.057 g, 39%) as a foam. 1H NMR (400 MHz, DMSO-d₆) δ ppm 1.12-1.25 (m, 3 H) 1.40-1.73 (m, 7 H) 2.72-2.86 (m, 1 H) 4.07 (d, J=5.56 Hz, 2 H) 5.47 (s, 2 H) 7.46 (d, J=8.08 Hz, 2 H) 7.74 (d, J=8.34 Hz, 2 H) 9.79 (t, J=5.56 Hz, 1 H) 12.88 (br. s., 1 H) 15.86 (s, 1 H).

### EXAMPLE 14

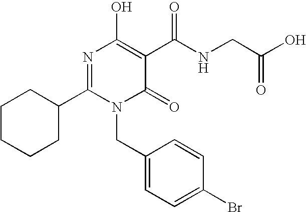

### N-({1-[(4-Bromophenyl)methyl]-2-cyclohexyl-4-hydroxy-6-oxo-1,6-dihydro-5-pyrimidinyl}carbonyl)glycine

A mixture of 4-bromobenzylamine hydrochloride (0.223 g, 1.00 mmol), ethyl cyclohexanecarboximidoate hydrochloride (0.192 g, 1.00 mmol), N,N-diisopropylethylamine (0.348 mL, 2.00 mmol) and 2-methoxyethanol (2 mL) was stirred at room temperature for 18 h, then diethyl malonate (0.303 mL, 2.00 mmol) and 4.37M methanolic sodium methoxide solution (0.915 mL, 4.00 mmol) were added and the mixture refluxed under nitrogen for 18 h. After cooling, water (50 mL) was added and the mixture acidified with 6M aqueous hydrochloric acid (2 mL). The precipitate was filtered, washed with water, dried and triturated with toluene and dried again to give the intermediate, 3-[(4-bromophenyl)methyl]-2-cyclohexyl-6-hydroxy-4(3H)-pyrimidinone (0.203 g, LCMS (ES⁺) m/z 363/365 (MH⁺), as a solid, pure enough for use in the next step. A mixture of the crude pyrimidinone, ethyl 2-isocyanatoacetate (0.123 mL, 1.10 mmol), N,N-diisopropylethylamine (0.192 mL, 1.10 mmol) and dichloromethane (2 mL) was stirred in a microwave reactor at 120° C. for 1 h. After cooling, the solvent was removed under reduced pressure and the residue suspended in ethanol (20 mL). 3 M aqueous sodium hydroxide (4.00 mL, 12.0 mmol) was added dropwise to the suspension at room temperature and the mixture stirred for 3 h, then acidified with 1 M aqueous hydrochloric acid to pH 1-2 and extracted with ethyl acetate. The extracts were dried (MgSO₄) and evaporated under reduced pressure. The residue was purified by HPLC (ODS, 10-90% acetonitrile/water+0.1% trifluoroacetic acid) to give the title compound (0.105 g, 23%). 1H NMR (400 MHz, DMSO-d₆) δ ppm 1.12-1.26 (m, 3 H) 1.38-1.73 (m, 7 H) 2.73-2.88 (m, 1 H) 4.08 (d, J=5.56 Hz, 2 H) 5.35 (s, 2 H) 7.18-7.24 (m, 2 H) 7.50-7.61 (m, 2 H) 9.82 (t, J=5.56 Hz, 1 H) 12.89 (br. s., 1 H) 15.84 (s, 1 H).

### EXAMPLE 15

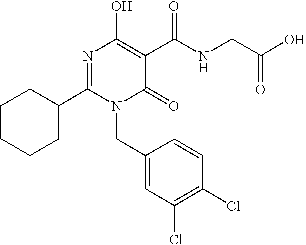

### N-({2-Cyclohexyl-1-[(3,4-dichlorophenyl)methyl]-4-hydroxy-6-oxo-1,6-dihydro-5-pyrimidinyl}carbonyl)glycine

A mixture of 3,4-dichlorobenzylamine (0.133 mL, 1.00 mmol), ethyl cyclohexanecarboximidoate hydrochloride (0.192 g, 1.00 mmol), N,N-diisopropylethylamine (0.174 mL, 1.00 mmol) and 2-methoxyethanol (2 mL) was stirred at room temperature for 18 h, then diethyl malonate (0.303 mL, 2.00 mmol) and 4.37M methanolic sodium methoxide solution (0.686 mL, 3.00 mmol) were added and the mixture refluxed under nitrogen for 18 h. After cooling, water (50 mL) was added and the mixture acidified with 6M aqueous hydrochloric acid (2 mL). The precipitate was filtered, washed with water, dried and triturated with toluene and dried again to give the intermediate, 2-cyclohexyl-3-[(3,4-dichlorophenyl)methyl]-6-hydroxy-4(3H)-pyrimidinone (0.275 g, LCMS (ES^{|}) m/z 353 (MH^{|}), as a solid, pure enough for use in the next step. A mixture of the crude pyrimidinone, ethyl 2-isocyanatoacetate (0.171 mL, 1.53 mmol), N,N-diisopropylethylamine (0.266 mL, 1.53 mmol) and dichloromethane (2 mL) was stirred in a microwave reactor at 120° C. for 1 h. After cooling, the solvent was removed under reduced pressure and the residue suspended in ethanol (20 mL). 3 M aqueous sodium hydroxide (4.00 mL, 12.0 mmol) was added dropwise to the suspension at room temperature and the mixture stirred for 3 h, then acidified with 1 M aqueous hydrochloric acid to pH 1-2 and extracted with ethyl acetate. The extracts were dried (MgSO₄) and evaporated under reduced pressure. The residue was purified by HPLC (ODS, 10-90% acetonitrile/water+0.1% trifluoroacetic acid) to give the title compound (0.074 g, 16%). 1H NMR (400 MHz, DMSO-d₆) δ ppm 1.09-1.31 (m, 3 H) 1.39-1.73 (m, 7 H) 2.77-2.87 (m, 1 H) 4.08 (d, J=5.56 Hz, 2 H) 5.37 (s, 2 H) 7.20 (dd, J=8.46, 2.15 Hz, 1 H) 7.62 (d, J=8.34 Hz, 1 H) 7.64 (d, J=2.02 Hz, 1 H) 9.79 (t, J=5.56 Hz, 1 H) 12.89 (br. s., 1 H) 15.85 (s, 1 H).

### EXAMPLE 16

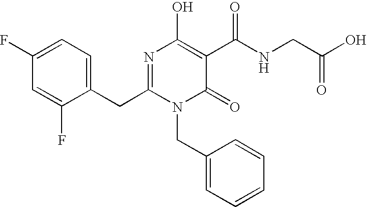

### N-{[2-[(2,4-Difluorophenyl)methyl]-4-hydroxy-6-oxo-1-(phenylmethyl)-1,6-dihydro-5-pyrimidinyl]carbonyl}glycine

16a) Ethyl 2-(2,4-difluorophenyl)ethanimidoate hydrochloride. 2,4-Difluoroacetonitrile (1.97 g, 12.86 mmol) and ethanol (2 mL) was sealed in a flask using a rubber septum and flushed with nitrogen. 4 molar hydrogen chloride in dioxane (5 mL) was added and the mixture was stirred at rt overnight. The solid was diluted with diethyl ether, collected, washed with diethyl ether and dried in vacuo to give the title compound (2.41 g, 80%). 1H NMR (400 MHz, DMSO-d₆) δ ppm 11.90 (br. s., 2 H), 7.55 (dd, 1 H), 7.32 (dd, 1 H), 7.14 (dd, J=8.59 Hz, 1 H), 4.44 (q, J=6.91 Hz, 2 H), 4.11 (s, 2 H), 1.25 (t, J=6.95 Hz, 3 H).

16b) 2-[(2,4-Difluorophenyl)methyl]-1-(phenylmethyl)-4,6(1H,5H)-pyrimidinedione. A mixture of ethyl 2-(2,4-difluorophenyl)ethanimidoate hydrochloride (306 mg, 1.3 mmole), benzylamine (142 uL, 1.3 mmol) and diisopropylethylamine (218 uL, 1.3 mmol) was stirred overnight in methoxyethanol (5 mL). Diethyl malonate (395 uL, 2.6 mmol) followed by sodium ethoxide (1.3 mL of a 3 molar solution in ethanol), the mixture was heated under reflux for 18 hours. The mixture was cooled, diluted with ethyl acetate and washed with 1 molar hydrochloric acid. The aqueous was extracted with ethyl acetate and the combined extracts washed with 1 molar hydrochloric acid, dried and evaporated to a dark oil, (440 mg). Flash chromatography (dichloromethane then ethyl acetate) afforded the title compound (267 mg, 63%). 1H NMR (400 MHz, CHLOROFORM-d) δ ppm 7.31-7.41 (m, 3 H), 7.16 (d, J=6.82 Hz, 3 H), 6.82-6.91 (m, 2 H), 5.81 (s, 1 H), 5.26 (s, 2 H), 4.02 (s, 2 H).

16c) N-{[2-[(2,4-Difluorophenyl)methyl]-4-hydroxy-6-oxo-1-(phenylmethyl)-1,6-dihydro-5-pyrimidinyl]carbonyl}glycine. A mixture of 2-[(2,4-difluorophenyl)methyl]-1-(phenylmethyl)-4,6(1H,5H)-pyrimidinedione (260 mg, 0.79 mmoles), diisopropylethylamine (274 uL, 1.58 mmol) and ethyl isocyanatoacetate (97 uL, 0.87 mmol) in chloroform (10 mL) was sealed in a pressure flask and heated in a microwave reactor (125° C., 1 hour). LCMS indicated incomplete reaction, a second aliquot of ethyl isocyanatoacetate (97 uL, 0.87 mmol) was added and the mixture was heated for a second hour. The mixture was washed with 1 molar hydrochloric acid and evaporated. The residue was taken up in ethanol and 6 molar sodium hydroxide solution (2 mL) was added, then stirred for 3 hours. The mixture was acidified with 1 molar hydrochloric acid to give a solid that was purified by prep. HPLC (30-100% acetonitrile-water-0.1% TFA) to give the title compound (16 mg, 4.7%). 1H NMR (400 MHz, DMSO-d₆) δ ppm 15.90 (s, 1 H), 12.93 (s, 1 H), 9.82 (t, J=5.56 Hz, 1 H), 7.38 (dd, J=7.20 Hz, 2 H), 7.32 (d, 1 H), 7.28 (dd, 1 H), 7.22-7.26 (m, 2 H), 7.19 (dd, 1 H), 7.03 (dd, 1 H), 5.42 (s, 2 H), 4.16 (s, 2 H), 4.08 (d, J=5.31 Hz, 2 H).

### EXAMPLE 17

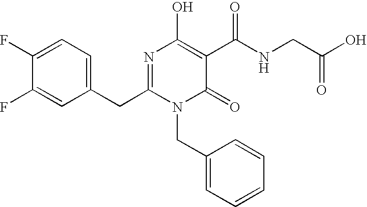

### N-{[2-[(3,4-Difluorophenyl)methyl]-4-hydroxy-6-oxo-1-(phenylmethyl)-1,6-dihydro-5-pyrimidinyl]carbonyl}glycine

17a) Ethyl 2-(3,4-difluorophenyl)ethanimidoate hydrochloride. 3,4-Difluoroacetonitrile (2.44 g, 15.93 mmol) and ethanol (2 mL) was sealed in a flask using a rubber septum and flushed with nitrogen. 4 Molar hydrogen chloride in dioxane (5 mL) was added and the mixture was stirred at rt overnight. The solid was diluted with diethyl ether, collected, washed with diethyl ether and dried in vacuo to give the title compound (2.87 g, 76.5%) 1H NMR (400 MHz, DMSO-d₆) δ ppm 11.75 (br. s., 2 H), 7.37-7.67 (m, 2 H), 7.20-7.31 (m, 1 H), 4.41 (q, J=7.07 Hz, 2 H), 4.05 (s, 2 H), 1.29 (t, J=6.95 Hz, 3 H).

17b) 2-[(3,4-Difluorophenyl)methyl]-1-(phenylmethyl)-4,6(1H,5H)-pyrimidinedione. A mixture of ethyl 2-(3,4-difluorophenyl)ethanimidoate hydrochloride (307 mg, 1.3 mmole), benzylamine (142 uL, 1.3 mmol) and diisopropylethylamine (218 uL, 1.3 mmol) was stirred overnight in methoxyethanol (5 mL). Diethyl malonate (395 uL, 2.6 mmol) followed by sodium ethoxide (1.3 mL of a 3 molar solution in ethanol), the mixture was heated under reflux for 18 hours. The mixture was cooled, diluted with ethyl acetate and washed with 1 molar hydrochloric acid. The aqueous was extracted with ethyl acetate and the combined extracts washed with 1 molar hydrochloric acid, dried and evaporated to a dark oil. Flash chromatography (dichloromethane then ethyl acetate) afforded the title compound (240 mg, 56%). 1H NMR (400 MHz, CHLOROFORM-d) δ ppm 7.40 (s, 1 H), 7.23-7.37 (m, 4 H), 7.05-7.22 (m, 2 H), 6.96 (s, 1 H), 5.77 (s, 1 H), 5.19 (s, 2 H), 4.04 (s, 2 H).

17c) N-{[2-[(3,4-Difluorophenyl)methyl]-4-hydroxy-6-oxo-1-(phenylmethyl)-1,6-dihydro-5-pyrimidinyl]carbonyl}glycine. A mixture of 2-[(3,4-difluorophenyl)methyl]-1-(phenylmethyl)-4,6(1H,5H)-pyrimidinedione (160 mg, 0.44 mmoles), diisopropylethylamine (154 uL, 0.88 mmol) and ethyl isocyanatoacetate (60 uL, 0.53 mmol) in chloroform (10 mL) was sealed in a pressure flask and heated in a microwave reactor (125° C., 1 hour). Additional aliquots of diisopropylethylamine and ethyl isocyanatoacetate were added and the mixture heated for a further 90 minutes at 120° C. The mixture was washed with 1 molar hydrochloric acid and the solvent was evaporated. The residue was taken up in ethanol (2 mL) and 6 molar sodium hydroxide solution (5 mL) was added, then stirred overnight. The mixture was acidified with 1 molar hydrochloric acid to give a solid that was purified by prep. HPLC (30-100% acetonitrile-water-0.1% TFA) and recrystallization from acetic acid-water to give the title compound (30 mg, 16%). 1H NMR (400 MHz, DMSO-d₆) δ ppm 15.91 (s, 1 H), 12.91 (s, 1 H), 9.81 (t, J=5.43 Hz, 1 H), 7.21-7.50 (m, 5 H), 7.17 (d, J=7.07 Hz, 2 H), 6.89-7.07 (m, 1 H), 5.36 (s, 2 H), 4.16 (s, 2 H).

### EXAMPLE 18

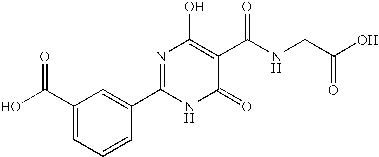

### 3-(5-{[(Carboxymethyl)amino]carbonyl}-4-hydroxy-6-oxo-1,6-dihydro-2-pyrimidinyl)benzoic acid

18a) 3-[Amino(imino)methyl]benzoic acid ammonium salt. A mixture of 3-cyanobenzoic acid (1.47 g, 10.0 mmol), methanol (0.810 mL, 20.0 mmol) and 4M hydrogen chloride in dioxane (20.0 mL, 80.0 mmol) was allowed to stand for 72 h at room temperature. Ether (100 mL) was added and the precipitate filtered, washed with ether and dried. The solid was slurried in methanol (15 mL) and 7M methanolic ammonia solution (20 mL, 140 mmol) added. The mixture was stirred at room temperature 18 h, then the precipitate filtered and dried to give the title compound (1.19 g, 66%) as a solid. 1H NMR (400 MHz, DMSO-d₆+TFA) δ ppm 7.76 (t, J=7.83 Hz, 1 H) 8.03 (m, 1 H) 8.23-8.29 (m, 1 H) 8.33-8.38 (m, 1 H) 9.19 (s, 2 H) 9.45 (s, 2 H).

18b) 3-(4-Hydroxy-6-oxo-1,6-dihydro-2-pyrimidinyl)benzoic acid. A mixture of 3-[amino(imino)methyl]benzoic acid, ammonium salt (1.19 g, 6.57 mmol), diethyl malonate (2.10 g, 13.1 mmol), 4.37M methanolic sodium methoxide solution (3.00 mL, 13.1 mmol) and 2-methoxyethanol (30 mL) was stirred at reflux under nitrogen for 18 h, then cooled and diluted with water (80 mL). 6M aqueous hydrochloric acid (4 mL) was added and the mixture poured into iced water (100 mL). After 1 h, the precipitate was filtered, washed with water and dried to give the title compound (1.47 g, 96%) as a cream solid. 1H NMR (400 MHz, DMSO-d₆) δ ppm 5.44 (s, 1 H) 7.65 (t, J=7.71 Hz, 1 H) 8.07-8.13 (m, 1 H) 8.34 (d, J=7.83 Hz, 1 H) 8.75 (s, 1 H) 12.44 (br. s., 3 H).

18c) Methyl 3-(4-hydroxy-6-oxo-1,6-dihydro-2-pyrimidinyl)benzoate. A mixture of 3-(4-hydroxy-6-oxo-1,6-dihydro-2-pyrimidinyl)benzoic acid (0.335 g, 1.44 mmol), concentrated sulfuric acid (0.5 mL) and methanol (15 mL) was stirred at reflux under nitrogen for 3 h, then cooled and diluted with water (100 mL). After initially neutralizing with sodium bicarbonate, the pH was adjusted to 1 with 6M aqueous hydrochloric acid and the mixture extracted with ethyl acetate. The extracts were dried (MgSO₄) and evaporated under reduced pressure. The residue was chromatographed (silica gel, 5-10% methanol/dichloromethane+0.5% acetic acid) to give the title compound (0.040 g, 11%) as a solid. 1H NMR (400 MHz, DMSO-d₆) δ ppm 3.91 (s, 3 H) 5.46 (s, 1 H) 7.68 (t, J=7.83 Hz, 1 H) 8.11-8.16 (m, 1 H) 8.38 (d, J=8.08 Hz, 1 H) 8.76 (s, 1 H) 11.80 (br. s., 2 H).

18d) 3-(5-{[(Carboxymethyl)amino]carbonyl}-4-hydroxy-6-oxo-1,6-dihydro-2-pyrimidinyl)benzoic acid. A mixture of methyl 3-(4-hydroxy-6-oxo-1,6-dihydro-2-pyrimidinyl)benzoate (0.040 g, 0.162 mmol), ethyl 2-isocyanatoacetate (0.073 mL, 0.650 mmol), N,N-diisopropylethylamine (0.113 mL, 0.650 mmol) and dioxane (2 mL) was stirred in a microwave reactor at 180° C. for 1 h. A further portion each of ethyl 2-isocyanatoacetate (0.073 mL, 0.650 mmol), and N,N-diisopropylethylamine (0.113 mL, 0.650 mmol) was added and the mixture microwaved again at 180° C. for 1 h. After cooling, acetic acid (0.140 mL, 2.45 mmol) was added and the mixture chromatographed (silica gel, 1-8% methanol dichloromethane) to give the intermediate ester. 2 M aqueous sodium hydroxide (4.50 mL, 9.00 mmol) was added dropwise to a stirred solution of the intermediate ester in ethanol (10 mL) at room temperature and the mixture stirred for 18 h. After concentrating under reduced pressure to about 5 mL and filtering, the mixture was acidified to pH 2 with 6M aqueous hydrochloric acid. The precipitate was filtered, washed with water and dried to give the title compound (0.012 g, 22%) as an orange solid. 1H NMR (400 MHz, DMSO-d₆) δ ppm 4.11 (d, J=5.81 Hz, 2 H) 7.70 (t, J=7.83 Hz, 1 H) 8.16-8.24 (m, 1 H) 8.35-8.42 (m, 1 H) 8.68-8.80 (m, 1 H) 9.89 (t, J=5.56 Hz, 1 H) 13.19 (br. s., 3 H) 15.88 (br. s., 1 H)

### EXAMPLE 19

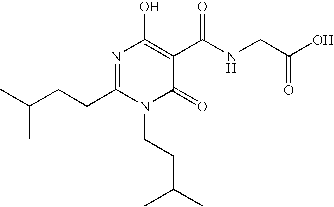

### N-{[4-Hydroxy-1,2-bis(3-methylbutyl)-6-oxo-1,6-dihydro-5-pyrimidinyl]carbonyl}glycine

19a) Ethyl 2-(3,4-difluorophenyl)ethanimidoate hydrochloride. Isocapronitrile (1.46 g, 15.0 mmol) and ethanol (3 mL) were sealed in a flask using a rubber septum and flushed with nitrogen. 4 Molar hydrogen chloride in dioxane (5 mL) was added and the mixture was stirred at rt for 60 hours. The mixture was diluted to low volume, the residue was slurried in diethyl ether, collected, washed with diethyl ether and dried in vacuo to give the title compound (1.0 g, 37%) 1H NMR (400 MHz, DMSO-d₆) δ ppm 12.02 (br. s., 1 H), 11.26 (br. s., 1 H), 4.42 (q, J=7.07 Hz, 2 H), 2.54-2.75 (m, 2 H), 1.41-1.69 (m, 3 H), 1.33 (t, J=7.07 Hz, 3 H), 0.87 (d, J=6.06 Hz, 6 H).

19b) 6-Hydroxy-2,3-bis(3-methylbutyl)-4(3H)-pyrimidinone. A mixture of ethyl 2-(3,4-difluorophenyl)ethanimidoate hydrochloride (500 mg, 2.77 mmoles), isoamylamine (322 uL, 2.77 mmol) and diisopropylethylamine (465 uL, 2.77 mmol) was stirred at rt for 2 hours in methoxyethanol (5 mL). Diethyl malonate (850 uL, 5.6 mmol) followed by sodium ethoxide (2.8 mL of a 3 molar solution in ethanol), were added and the mixture heated under reflux for 2 hours. The mixture was cooled, diluted with ethyl acetate and washed with 1 molar hydrochloric acid. The aqueous was extracted with ethyl acetate and the combined extracts washed with 1 molar hydrochloric acid, dried and evaporated to a solid (660 mg, 94%). 1H NMR (400 MHz, CHLOROFORM-d) δ ppm 5.57 (s, 1 H), 3.92-4.07 (m, 2 H), 2.81-2.98 (m, 2 H), 1.77 (ddd, J=19.14, 12.95, 6.82 Hz, 2 H), 1.64-1.73 (m, 2 H), 1.54-1.64 (m, 2 H), 1.01 (dd, J=6.57, 4.04 Hz, 12 H).

19c) N-{[4-Hydroxy-1,2-bis(3-methylbutyl)-6-oxo-1,6-dihydro-5-pyrimidinyl]carbonyl}glycine. A mixture of 6-Hydroxy-2,3-bis(3-methylbutyl)-4(3H)-pyrimidinone (660 mg, 2.6 mmoles), diisopropylethylamine (900 uL, 5.2 mmol) and ethyl isocyanatoacetate (583 uL, 5.2 mmol) in chloroform (6 mL) was sealed in a pressure flask and heated in a microwave reactor (120° C., 1 hour). The mixture was washed with 1 molar hydrochloric acid and evaporated, the residue was taken up in ethanol (2 mL) and 6 molar sodium hydroxide solution (5 mL) was added, then stirred overnight. The mixture was diluted with 1 molar hydrochloric acid and extracted into ethyl acetate. The organic solution was washed with 1 molar hydrochloric acid and evaporated. The residue was purified by prep. HPLC (30-100% acetonitrile-water-0.1% TFA) to give the title compound (86 mg, 9%) 1H NMR (400 MHz, DMSO-d₆) δ ppm 15.66 (s, 1 H), 12.88 (br. s., 1 H), 9.87 (t, J=5.68 Hz, 1 H), 4.07 (d, J=5.81 Hz, 2 H), 3.92-4.02 (m, 2 H), 2.71-2.91 (m, 2 H), 1.68 (dd, J=13.33, 6.44 Hz, 2 H), 1.56-1.63 (m, 2 H), 1.43-1.55 (m, 2 H), 0.95 (d, 12 H).

### EXAMPLE 20

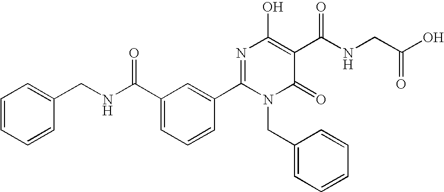

### N-{[4-Hydroxy-6-oxo-1-(phenylmethyl)-2-(3-{[(phenylmethyl)amino]carbonyl}phenyl)-1,6-dihydro-5-pyrimidinyl]carbonyl}glycine

20a) 3-[4-Hydroxy-6-oxo-1-(phenylmethyl)-1,6-dihydro-2-pyrimidinyl]-N-(phenylmethyl)benzamide. A 1 M solution of dimethylaluminium chloride in hexane (10.0 mL, 10.0 mmol) was added dropwise to a stirred solution of benzylamine (1.09 g, 10.0 mmol) in toluene (20 mL) under nitrogen. After stirring at ambient temperature for 30 min, 3-cyanobenzoic acid (0.736 g, 5.00 mmol) was added and the mixture refluxed for 4 h under nitrogen. After cooling, water (3.6 mL) was added, followed by 10% methanol/ethyl acetate (60 mL) and excess sodium bicarbonate. After 0.5 h stirring, the mixture was filtered, the cake washed with ethyl acetate and the combined filtrates evaporated under reduced pressure. The residue was triturated with ethyl acetate and dried to leave a solid. A mixture of the solid, diethyl malonate (1.12 g, 7.00 mmol), 4.37 M sodium methoxide in methanol (1.60 mL, 7.00 mmol) and 2-methoxyethanol (15 mL) was refluxed under nitrogen for 20 h, then cooled and poured into water (150 mL). The mixture was adjusted to pH 1-2 with 6 M aqueous hydrochloric acid, then extracted with ethyl acetate. The extracts were dried (MgSO₄) and evaporated under reduced pressure. The residue was chromatographed (silica gel, 3-10% methanol/dichloromethane) to give the title compound (0.263 g, 13%) as a white solid. 1H NMR (400 MHz, DMSO-d₆) δ ppm 4.46 (d, J=5.81 Hz, 2 H) 5.07 (s, 2 H) 5.52 (s, 1 H) 6.81-6.88 (m, 2 H) 7.14-7.39 (m, 8 H) 7.47-7.56 (m, 2 H) 7.93 (s, 1 H) 7.96-8.03 (m, 1 H) 9.04 (t, J=5.94 Hz, 1 H) 11.76 (br. s., 1 H).

20b) N-{[4-Hydroxy-6-oxo-1-(phenylmethyl)-2-(3-{[(phenylmethyl)amino]carbonyl}phenyl)-1,6-dihydro-5-pyrimidinyl]carbonyl}glycine. A mixture of 3-[4-hydroxy-6-oxo-1-(phenylmethyl)-1,6-dihydro-2-pyrimidinyl]-N-(phenylmethyl)benzamide (0.260 g, 0.632 mmol), ethyl 2-isocyanatoacetate (0.144 mL, 1.28 mmol), N,N-diisopropylethylamine (0.233 mL, 1.28 mmol) and dichloromethane (2 mL) was stirred in a microwave reactor at 120° C. for 1 h, then cooled. Acetic acid (0.153 mL, 2.60 mmol) was added and the mixture chromatographed (silica gel, 1-10% methanol/dichloromethane) to give the intermediate ester. 1 M aqueous sodium hydroxide (2.50 mL, 2.50 mmol) was added dropwise to an ice-cooled, stirred suspension of the intermediate ester in ethanol (10 mL) and the mixture stirred for 3 h. Three mL of this mixture was diluted with water (50 mL) and acidified to pH 1 with 1M aqueous hydrochloric acid. The precipitate was filtered, washed with water and dried to give the title compound (0.037 g, 65%) as a white powder. 1H NMR (400 MHz, DMSO-d₆) δ ppm 4.10 (d, J=5.56 Hz, 2 H) 4.47 (d, J=6.06 Hz, 2 H) 5.13 (s, 2 H) 6.94-7.02 (m, 2 H) 7.17-7.38 (m, 8 H) 7.55 (t, J=7.71 Hz, 1 H) 7.58-7.65 (m, 1 H) 7.95-8.06 (m, 2 H) 9.10 (t, J=5.94 Hz, 1 H) 9.85 (t, J=5.56 Hz, 1 H) 12.92 (br. s., 1 H) 16.08 (s, 1 H).

### EXAMPLE 21

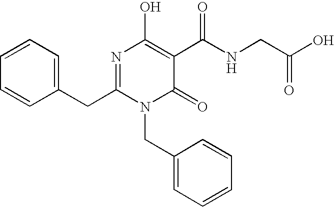

### N-{[4-Hydroxy-6-oxo-1,2-bis(phenylmethyl)-1,6-dihydro-5-pyrimidinyl]carbonyl}glycine

21a) 2-Phenyl-N-(phenylmethyl)ethanimidamide hydrochloride. Benzylamine (2.14 g, 20 mmoles) in toluene (10 mL) was treated with HCl in dioxane and evaporated to leave the hydrochloride salt. Toluene (50 mL) was added and the mixture was stirred under nitrogen and treated with trimethyl aluminum (10 mL of a 2.0 molar solution in toluene). The mixture cleared on addition and was stirred for 30 minutes and benzyl cyanide (2.3 mL, 20 mmoles) was added. The mixture was stirred at 80° C. for 2 hours, cooled and added to a slurry of silica gel in chloroform. The mixture was stirred for 5 minutes, filtered and the filter bed washed with chloroform then methanol. The filtrate was evaporated to an oil which gave a solid on trituration with ether-hexane. (2.85 g, 63%) 1H NMR (400 MHz, DMSO-d₆) δ ppm 10.45 (br. s., 1 H), 9.60 (br. s., 1 H), 8.90 (br. s., 1 H), 7.51 (d, J=7.07 Hz, 2 H), 7.14-7.44 (m, 9 H), 4.52 (s, 2 H), 3.84 (s, 2 H).

21b) 6-Hydroxy-2,3-bis(phenylmethyl)-4(3H)-pyrimidinone. 2-Phenyl-N-(phenylmethyl)ethanimidamide hydrochloride (2.85 g, 10.96 mmoles), diethyl malonate (3.83 mL, 25.4 mmoles) and sodium ethoxide (12.7 mL of a 3.0 molar solution in ethanol) were heated together under reflux in methoxyethanol (50 mL) for 18 hours. The mixture was diluted with ethyl acetate, washed with 1 molar hydrochloric acid and the aqueous layer extracted with ethyl acetate. The combined organic solutions were washed with 1 molar hydrochloric acid and brine, then evaporated onto silica gel and purified by flash chromatography (0-100% ethyl acetate in hexane, then 10% methanol in dichloromethane) to give the title compound on crystallization from ethyl acetate-diethyl ether (1.27 g, 39%) 1H NMR (400 MHz, DMSO-d₆) δ ppm 11.52 (br. s., 1 H), 7.19-7.46 (m, 6 H), 7.03-7.20 (m, 4 H), 5.41 (s, 1 H), 5.18 (s, 2 H), 3.98 (s, 2 H).

21c) N-{[4-Hydroxy-6-oxo-1,2-bis(phenylmethyl)-1,6-dihydro-5-pyrimidinyl]carbonyl}glycine. A mixture of 6-hydroxy-2,3-bis(phenylmethyl)-4(3H)-pyrimidinone (1.27 g, 4.34 mmoles), diisopropylethylamine (2.90 mL, 17.37 mmol) and ethyl isocyanatoacetate (940 uL, 8.68 mmol) in chloroform (10 mL) was sealed in a pressure flask and heated in a microwave reactor (120° C., 80 minutes). The mixture was diluted with dichloromethane, washed with 1 molar hydrochloric acid (×2) and evaporated onto silica gel. Flash chromatography (dichloromethane to 3% methanol in dichloromethane) gave the pure ester, plus some mixed fractions. The pure ester was taken up in ethanol (5 mL) and 6 molar sodium hydroxide solution (5 mL) was added, then stirred for 3 hours. The mixture was acidified with 1 molar hydrochloric acid and extracted with ethyl acetate (×2). The organic solution was washed with 1 molar hydrochloric acid and evaporated to a solid. The solid was slurried in diethyl ether, collected and washed with diethyl ether and hexane to give the title compound (700 mg, 41%) 1H NMR (400 MHz, DMSO-d₆) d ppm 15.91 (s, 1 H), 12.92 (s, 1 H), 9.80 (t, J=5.56 Hz, 1 H), 7.33-7.38 (m, 2 H), 7.24-7.32 (m, 4 H), 7.14-7.21 (m, 4 H), 5.32 (s, 2 H), 4.12 (s, 2 H), 4.07 (d, J=5.81 Hz, 2 H).

### EXAMPLE 22

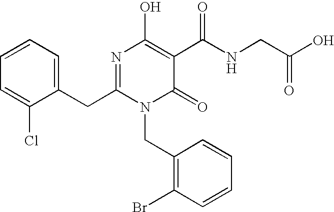

### N-({1-[(2-Bromophenyl)methyl]-2-[(2-chlorophenyl)methyl]-4-hydroxy-6-oxo-1,6-dihydro-5-pyrimidinyl}carbonyl)glycine

22a) N-[(2-Bromophenyl)methyl]-2-(2-chlorophenyl)ethanimidamide hydrochloride. Trimethylaluminum (8.7 mL of a 2 molar solution in toluene) was added to a stirred suspension of 2-bromobenzylamine hydrochloride (3.86 g, 17.34 mmoles) in toluene (25 mL). The mixture was stirred for 2 hours, during which time the reaction produced an exotherm, cleared, and then formed a precipitate. A solution of 2-chlorobenzylcyanide (2.63 g, 17.34 mmoles) in toluene (25 mL) was added and the mixture was heated at 80° C. for 2 hours. The mixture was cooled; some silica gel and chloroform (100 mL) were added then stirred for 5 minutes. The mixture was filtered and washed through with chloroform (100 mL0 and methanol (200 mL). The filtrate was evaporated to a semi-solid that was triturated with diethyl ether to give the title compound (4.1 g, 63%) 1H NMR (400 MHz, DMSO-d₆) δ ppm 8.91 (br. s., 2 H), 7.66 (dd, J=7.58, 1.52 Hz, 2 H), 7.41-7.49 (m, 4 H), 7.28-7.34 (m, 2 H), 4.62 (s, 2 H), 4.09 (s, 2 H).

22b) 3-[(2-Bromophenyl)methyl]-2-[(2-chlorophenyl)methyl]-6-hydroxy-4(3H)-pyrimidinone. N-[(2-Bromophenyl)methyl]-2-(2-chlorophenyl)ethanimidamide hydrochloride (2.0 g, 5.34 mmoles), diethyl malonate (1.80 mL, 11.84 mmoles) and sodium ethoxide (5.92 mL of a 3.0 molar solution in ethanol) were heated together under reflux in methoxyethanol (25 mL) for 24 hours. The mixture was diluted with ethyl acetate, washed with 1 molar hydrochloric acid and the aqueous extracted with ethyl acetate. The combined organic solutions were washed with 1 molar hydrochloric acid and brine, then evaporated onto silica gel and purified by flash chromatography (ethyl acetate-hexane, 0-100%) to give the title compound (650 mg, 30%). 1H NMR (400 MHz, DMSO-d₆) δ ppm 11.43 (s, 1 H), 7.67 (dd, J=7.96, 0.88 Hz, 1 H), 7.31-7.43 (m, 2 H), 7.20-7.31 (m, 4 H), 6.76 (dd, J=7.71, 1.14 Hz, 1 H), 5.44 (s, 1 H), 5.22 (s, 2 H), 4.08 (s, 2 H).

22c) N-({1-[(2-Bromophenyl)methyl]-2-[(2-chlorophenyl)methyl]-4-hydroxy-6-oxo-1,6-dihydro-5-pyrimidinyl}carbonyl)glycine. A mixture of 3-[(2-bromophenyl)methyl]-2-[(2-chlorophenyl)methyl]-6-hydroxy-4(3H)-pyrimidinone (645 mg, 1.59 mmoles), diisopropylethylamine (1.07 mL, 6.36 mmol) and ethyl isocyanatoacetate (675 uL, 6.2 mmol) in chloroform (10 mL) was sealed in a pressure flask and heated in a microwave reactor (120° C., 80 minutes). The mixture was washed with 1 molar hydrochloric acid and evaporated, the residue was taken up in ethanol (2 mL) and 6 molar sodium hydroxide solution (5 mL) was added, then stirred for 2 hours. The mixture was diluted with 1 molar hydrochloric acid and extracted into ethyl acetate. The organic solution was washed with 1 molar hydrochloric acid and evaporated to a foam. Crystallization from ethanol-water, followed by recrystallization from acetic acid-water, with charcoal treatment, gave the title compound (240 mg, 30%) 1H NMR (400 MHz, DMSO-d₆) δ ppm 15.96 (s, 1 H), 12.92 (s, 1 H), 9.74 (t, J=5.56 Hz, 1 H), 7.71 (dd, J=7.83, 1.00 Hz, 1 H), 7.38-7.42 (m, 1 H), 7.25-7.37 (m, 5 H), 6.93 (d, J=7.58 Hz, 1 H), 5.34 (s, 2 H), 4.23 (s, 2 H), 4.06 (d, J=5.56 Hz, 2 H).

### EXAMPLE 23

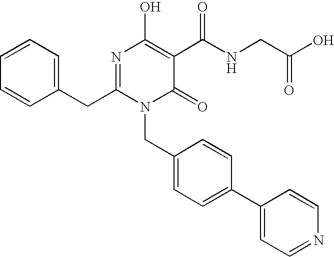

### N-[(4-Hydroxy-6-oxo-2-(phenylmethyl)-1-{[4-(4-pyridinyl)phenyl]methyl}-1,6-dihydro-5-pyrimidinyl)carbonyl]glycine

A mixture of ethyl N-{[1-[(4-bromophenyl)methyl]-4-hydroxy-6-oxo-2-(phenylmethyl)-1,6-dihydro-5-pyrimidinyl]carbonyl}glycinate (see example 24(c), 500 mg, 1.0 mmoles), tetrakis(triphenylphosphine)palladium(0) (58 mg, 0.05 mmoles), 4-pyridinoboronic acid (148 mg, 1.2 mmoles) and potassium carbonate (415 mg, 0.3 mmoles) in water (2.0 mL) and dioxane (10 mL) was heated under reflux for 1 hour. The mixture was cooled and acidified, then extracted with ethyl acetate, which only gave a small amount of product. The aqueous was neutralized and extracted again with ethyl acetate. The combined extracts were evaporated and the residue purified by flash chromatography (dichloromethane to 3% methanol in dichloromethane). The ester was dissolved in ethanol and treated with 6 molar sodium hydroxide solution. A solid was obtained that was collected then washed successively with ethanol, diethyl ether and hexane to afford the title compound as the disodium salt (170 mg, 33%). 1H NMR (400 MHz, DMSO-d₆) δ ppm 10.25 (t, J=4.17 Hz, 1 H), 8.51-8.69 (m, 2 H), 7.76 (d, J=8.34 Hz, 2 H), 7.68 (dd, J=4.55, 1.52 Hz, 2 H), 7.16-7.34 (m, 7 H), 5.08 (br. s., 2 H), 3.76 (s, 2 H), 3.47 (d, J=4.04 Hz, 2 H).

### EXAMPLE 24

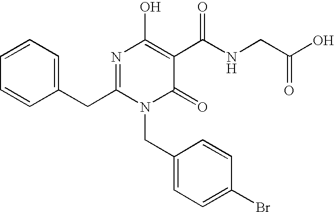

### N-{[1-[(4-Bromophenyl)methyl]-4-hydroxy-6-oxo-2-(phenylmethyl)-1,6-dihydro-5-pyrimidinyl]carbonyl}glycine

24a) N-[(4-Bromophenyl)methyl]-2-phenylethanimidamide hydrochloride. Trimethylaluminium (6.0 mL of a 2 molar solution in toluene) was added to a stirred suspension of 4-bromobenzylamine hydrochloride (2.67 g, 12.0 mmoles) in toluene (15 mL) under nitrogen. The mixture was stirred for 1 hour, then a solution of benzylcyanide (1.38 mL, 12.0 mmoles) in toluene (15 mL) was added and the mixture was heated at 80° C. for 2 hours. The mixture was cooled, poured onto silica gel and chloroform (100 mL) and stirred for 5 minutes. The mixture was filtered and washed through with chloroform and then methanol. The filtrate was evaporated and triturated with diethyl ether-hexane, collected and washed with hexane and dried to give the title compound (2.86 g, 70%) 1H NMR (400 MHz, DMSO-d₆) δ ppm 10.46 (s, 1 H), 9.65 (s, 1 H), 8.50 (s, 1 H), 7.53-7.60 (m, 2 H), 7.44-7.49 (m, 2 H), 7.35-7.41 (m, 2 H), 7.31-7.35 (m, 1 H), 7.29 (d, J=8.34 Hz, 2 H), 4.50 (s, 2 H), 3.83 (s, 2 H).

24b) 3-[(4-Bromophenyl)methyl]-6-hydroxy-2-(phenylmethyl)-4(3H)-pyrimidinone. N-[(4-bromophenyl)methyl]-2-phenylethanimidamide hydrochloride (2.85 g, 8.4 mmoles), diethyl malonate (2.86 mL, 18.8 mmoles) and sodium ethoxide (9.4 mL of a 3.0 molar solution in ethanol) were heated together under reflux in methoxyethanol (50 mL) over the weekend. The mixture was acidified with 1 molar hydrochloric acid and extracted with ethyl acetate (×2). The combined organic solutions were washed with 1 molar hydrochloric acid and evaporated onto silica gel and purified by flash chromatography (dichloromethane to 5% methanol in dichloromethane) a solid was obtained on evaporation of the fractions which was slurried in diethyl ether-hexane, collected and washed with hexane to give the title compound (864 mg, 28%) 1H NMR (400 MHz, DMSO-d₆) δ ppm 7.45-7.53 (m, 2 H), 7.20-7.31 (m, 3 H), 7.15 (d, J=6.82 Hz, 2 H), 7.05 (d, J=8.59 Hz, 2 H), 5.41 (s, 1 H), 5.14 (s, 2 H), 3.99 (s, 2 H).

24c) N-{[1-[(4-Bromophenyl)methyl]-4-hydroxy-6-oxo-2-(phenylmethyl)-1,6-dihydro-5-pyrimidinyl]carbonyl}glycine. A mixture of 3-[(4-bromophenyl)methyl]-6-hydroxy-2-(phenylmethyl)-4(3H)-pyrimidinone (864 mg, 2.33 mmoles), diisopropylethylamine (1.56 mL, 9.32 mmol) and ethyl isocyanatoacetate (507 uL, 4.66 mmol) in chloroform (10 mL) was sealed in a pressure flask and heated in a microwave reactor (120° C., 80 minutes). The mixture was washed with 1 molar hydrochloric acid (×2), dried and evaporated to a solid that was triturated in diethyl ether, collected and dried to give ethyl N-{[1-[(4-bromophenyl)methyl]-4-hydroxy-6-oxo-2-(phenylmethyl)-1,6-dihydro-5-pyrimidinyl]carbonyl}glycinate (900 mg, 81%) as a solid. The ester (400 mg, 0.8 mmoles) was taken up in ethanol (5 mL) and 6 molar sodium hydroxide solution (5 mL) was added, then stirred for 3 hours to give a solid that was collected, washed with ethanol-water, ethanol, diethyl ether and hexane to afford the title compound as the disodium salt, (296 mg, 71%) 1HNMR (400 MHz, DMSO-d₆) δ ppm 10.19 (t, J=4.17 Hz, 1 H), 7.50 (d, J=8.34 Hz, 2 H), 7.29 (dd, J=7.33 Hz, 2 H), 7.19-7.25 (m, 1 H), 7.17 (d, J=6.82 Hz, 2 H), 7.06 (d, J=8.34 Hz, 2 H), 4.98 (s, 2 H), 3.72 (s, 2 H), 3.45 (d, J=4.04 Hz, 2 H).

### EXAMPLE 25

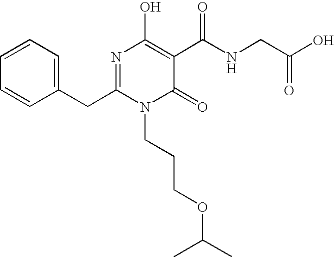

### N-{[4-Hydroxy-1-{3-[(1-methylethyl)oxy]propyl}-6-oxo-2-(phenylmethyl)-1,6-dihydro-5-pyrimidinyl]carbonyl}glycine

25a) N-{3-[(1-Methylethyl)oxy]propyl}-2-phenylethanimidamide hydrochloride. A solution of 3-isopropoxypropylamine (2.34 g, 20 mmole) was stirred in chloroform and treated with 4 molar hydrogen chloride in dioxane (6.0 mL). The mixture was evaporated to dryness to give the hydrochloride salt. The salt was taken up in toluene (25 mL) and treated with trimethyl aluminum (10 mL of a 2.0 molar solution in toluene) under nitrogen atmosphere. The mixture was stirred for 30 minutes and benzyl cyanide (2.22 mL, 20 mmoles) in toluene (25 mL) was added. The mixture was then heated at 80° C. for 2 hours, poured onto a stirred suspension of silica gel in chloroform and stirred for 5 minutes. The mixture was filtered and the filter bed washed successively with chloroform and methanol. The filtrate was evaporate to an oil (4.6 g, 82%) 1H NMR (400 MHz, DMSO-d₆) δ ppm 8.01 (br. s., 3 H), 7.37-7.44 (m, 2 H), 7.30-7.37 (m, 3 H), 4.05 (s, 2 H), 3.52 (dt, J=18.19, 12.13, 6.06 Hz, 1 H), 3.41 (t, J=6.06 Hz, 2 H), 2.80 (t, 2 H), 1.69-1.84 (m, 2 H), 1.08 (d, J=6.06 Hz, 6 H).

25b) 6-Hydroxy-3-{3-[(1-methylethyl)oxy]propyl}-2-(phenylmethyl)-4(3H)-pyrimidinone. N-{3-[(1-Methylethyl)oxy]propyl}-2-phenylethanimidamide hydrochloride (4.6 g, 16.38 mmoles), diethyl malonate (5.95 mL, 39.2 mmoles) and sodium ethoxide (19.6 mL of a 3.0 molar solution in ethanol) were heated together under reflux in methoxyethanol (80 mL) overnight. The mixture was acidified with 1 molar hydrochloric acid and extracted with ethyl acetate (×2). The combined organic solutions were washed with 1 molar hydrochloric acid and evaporated onto silica gel and purified by flash chromatography (dichloromethane to 4% methanol in dichloromethane) to give the title compound (300 mg, 6%) 1H NMR (400 MHz, CHLOROFORM-d) δ ppm 7.34-7.42 (m, 3 H), 7.29-7.34 (m, 2 H), 5.59 (s, 1 H), 4.28 (s, 2 H), 3.99-4.08 (m, 2 H), 3.61 (dt, J=12.38, 6.57, 6.32 Hz, 1 H), 3.45 (t, J=5.43 Hz, 2 H), 1.88-1.99 (m, 2 H), 1.21 (d, J=6.32 Hz, 6 H).

25c) N-{[4-Hydroxy-1-{3-{[(1-methylethyl)oxy]propyl}-6-oxo-2-(phenylmethyl)-1,6-dihydro-5-pyrimidinyl]carbonyl}glycine. A mixture of 6-hydroxy-3-{3-[(1-methylethyl)oxy]propyl}-2-(phenylmethyl)-4(3H)-pyrimidinone (300 mg, 1.0 mmoles), diisopropylethylamine (670 uL, 4.0 mmol) and ethyl isocyanatoacetate (217 uL, 2.0 mmol) in chloroform (5 mL) was sealed in a pressure flask and heated in a microwave reactor (120° C., 80 minutes). The mixture was acidified with 1 molar hydrochloric acid and extracted with ethyl acetate (×2), the combined extracts were washed with 1 molar hydrochloric acid and evaporated. Flash chromatography (dichloromethane to 5% methanol in dichloromethane) gave the pure ester. The ester was taken up in ethanol (3 mL) and 6 molar sodium hydroxide solution (3 mL) was added, then stirred for 3 hours. The mixture was acidified with 1 molar hydrochloric acid and extracted with ethyl acetate (×2), the combined extracts were washed with 1 molar hydrochloric acid and evaporated to a solid that was slurried in diethyl ether, collected, washed with diethyl ether and hexane to give the title compound (114 mg, 28%) 1H NMR (400 MHz, DMSO-d₆) δ ppm 15.74 (s, 1 H), 12.91 (s, 1 H), 9.85 (t, J=5.56 Hz, 1 H), 7.32-7.39 (m, 2 H), 7.24-7.33 (m, 3 H), 4.24 (s, 2 H), 4.08 (d, J=5.56 Hz, 2 H), 3.96-4.04 (m, 2 H), 3.51 (dt, J=18.38, 12.19, 6.06 Hz, 1 H), 3.39 (t, J=5.81 Hz, 2 H), 1.77 (dt, J=14.46, 6.00, 5.81 Hz, 2 H), 1.07 (d, J=6.06 Hz, 6 H).

### EXAMPLE 26

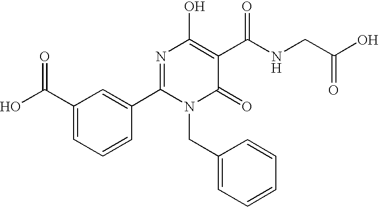

### 3-[5-{[(Carboxymethyl)amino]carbonyl}-4-hydroxy-6-oxo-1-(phenylmethyl)-1,6-dihydro-2-pyrimidinyl]benzoic acid

26a) 3-[4-Hydroxy-6-oxo-1-(phenylmethyl)-1,6-dihydro-2-pyrimidinyl]benzoic acid. A mixture of 3-cyanobenzoic acid (1.47 g, 10.0 mmol), methanol (0.810 mL, 20.0 mmol) and 4M hydrogen chloride in dioxane (20 mL, 80 mmol) was allowed to stand for 5 days. The solid was filtered, washed with ether and dried to leave the crude imidate ester (1.75 g). 1.00 Gram of this material was dissolved in methanol (30 mL) and benzylamine (1.52 mL, 13.9 mmol) was injected. After stirring for 2 h, the solvent was removed under reduced pressure. The residue was dissolved in 2-methoxyethanol (40 mL) and diethyl malonate (3.04 g, 19.0 mmol) and 4.37M methanolic sodium methoxide (4.35 mL, 19.0 mmol) added. The mixture was refluxed under nitrogen for 20 h, cooled and poured into water (300 mL). 6M aqueous hydrochloric acid was added to adjust the pH to 1 and the mixture extracted with ethyl acetate. The extracts were washed with water, brine, dried (MgSO₄) and evaporated under reduced pressure. The residue was chromatographed (silica gel, 20-100% methanol/dichloromethane, then 70-100% methanol/dichloromethane+0.5% acetic acid) to give the title compound (0.106 g, 6%) as a pale yellow solid. LCMS (ES+) m/z 323 (MH⁺).

26b) 3-[5-{[(Carboxymethyl)amino]carbonyl}-4-hydroxy-6-oxo-1-(phenylmethyl)-1,6-dihydro-2-pyrimidinyl]benzoic acid. A mixture of 3-[4-hydroxy-6-oxo-1-(phenylmethyl)-1,6-dihydro-2-pyrimidinyl]benzoic acid (0.104 g, 0.323 mmol), ethyl 2-isocyanatoacetate (0.109 mL, 0.968 mmol), N,N-diisopropylethylamine (0.169 mL, 0.968 mmol) and dichloromethane (2 mL) was stirred in a microwave reactor at 120° C. for 1 h, then cooled. The solvent was removed under reduced pressure and the residue dissolved in ethanol (5 mL). 1 M aqueous sodium hydroxide (2.00 mL, 2.00 mmol) was added dropwise to the stirred solution and the mixture stirred for 18 h at room temperature. The mixture was then diluted with water (40 mL), acidified with 1M aqueous hydrochloric acid, and extracted with ethyl acetate. The extracts were dried (MgSO₄), evaporated under reduced pressure, and the residue purified by HPLC (ODS, 10-90% acetonitrile/water+0.1% trifluoroacetic acid) to give the title compound (0.032 g, 23%) as a cream solid. 1H NMR (400 MHz, DMSO-d₆) δ ppm 4.11 (d, J=5.81 Hz, 2 H) 5.10 (s, 2 H) 6.97-7.02 (m, 2 H) 7.18-7.30 (m, 3 H) 7.57 (t, J=7.83 Hz, 1 H) 7.68-7.72 (m, 1 H) 8.02-8.04 (m, 1 H) 8.05-8.08 (m, 1 H) 9.85 (t, J=5.68 Hz, 1 H) 12.96 (br. s., 1 H) 13.21 (br. s., 1 H) 16.06 (s, 1 H).

### EXAMPLE 27

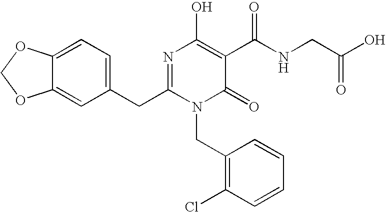

### N-({2-(1,3-Benzodioxol-5-ylmethyl)-1-[(2-chlorophenyl)methyl]-4-hydroxy-6-oxo-1,6-dihydro-5-pyrimidinyl}carbonyl)glycine

27a) 2-(1,3-Benzodioxol-5-yl)-N-[(2-chlorophenyl)methyl]ethanimidamide hydrochloride. A solution of 2-chlorobenzylamine (2.41 g, 20 mmole) was stirred in chloroform and treated with 4 molar hydrogen chloride in dioxane (6.0 mL). The mixture was evaporated to dryness to give the hydrochloride salt. The salt was stirred in toluene (60 mL) and treated with trimethyl aluminum (10 mL of a 2.0 molar solution in toluene) added dropwise under nitrogen. The mixture was stirred for 1 hour and 3,4-methylenedioxybenzyl cyanide (3.22 mL, 20 mmoles) in toluene (20 mL) was added. The mixture was then heated at 80° C. for 2 hours, poured onto a stirred suspension of silica gel in chloroform and stirred for 5 minutes. The mixture was filtered and the filter bed washed successively with chloroform and methanol. The filtrate was evaporate to a solid that was slurried in ether, collected, washed with ether and hexane to give the title compound as a mixture with 2-chlorobenzylamine hydrochloride (5.0 g, 61%) 1H NMR (400 MHz, DMSO-d₆) δ ppm 10.12 (br. s., 1 H), 9.62 (br. s., 1 H), 9.07 (br. s., 1 H), 7.54-7.57 (m, 1 H), 7.37-7.41 (m, 1 H), 7.27-7.37 (m, 2 H), 7.12 (d, J=1.52 Hz, 1 H), 6.98 (dd, 1 H), 6.93 (d, 1 H), 5.96-6.08 (m, 2 H), 6.03 (s, 2H), 4.53 (s, 2 H), 3.75 (s, 2 H).

### 27b) 2-(1,3-Benzodioxol-5-ylmethyl)-3-[(2-chlorophenyl)methyl]-6-hydroxy-4(3H)-pyrimidinone.

Sodium (1.14 g, 49.5 mmole) was dissolved in methoxyethanol (50 mL) under nitrogen. 2-(1,3-benzodioxol-5-yl)-N-[(2-chlorophenyl)methyl]ethanimidamide hydrochloride (4.0 g, 11.8 mmoles) and diethyl malonate (5.01 mL, 33.0 mmoles) were added and the mixture was heated under reflux overnight. The mixture was cooled, diluted with 1 molar hydrochloric acid and extracted with ethyl acetate (×2). The combined extracts washed with 1 molar hydrochloric acid (×2) and evaporated onto silica gel. Flash chromatography (dichloromethane to 5% methanol in dichloromethane) gave the title compound (1.6 g, 26%) 1H NMR (400 MHz, DMSO-d₆) δ ppm 7.45 (dd, J=7.96, 1.14 Hz, 1 H), 7.26 (ddd, J=7.64, 1.64 Hz, 1 H), 7.18 (ddd, J=7.58, 1.26 Hz, 1 H), 6.67-6.76 (m, 2 H), 6.58 (dd, J=7.83, 1.77 Hz, 1 H), 6.53 (dd, J=7.58, 1.26 Hz, 1 H), 5.92 (s, 2 H), 5.41 (s, 1 H), 5.16 (s, 2 H), 3.91 (s, 2 H).

27c) N-({2-(1,3-Benzodioxol-5-ylmethyl)-1-[(2-chlorophenyl)methyl]-4-hydroxy-6-oxo-1,6-dihydro-5-pyrimidinyl}carbonyl)glycine. A mixture of 2-(1,3-benzodioxol-5-ylmethyl)-3-[(2-chlorophenyl)methyl]-6-hydroxy-4(3H)-pyrimidinone (1.60 g, 4.31 mmoles), diisopropylethylamine (2.99 mL, 17.26 mmol) and ethyl isocyanatoacetate (967 uL, 8.62 mmol) in chloroform (10 mL) was sealed in a pressure flask and heated in a microwave reactor (120° C., 80 minutes). The mixture was washed with 1 molar hydrochloric acid (×2) and evaporated onto silica gel. Flash chromatography (hexane to 50% ethyl acetate in hexane) gave the pure ester. The ester was taken up in ethanol (10 mL) and 6 molar sodium hydroxide solution (5.0 mL) was added, then stirred for until hydrolysis complete. The mixture was acidified with 1 molar hydrochloric acid and extracted into ethyl acetate (×2), the combined organic solutions were evaporated and the residue crystallized from acetic acid plus a little water. The solid was collected, washed with acetic acid, diethyl ether and hexane to give the title compound (300 mg, 15%). 1H NMR (400 MHz, DMSO-d₆) δ ppm 15.97 (s, 1 H), 12.92 (s, 1 H), 9.71 (t, J=5.56 Hz, 1 H), 7.48 (dd, J=7.96 1.14 Hz, 1 H), 7.29 (ddd, J=7.71, 1.52 Hz, 1 H), 7.19 (ddd, J=7.58, 1.26 Hz, 1 H), 6.67-6.81 (m, 3 H), 6.63 (dd, J=7.96, 1.64 Hz, 1 H), 5.94 (s, 2 H), 5.28 (s, 2 H), 3.94-4.15 (m, 4 H).

### EXAMPLE 28

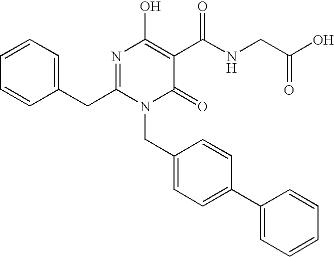

### N-{[1-(4-Biphenylylmethyl)-4-hydroxy-6-oxo-2-(phenylmethyl)-1,6-dihydro-5-pyrimidinyl]carbonyl}glycine

28a) N-(4-Biphenylylmethyl)-2-phenylethanimidamide hydrochloride. A solution of 4-phenylbenzylamine (3.66 g, 20 mmole) was stirred in chloroform and treated with 4 molar hydrogen chloride in dioxane (6.0 mL). The mixture was evaporated to dryness to give the hydrochloride salt. The salt was stirred in toluene (60 mL) and treated with trimethyl aluminum (10 mL of a 2.0 molar solution in toluene) added dropwise under nitrogen. The mixture was stirred for 1 hour and benzyl cyanide (2.22 mL, 20 mmoles) in toluene (20 mL) was added. The mixture was then heated at 80° C. for 2 hours, poured onto a stirred suspension of silica gel in chloroform and stirred for 5 minutes. The mixture was filtered and the filter bed washed successively with chloroform and methanol. The filtrate was evaporate to a solid that was slurried in ether, collected, washed with ether and hexane to give the title compound as a mixture with 4-phenylbenzylamine hydrochloride (4.08 g, 61%) 1H NMR (400 MHz, DMSO-d₆) δ ppm 10.48 (br. s., 1 H), 9.67 (br. s., 1 H), 9.10 (br. s., 1 H), 7.66 (d, J=8.34 Hz, 4 H), 7.50-7.54 (m, 2 H), 7.46-7.50 (m, 3 H), 7.39-7.44 (m, 4 H), 7.33 (ddd, 1 H), 4.56 (s, 2 H), 3.86 (s, 2 H).

28b) 3-(4-Biphenylylmethyl)-6-hydroxy-2-(phenylmethyl)-4(3H)-pyrimidinone. Sodium (985 mg, 40.75 mmole) was dissolved in methoxyethanol (50 mL) under nitrogen. N-(4-biphenylylmethyl)-2-phenylethanimidamide hydrochloride (4.0 g, 11.8 mmoles) and diethyl malonate (4.12 g, 27.2 mmoles) were added and the mixture was heated under reflux for 24 hours. The mixture was cooled, diluted with 1 molar hydrochloric acid and extracted with ethyl acetate (×2). The combined extracts washed with 1 molar hydrochloric acid and evaporated onto silica gel. Flash chromatography (hexane to ethyl acetate) gave the title compound (1.87 g, 43%) 1H NMR (400 MHz, DMSO-d₆) δ ppm 11.52 (br. s., 1 H), 7.63 (t, J=7.45 Hz, 4 H), 7.47 (t, J=7.58 Hz, 2 H), 7.37 (t, J=7.33 Hz, 1 H), 7.31 (t, J=7.20 Hz, 2 H), 7.25 (d, J=7.33 Hz, 1 H), 7.19 (t, J=8.08 Hz, 4 H), 5.43 (s, 1 H), 5.22 (s, 2H), 4.03 (s, 2 H)

28c) N-{[1-(4-Biphenylylmethyl)-4-hydroxy-6-oxo-2-(phenylmethyl)-1,6-dihydro-5-pyrimidinyl]carbonyl glycine. A mixture of 3-(4-biphenylylmethyl)-6-hydroxy-2-(phenylmethyl)-4(3H)-pyrimidinone (1.87 g, 5.0 mmoles), diisopropylethylamine (3.46 mL, 20.0 mmol) and ethyl isocyanatoacetate (1.12 mL, 10.0 mmol) in chloroform (10 mL) was sealed in a pressure flask and heated in a microwave reactor (120° C., 80 minutes). The mixture was washed with 1 molar hydrochloric acid (×2) and evaporated onto silica gel. Flash chromatography (hexane to 50% ethyl acetate in hexane) gave the pure ester as a foam (1.22 g). The ester was taken up in ethanol (10 mL) and 6 molar sodium hydroxide solution (5.0 mL) was added, then stirred for until hydrolysis complete. The solidified mixture was partitioned between 1 molar hydrochloric acid and ethyl acetate, the organic solution was washed with 1 molar hydrochloric acid and evaporated to a solid that was slurried in ethanol, collected, washed with ethanol, diethyl ether and hexane to give the title compound (620 mg, 26%) 1H NMR (400 MHz, DMSO-d₆) δ ppm 15.93 (s, 1 H), 12.92 (s, 1 H), 9.82 (t, J=5.56 Hz, 1 H), 7.55-7.72 (m, 4 H), 7.47 (dd, J=7.58 Hz, 2 H), 7.37 (dd, J=7.36, 1.23 Hz, 1 H), 7.22-7.33 (m, 5 H), 7.17-7.22 (m, 2 H), 5.36 (s, 2 H), 4.17 (s, 2 H), 4.08 (d, J=5.56 Hz, 2 H).

### EXAMPLE 29

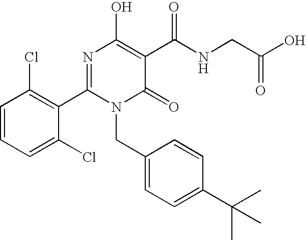

### N-[(2-(2,6-Dichlorophenyl)-1-{[4-(1,1-dimethylethyl)phenyl]methyl}-4-hydroxy-6-oxo-1,6-dihydro-5-pyrimidinyl)carbonyl]glycine

### 29a) 2-(2,6-Dichlorophenyl)-3-{[4-(1,1-dimethylethyl)phenyl]methyl}-6-hydroxy-4(3H)-pyrimidinone.

A 1 M solution of dimethylaluminium chloride in hexane (2.75 mL, 2.75 mmol) was added to a stirred solution of 4-tert-butylbenzylamine (0.408 g, 2.50 mmol) and 2,6-dichlorobenzonitrile (0.860 g, 5.00 mmol) in toluene (15 mL) at room temperature, then the mixture refluxed for 18 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 24 h. After cooling, the mixture was poured into water (200 mL), washed with ether and adjusted to pH 0 with 6 M aqueous hydrochloric acid, then extracted with ethyl acetate. The extracts were washed with water, brine, dried (MgSO₄) and evaporated under reduced pressure. The residue was triturated with ether and dried to give the title compound (0.612 g, 61%) as a cream solid. 1H NMR (400 MHz, DMSO-d₆) δ ppm 1.23 (s, 9 H) 4.87 (s, 2 H) 5.59 (s, 1 H) 6.74-6.78 (m, 2 H) 7.15-7.22 (m, 2 H) 7.52-7.64 (m, 3 H) 11.89 (br. s., 1 H).

29b) N-[(2-(2,6-Dichlorophenyl)-1-{[4-(1,1-dimethylethyl)phenyl]methyl}-4-hydroxy-6-oxo-1,6-dihydro-5-pyrimidinyl)carbonyl]glycine. A mixture of 2-(2,6-dichlorophenyl)-3-{[4-(1,1-dimethylethyl)phenyl]methyl}-6-hydroxy-4(3H)-pyrimidinone (0.555 g, 1.38 mmol), ethyl 2-isocyanatoacetate (0.309 mL, 2.75 mmol), N,N-diisopropylethylamine (0.479 mL, 2.75 mmol) and dichloromethane (5 mL) was stirred in a microwave reactor at 120° C. for 1 h, then cooled. 1 M aqueous hydrochloric acid (10 mL) was added and the mixture extracted with dichloromethane. The extracts were dried (MgSO₄) and evaporated under reduced pressure. The residue was chromatographed (silica gel, 1-5% methanol/dichloromethane) to give the intermediate ester. 1 M aqueous sodium hydroxide (5.00 mL, 5.00 mmol) was added dropwise to a stirred solution of the intermediate ester in ethanol (20 mL) and the mixture stirred for 5 h, then acidified to pH 1 with 1M aqueous hydrochloric acid. Water was added to precipitate a solid. After 10 min, the precipitate was filtered, washed with water and dried to give the title compound (0.315 g, 45%) as a white solid. 1H NMR(400 MHz, DMSO-d₆) δ ppm 1.23 (s, 9 H) 4.13 (d, J=5.81 Hz, 2 H) 4.99 (s, 2 H) 6.81-6.88 (m, 2 H) 7.19-7.28 (m, 2 H) 7.58-7.70 (m, 3 H) 9.87 (t, J=5.68 Hz, 1 H) 12.99 (br. s., 1 H).

### EXAMPLE 30

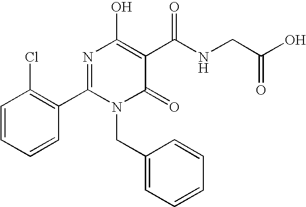

### N-{[2-(2-Chlorophenyl)-4-hydroxy-6-oxo-1-(phenylmethyl)-1,6-dihydro-5-pyrimidinyl]carbonyl}glycine

30a) 2-(2-Chlorophenyl)-6-hydroxy-3-(phenylmethyl)-4(3H)-pyrimidinone. A 1 M solution of dimethylaluminium chloride in hexane (2.75 mL, 2.75 mmol) was added to a stirred solution of benzylamine (0.268 g, 2.50 mmol) and 2-chlorobenzonitrile (0.688 g, 5.00 mmol) in toluene (15 mL) at room temperature, then the mixture refluxed for 4 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into 0.1 M aqueous hydrochloric acid (200 mL) and extracted with ethyl acetate. The extracts were washed with 1 M aqueous sodium carbonate and these extracts washed with ether. After adjusting to pH 1 with 1 M aqueous hydrochloric acid, the mixture was again extracted with ethyl acetate. The extracts were dried (MgSO₄) and evaporated under reduced pressure to give the title compound (0.708 g, 91%) as a solid. 1H NMR (400 MHz, DMSO-d₆) δ ppm 4.68 (d, J=15.66 Hz, 1 H) 5.18 (d, J=15.66 Hz, 1 H) 5.56 (s, 1 H) 6.79-6.88 (m, 2 H) 7.19-7.23 (m, 3 H) 7.33-7.38 (m, 2 H) 7.47-7.60 (m, 2 H) 11.78 (br. s., 1 H).

30b) N-{[2-(2-Chlorophenyl)-4-hydroxy-6-oxo-1-(phenylmethyl)-1,6-dihydro-5-pyrimidinyl]carbonyl}glycine. A mixture of 2-(2-chlorophenyl)-6-hydroxy-3-(phenylmethyl)-4(3H)-pyrimidinone (0.700, 2.24 mmol), ethyl 2-isocyanatoacetate (0.501 mL, 4.48 mmol), N,N-diisopropylethylamine (0.777 mL, 4.48 mmol) and dichloromethane (6 mL) was stirred in a microwave reactor at 120° C. for 1 h, then cooled. 1 M aqueous hydrochloric acid (10 mL) was added and the mixture extracted with dichloromethane. The extracts were dried (MgSO₄) and evaporated under reduced pressure. The residue was chromatographed (silica gel, 1-8% methanol/dichloromethane) to give the intermediate ester. 1 M aqueous sodium hydroxide (4.00 mL, 4.00 mmol) was added dropwise to a stirred solution of the intermediate ester in ethanol (16 mL) and the mixture stirred for 4 h, then acidified with 6M aqueous hydrochloric acid (2 mL). Water was added and the mixture extracted with ethyl acetate. The extracts were dried (MgSO₄) and evaporated under reduced pressure and the residue chromatographed (silica gel, 0.5-10% methanol/dichloromethane+0.5% acetic acid). The product was further purified by HPLC (ODS, 10-90% acetonitrile/water+0.1% trifluoroacetic acid) to give the title compound (0.073 g, 8%) as a foam. 1H NMR (400 MHz, DMSO-d₆) δ ppm 4.12 (d, J=5.81 Hz, 2 H) 4.88 (d, J=15.66 Hz, 1 H) 5.16 (d, J=15.92 Hz, 1 H) 6.90-6.99 (m, 2 H) 7.18-7.28 (m, 3 H) 7.40-7.48 (m, 1 H) 7.49-7.64 (m, 3 H) 9.85 (t, J=5.68 Hz, 1 H) 12.94 (br. s., 1 H) 16.21 (s, 1 H).

30c)

### EXAMPLE 31

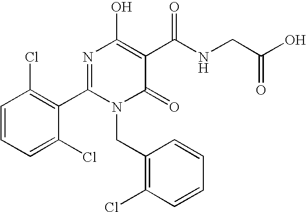

### N-{[1-[(2-Chlorophenyl)methyl]-2-(2,6-dichlorophenyl)-4-hydroxy-6-oxo-1,6-dihydro-5-pyrimidinyl]carbonyl}glycine

31a) 3-[(2-Chlorophenyl)methyl]-2-(2,6-dichlorophenyl)-6-hydroxy-4(3H)-pyrimidinone. A 1 M solution of dimethylaluminium chloride in hexane (2.75 mL, 2.75 mmol) was added to a stirred solution of 2-chlorobenzylamine (0.354 g, 2.50 mmol) and 2,6-dichlorobenzonitrile (0.860 g, 5.00 mmol) in toluene (15 mL) at room temperature, then the mixture refluxed for 18 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL), washed with ether and acidified with 6 M aqueous hydrochloric acid, then extracted with ethyl acetate. The extracts were washed with water, brine, dried (MgSO₄) and evaporated under reduced pressure. The residue was triturated with ether and dried to give the title compound (0.695 g, 73%) as a cream solid. 1H NMR (400 MHz, DMSO-d₆) δ ppm 5.07 (s, 2 H) 5.66 (s, 1 H) 7.03-7.11 (m, 1 H) 7.22-7.37 (m, 3 H) 7.50-7.63 (m, 3 H) 12.05 (s, 1 H),

31b) N-{[1-[(2-Chlorophenyl)methyl]-2-(2,6-dichlorophenyl)-4-hydroxy-6-oxo-1,6-dihydro-5-pyrimidinyl]carbonyl}glycine. A mixture of 3-[(2-chlorophenyl)methyl]-2-(2,6-dichlorophenyl)-6-hydroxy-4(3H)-pyrimidinone (0.691, 1.81 mmol), ethyl 2-isocyanatoacetate (0.406 mL, 3.62 mmol), N,N-diisopropylethylamine (0.631 mL, 3.62 mmol) and dichloromethane (6 mL) was stirred in a microwave reactor at 130° C. for 1 h, then cooled and poured into 1 M aqueous hydrochloric acid (20 mL). The mixture was extracted with dichloromethane and the extracts dried (MgSO₄), then evaporated under reduced pressure. The residue was chromatographed (silica gel, 0.5-5% methanol dichloromethane) to give the intermediate ester. 1 M aqueous sodium hydroxide (6.50 mL, 6.50 mmol) was added dropwise to a stirred solution of the intermediate ester in ethanol (25 mL) and the mixture stirred for 2 h, then acidified with 6M aqueous hydrochloric acid (2 mL). Water (150 mL) was added to precipitate a solid. After stirring 18 h, the precipitate was filtered, washed with water and dried to give the title compound (0.423 g, 48%) as a white solid. 1H NMR (400 MHz, DMSO-d₆) δ ppm 4.14 (d, J=5.81 Hz, 2 H) 5.19 (s, 2 H) 7.04-7.15 (m, 1 H) 7.21-7.38 (m, 3 H) 7.50-7.72 (m, 3 H) 9.85 (t, J=5.56 Hz, 1 H) 13.00 (br. s., 1 H).

### EXAMPLE 32

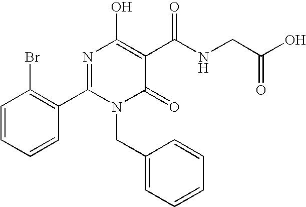

### N-}[2-(2-Bromophenyl)-4-hydroxy-6-oxo-1-(phenylmethyl)-1,6-dihydro-5-pyrimidinyl]carbonyl}glycine

32a) 2-(2-Bromophenyl)-6-hydroxy-3-(phenylmethyl)-4(3H)-pyrimidinone. A 1 M solution of dimethylaluminium chloride in hexane (2.75 mL, 2.75 mmol) was added to a stirred solution of benzylamine (0.268 g, 2.50 mmol) and 2-bromobenzonitrile (0.910 g, 5.00 mmol) in toluene (15 mL) at room temperature, then the mixture refluxed for 18 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 8 h. After cooling, the mixture was poured into water (200 mL), washed with ether and acidified to pH 1 with 6 M aqueous hydrochloric acid, then extracted with ethyl acetate. The extracts were washed with water, brine, dried (MgSO₄) and evaporated under reduced pressure. The residue was chromatographed (silica gel, 2-10% methanol/dichloromethane) to give the title compound (0.759 g, 85%) as a colourless foam. 1H NMR (400 MHz, DMSO-d₆) δ ppm 4.60 (d, J=15.41 Hz, 1 H) 5.23 (d, J=15.41 Hz, 1 H) 5.56 (s, 1 H) 6.83-6.89 (m, 2 H) 7.18-7.23 (m, 3 H) 7.29 (dd, J=7.58, 1.77 Hz, 1 H) 7.38 (td, J=7.52, 1.14 Hz, 1 H) 7.44 (td, J=7.71, 1.77 Hz, 1 H) 7.74 (dd, J=8.08, 1.01 Hz, 1 H) 11.80 (br. s., 1 H).

32b) N-{[2-(2-Bromophenyl)-4-hydroxy-6-oxo-1-(phenylmethyl)-1,6-dihydro-5-pyrimidinyl]carbonyl}glycine di-sodium salt. A mixture of 2-(2-bromophenyl)-6-hydroxy-3-(phenylmethyl)-4(3H)-pyrimidinone (0.740, 2.07 mmol), ethyl 2-isocyanatoacetate (0.465 mL, 4.14 mmol), N,N-diisopropylethylamine (0.721 mL, 4.14 mmol) and dichloromethane (6 mL) was stirred in a microwave reactor at 130° C. for 1 h, then cooled and poured into 1 M aqueous hydrochloric acid (15 mL). The mixture was extracted with dichloromethane and the extracts dried (MgSO₄), then evaporated under reduced pressure. The residue was chromatographed (silica gel, 1-5% methanol dichloromethane) to give the intermediate ester. 1 M aqueous sodium hydroxide (7.50 mL, 7.50 mmol) was added dropwise to a stirred solution of the intermediate ester in ethanol (30 mL) and the mixture stirred for 2.5 h. The precipitate was filtered, washed with ethanol and dried to give the title compound (0.406 g, 39%) as a white solid. 1H NMR (400 MHz, DMSO-d₆) δ ppm 3.51 (d, J=4.04 Hz, 2 H) 4.24 (d, J=16.17 Hz, 1 H) 5.37 (d, J=15.92 Hz, 1 H ) 6.82-6.88 (m, 2 H) 7.00 (d, J=7.58 Hz, 1 H) 7.13-7.22 (m, 3 H) 7.27 (t, J=7.45 Hz, 1 H) 7.31-7.38 (m, 1 H) 7.69 (d, J=8.08 Hz, 1 H) 10.22 (t, J=3.92 Hz, 1 H).

### EXAMPLE 33

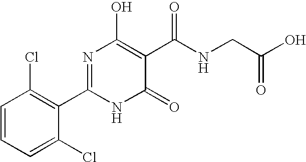

### N-{[2-(2,6-Dichlorophenyl)-4-hydroxy-6-oxo-1,6-dihydro-5-pyrimidinyl]carbonyl}glycine

33a) 2-(2,6-Dichlorophenyl)-6-hydroxy-4(1H)-pyrimidinone. A 2 M solution of trimethylaluminium in hexane (1.38 mL, 2.76 mmol) was added to a stirred mixture of powdered ammonium chloride (0.135 g, 2.52 mmol), 2,6-dichlorobenzonitrile (0.860 g, 5.00 mmol) and toluene (15 mL) at room temperature. After 20 min stirring, the mixture was refluxed for 18 h under nitrogen, then cooled and the solvent removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 6 h. After cooling, the mixture was poured into water (200 mL), washed with ether and acidified to pH 1 with 6 M aqueous hydrochloric acid, then extracted with ethyl acetate. The extracts dried (MgSO₄) and evaporated under reduced pressure. The residue was triturated with ether and the solid collected, washed with ether and dried to give the title compound (0.265 g, 41%) as a cream solid. 1H NMR (400 MHz, DMSO-d₆) δ ppm 5.37 (s, 1 H) 7.56 (dd, J=9.35, 6.57 Hz, 1 H) 7.60-7.65 (m, 2 H) 11.69 (br. s., 1 H) 12.64 (br. s., 1 H).

33b) N-{[2-(2,6-Dichlorophenyl)-4-hydroxy-6-oxo-1,6-dihydro-5-pyrimidinyl]carbonyl}glycine. A mixture of 2-(2,6-dichlorophenyl)-6-hydroxy-4(1H)-pyrimidinone (0.260, 1.01 mmol), ethyl 2-isocyanatoacetate (0.340 mL, 3.03 mmol), N,N-diisopropylethylamine (0.528 mL, 3.03 mmol) and dioxane (5 mL) was stirred in a microwave reactor at 180° C. for 1 h, then cooled. 1 M Aqueous hydrochloric acid (10 mL) was added and the mixture extracted with dichloromethane. The extracts were dried (MgSO₄), evaporated under reduced pressure and the residue chromatographed (silica gel, 1-9% methanol/dichloromethane) to give the intermediate ester. 1 M Aqueous sodium hydroxide (4.00 mL, 4.00 mmol) was added dropwise to a stirred suspension of the intermediate ester in ethanol (15 mL) and the mixture stirred for 3 h. The precipitate was filtered, washed with ethanol and dried. The solid was dissolved in water (10 mL) and the solution acidified to pH 1 with 1 M aqueous hydrochloric acid. The precipitate was filtered, washed with water and dried to give the title compound (0.062 g, 17%) as a white powder. 1H NMR (400 MHz, DMSO-d₆) δ ppm 4.12 (d, J=5.81 Hz, 2 H) 7.62 (dd, J=9.60, 6.32 Hz, 1 H) 7.65-7.70 (m, 2 H) 9.82 (s, 1 H) 12.94 (s, 1 H) 13.66 (s, 1 H) 16.21 (s, 1 H).

### EXAMPLE 34

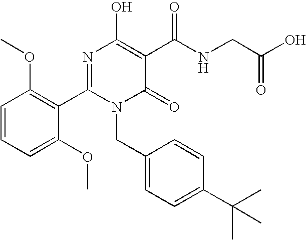

### N-[(2-[2,6-Bis(methyloxy)phenyl]-1-{[4-(1,1-dimethylethyl)phenyl]methyl}-4-hydroxy-6-oxo-1,6-dihydro-5-pyrimidinyl)carbonyl]glycine

34a) 2-[2,6-Bis(methyloxy)phenyl-3-{[4-(1,1-dimethylethyl)phenyl]methyl}-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,6-dimethoxybenzonitrile (0.816 g, 5.00 mmol) and toluene (15 mL) at room temperature, then the mixture refluxed for 18 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL), washed with ether and acidified to pH 1 with 6 M aqueous hydrochloric acid, then extracted with ethyl acetate. The extracts were washed with water, brine, dried (MgSO₄) and evaporated under reduced pressure. The residue triturated with ether and the solid collected, washed with ether and dried to give the title compound (0.528 g, 54%) as a pale yellow powder. 1H NMR (400 MHz, DMSO-d₆) δ ppm 1.22 (s, 9 H) 3.50 (s, 6 H) 4.78 (s, 2 H) 5.46 (br. s., 1 H) 6.68 (d, J=8.34 Hz, 2 H) 6.69-6.72 (m, 2 H) 7.16-7.22 (m, 2 H) 7.44 (t, J=8.46 Hz, 1 H) 11.50 (br. s., 1 H).

34b) N-[(2-[2,6-Bis(methyloxy)phenyl]-1-{[4-(1,1-dimethylethyl)phenyl]methyl}-4-hydroxy-6-oxo-1,6-dihydro-5-pyrimidinyl)carbonyl]glycine. A mixture of 2-[2,6-bis(methyloxy)phenyl]-3-{[4-(1,1-dimethylethyl)phenyl]methyl}-6-hydroxy-4(3H)-pyrimidinone (0.526, 1.33 mmol), ethyl 2-isocyanatoacetate (0.299 mL, 2.66 mmol), N,N-diisopropylethylamine (0.463 mL, 2.66 mmol) and dichloromethane (5 mL) was stirred in a microwave reactor at 130° C. for 1 h, then cooled. 1 M Aqueous hydrochloric acid (10 mL) was added and the mixture extracted with dichloromethane. The extracts were dried (MgSO₄), then evaporated under reduced pressure and the residue chromatographed (silica gel, 1-8% methanol/dichloromethane) to give the intermediate ester. 1 M aqueous sodium hydroxide (5.50 mL, 5.50 mmol) was added dropwise to a stirred solution of the intermediate ester in ethanol (25 mL) and the mixture stirred for 2 h, then acidified to pH 1 with 6M aqueous hydrochloric acid. Water was added to the point of cloudiness and the mixture stirred for 18 h, then slowly diluted with more water (200 mL). After stirring 18 h, the precipitate was filtered, washed with water and dried. The product was further purified by HPLC (ODS, 10-90% acetonitrile/water+0.1% trifluoroacetic acid) to give the title compound (0.132 g, 20%) as a solid. 1H NMR (400 MHz, DMSO-d₆) δ ppm 1.22 (s, 9 H) 3.55 (s, 6 H) 4.11 (d, J=5.56 Hz, 2 H) 4.90 (s, 2 H) 6.72 (d, J=8.59 Hz, 2 H) 6.75-6.80 (m, 2 H) 7.18-7.27 (m, 2 H) 7.48 (t, J=8.46 Hz, 1 H) 9.88 (t, J=5.56 Hz, 1 H) 12.94 (br. s., 1 H) 16.02 (s, 1 H).

### EXAMPLE 35

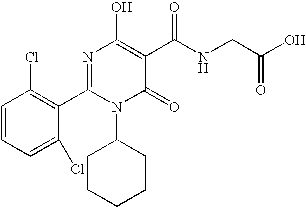

### N-{[1-Cyclohexyl-2-(2,6-dichlorophenyl)-4-hydroxy-6-oxo-1,6-dihydro-5-pyrimidinyl]carbonyl}glycine

35a) 3-Cyclohexyl-2-(2,6-dichlorophenyl)-6-hydroxy-4(3H)-pyrimidinone. A 1 M solution of dimethylaluminium chloride in hexane (2.75 mL, 2.75 mmol) was added to a stirred mixture of cyclohexylamine (0.248 g, 2.50 mmol), 2,6-dichlorobenzonitrile (0.860 g, 5.00 mmol) and toluene (15 mL) at room temperature, then the mixture refluxed for 7 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL), washed with ether and acidified to pH 1 with 6 M aqueous hydrochloric acid, then extracted with ethyl acetate. The extracts were washed with water, brine, dried (MgSO₄) and evaporated under reduced pressure. The residue triturated with ether and the solid collected, washed with ether and dried to give the title compound (0.126 g, 15%) as a white solid. 1 H NMR (400 MHz, DMSO-d₆) δ ppm 0.82-0.96 (m, 2 H) 0.98-1.13 (m, 1 H) 1.44-1.53 (m, 1 H) 1.67-1.78 (m, 4 H) 2.53-2.60 (m, 2 H) 3.30-3.38 (m, 1 H) 5.42 (s, 1 H) 7.63 (dd, J=8.84, 7.07 Hz, 1 H) 7.68-7.75 (m, 2 H) 11.72 (br. s., 1 H).

35b) N-{[1-Cyclohexyl-2-(2,6-dichlorophenyl)-4-hydroxy-6-oxo-1,6-dihydro-5-pyrimidinyl]carbonyl}glycine. A mixture of 3-cyclohexyl-2-(2,6-dichlorophenyl)-6-hydroxy-4(3H)-pyrimidinone (0.124, 0.366 mmol), ethyl 2-isocyanatoacetate (0.082 mL, 0.732 mmol), N,N-diisopropylethylamine (0.128 mL, 0.732 mmol) and dichloromethane (2 mL) was stirred in a microwave reactor at 130° C. for 1 h, then cooled. 1 M aqueous hydrochloric acid (10 mL) was added and the mixture extracted with dichloromethane. The extracts were dried (MgSO₄), then evaporated under reduced pressure and the residue chromatographed (silica gel, 1-8% methanol/dichloromethane) to give the intermediate ester. 1 M aqueous sodium hydroxide (2.00 mL, 2.00 mmol) was added dropwise to a stirred solution of the intermediate ester in ethanol (10 mL) and the mixture stirred for 18 h, then acidified with 6M aqueous hydrochloric acid (0.5 mL). Water (70 mL) was added and the mixture stirred for 0.5 h. The precipitate was filtered, washed with water and dried to give the title compound (0.071 g, 44%) as a solid. 1H NMR (400 MHz, DMSO-d₆) δ ppm 0.86-1.00 (m, 2 H) 1.01-1.15 (m, 1 H) 1.48-1.57 (m, 1 H) 1.73-1.85 (m, 4 H) 2.52-2.58 (m, 2 H) 3.41-3.53 (m, 1 H) 4.10 (d, J=5.81 Hz, 2 H) 7.68 (dd, J=9.35, 7.07 Hz, 1 H) 7.74-7.79 (m, 2 H) 9.88 (t, J=5.68 Hz, 1 H) 12.97 (br. s., 1 H) 16.31 (s, 1 H).

### EXAMPLE 36

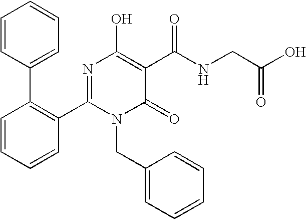

### N-{[2-(2-Biphenylyl)-4-hydroxy-6-oxo-1-(phenylmethyl)-1,6-dihydro-5-pyrimidinyl]carbonyl}glycine

A mixture of N-{[2-(2-bromophenyl)-4-hydroxy-6-oxo-1-(phenylmethyl)-1,6-dihydro-5-pyrimidinyl]carbonyl}glycine (0.185 g, 0.368 mmol), phenylboronic acid (0.090 g, 0.737 mmol), tetrakis(triphenylphosphine)palladium (0) (0.021 g, 0.018 mmol), 2 M aqueous sodium carbonate (0.5 mL, 1.00 mmol) and dioxane (4 mL) was stirred in a microwave reactor at 160° C. for 0.5 h, then cooled. 1 M aqueous sodium hydroxide (2 mL) and water (10 mL) were added and the mixture filtered through a nylon micropore filter. The filtrate was acidified to pH 1 with 6M aqueous hydrochloric acid and extracted with ethyl acetate. The extracts were washed with water, brine, dried (MgSO₄) and evaporated under reduced pressure. The residue was purified by HPLC (ODS, 10-90% acetonitrile/water+0.1% trifluoroacetic acid) to give the title compound (0.143 g, 85%) as a foam. 1H NMR (400 MHz, DMSO-d₆) δ ppm 4.05 (d, J=6.06 Hz, 2 H) 4.47 (d, J=15.41 Hz, 1 H) 4.87 (d, J=15.66 Hz, 1 H) 6.79-6.87 (m, 2 H) 7.15-7.22 (m, 3 H) 7.25-7.31 (m, 2 H) 7.34-7.49 (m, 5 H) 7.56 (d, J=7.58 Hz, 1 H) 7.62-7.70 (m, 1 H) 9.69 (t, J=5.56 Hz, 1 H) 12.93 (br. s., 1 H) 16.09 (s, 1 H).

### EXAMPLE 37

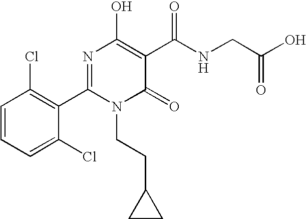

### N-{[1-(2-Cyclopropylethyl)-2-(2,6-dichlorophenyl)-4-hydroxy-6-oxo-1,6-dihydro-5-pyrimidinyl]carbonyl}glycine

37a) 3-(2-Cyclopropylethyl)-2-(2,6-dichlorophenyl)-6-hydroxy-4(3H)-pyrimidinone. A 2 M solution of trimethylaluminium in hexane (1.38 mL, 2.76 mmol) was added to a stirred mixture of 2-(cyclopropyl)ethylamine hydrochloride (PCT Int. Appl. (2004), WO 2004052312, 0.305 g, 2.49 mmol), 2,6-dichlorobenzonitrile (0.516 g, 3.00 mmol) and toluene (5 mL) at room temperature. The mixture was stirred in a microwave reactor at 160° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL) containing 1 M aqueous sodium hydroxide (2 mL), washed with ether, acidified to pH 2 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.478 g, 59%) as a white solid. 1H NMR (400 MHz, DMSO-d₆) δ ppm −0.29-−0.20 (m, 2 H) 0.26-0.34 (m, 2 H) 0.44-0.60 (m, 1 H) 1.33-1.44 (m, 2 H) 3.60-3.72 (m, 2 H) 5.49 (s, 1 H) 7.66 (dd, J=9.09, 7.07 Hz, 1 H) 7.70-7.78 (m, 2 H) 11.80 (br. s., 1 H).

37b) N-{[1-(2-Cyclopropylethyl)-2-(2,6-dichlorophenyl)-4-hydroxy-6-oxo-1,6-dihydro-5-pyrimidinyl]carbonyl}glycine. A mixture of 3-(2-cyclopropylethyl)-2-(2,6-dichlorophenyl)-6-hydroxy-4(3H)-pyrimidinone (0.476, 1.46 mmol), ethyl 2-isocyanatoacetate (0.329 mL, 2.93 mmol), N,N-diisopropylethylamine (0.510 mL, 2.93 mmol) and dichloromethane (5 mL) was stirred in a microwave reactor at 130° C. for 1 h, then cooled. 1 M aqueous hydrochloric acid (10 mL) was added and the mixture extracted with dichloromethane. The extracts were dried (MgSO₄), evaporated under reduced pressure and the residue chromatographed (silica gel, 1-8% methanol/dichloromethane) to give the intermediate ester. 1 M aqueous sodium hydroxide (6.00 mL, 6.00 mmol) was added dropwise to a stirred solution of the intermediate ester in ethanol (25 mL) and the mixture stirred for 18 h, then acidified with 6M aqueous hydrochloric acid. Water (100 mL) was added and the mixture stirred for 0.5 h. The precipitate was filtered, washed with water and dried to give the title compound (0.423 g, 68%) as a white solid. 1H NMR (400 MHz, DMSO-d₆) δ ppm −0.24-−0.14 (m, 2 H) 0.27-0.37 (m, 2 H) 0.46-0.64 (m, 1 H) 1.39-1.50 (m, 2 H) 3.72-3.83 (m, 2 H) 4.12 (d, J=5.56 Hz, 2 H) 7.71 (dd, J=9.35, 6.82 Hz, 1 H) 7.75-7.81 (m, 2 H) 9.85 (t, J=5.68 Hz, 1 H) 12.99 (br. s., 1 H) 16.25 (br. s., 1 H).

### EXAMPLE 38

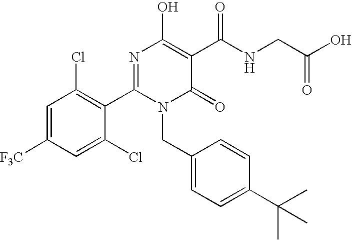

### N-[(2-[2,6-Dichloro-4-(trifluoromethyl)phenyl]-1-{[4-(1,1-dimethylethyl)phenyl]methyl}-4-hydroxy-6-oxo-1,6-dihydro-5-pyrimidinyl)carbonyl]glycine

38a) 2-[2,6-Dichloro-4-(trifluoromethyl)phenyl]-3-{[4-(1,1-dimethylethyl)phenyl]methyl}-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,6-dichloro-4-(trifluoromethyl)benzonitrile (1.20 g, 5.00 mmol) and toluene (15 mL) at room temperature, then the mixture refluxed for 7 h under nitrogen. After cooling, the solvent was removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL) containing 1 M aqueous sodium hydroxide (2 mL), washed with ether, acidified to pH 1 with 6 M aqueous hydrochloric acid, and extracted with ethyl acetate. The extracts were washed with water, brine, dried (MgSO₄) and evaporated under reduced pressure. The residue was chromatographed (silica gel, 1-8% methanol/dichloromethane) and the product triturated with ether. The solid was collected, washed with ether and dried to give the title compound (0.234 g, 20%) as a white solid. 1H NMR (400 MHz, DMSO-d₆) δ ppm 1.23 (s, 9 H) 4.90 (s, 2 H) 5.61 (s, 1 H) 6.77-6.83 (m, 2 H) 7.15-7.22 (m, 2 H) 8.07 (s, 2 H) 12.01 (br. s., 1 H).

38b) N-[(2-[2,6-Dichloro-4-(trifluoromethyl)phenyl-1-{[4-(1,1-dimethylethyl)phenyl]methyl}-4-hydroxy-6-oxo-1,6-dihydro-5-pyrimidinyl)carbonyl]glycine disodium salt. A mixture of 2-[2,6-dichloro-4-(trifluoromethyl)phenyl]-3-{[4-(1,1-dimethylethyl)phenyl]methyl}-6-hydroxy-4(3H)-pyrimidinone (0.232, 0.492 mmol), ethyl 2-isocyanatoacetate (0.111 mL, 0.984 mmol), N,N-diisopropylethylamine (0.171 mL, 0.984 mmol) and dichloromethane (2 mL) was stirred in a microwave reactor at 130° C. for 1 h, then cooled. 1 M Aqueous hydrochloric acid (10 mL) was added and the mixture extracted with dichloromethane. The extracts were dried (MgSO₄), evaporated under reduced pressure and the residue chromatographed (silica gel, 0.5-5% methanol/dichloromethane) to give the intermediate ester. 1 M aqueous sodium hydroxide (3.00 mL, 3.00 mmol) was added dropwise to a stirred solution of the intermediate ester in ethanol (15 mL) and the mixture stirred for 2 h. The precipitate was filtered, washed with ethanol and dried to give the title compound (0.126 g, 42%) as a white solid. 1H NMR (400 MHz, DMSO-d₆) δ ppm 1.22 (s, 9 H) 3.49 (d, J=4.29 Hz, 2 H) 4.75 (s, 2 H) 6.77-6.83 (m, 2 H) 7.11-7.20 (m, 2 H) 7.96 (s, 2 H) 10.10 (t, J=4.17 Hz, 1 H).

### EXAMPLE 39

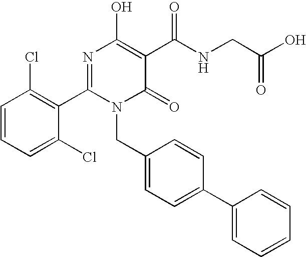

### N-{[1-(4-Biphenylylmethyl)-2-(2,6-dichlorophenyl)-4-hydroxy-6-oxo-1,6-dihydro-5-pyrimidinyl]carbonyl}glycine

39a) 3-(4-Biphenylylmethyl)-2-(2,6-dichlorophenyl)-6-hydroxy-4(3H)-pyrimidinone. A 1 solution of dimethylaluminium chloride in hexane (2.75 mL, 2.75 mmol) was added to a stirred mixture of 4-phenylbenzylamine (0.458 g, 2.50 mmol), 2,6-dichlorobenzonitrile (0.516 g, 3.00 mmol) and toluene (5 mL) at room temperature. The mixture was stirred in a microwave reactor at 160° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with dichloromethane and the solid collected, washed with dichloromethane and dried to give the title compound (0.756 g, 71%) as a solid. 1H NMR (400 MHz, DMSO-d₆) δ ppm 4.95 (s, 2 H) 5.63 (s, 1 H) 6.84-7.01 (m, 2 H) 7.32-7.39 (m, 1 H) 7.42-7.48 (m, 2 H) 7.48-7.54 (m, 2 H) 7.58-7.67 (m, 5H) 11.96 (br. s., 1 H).

39b) N-{[1-(4-Biphenylylmethyl)-2-(2,6-dichlorophenyl)-4-hydroxy-6-oxo-1,6-dihydro-5-pyrimidinyl]carbonyl}glycine disodium salt. A mixture of 3-(4-biphenylylmethyl)-2-(2,6-dichlorophenyl)-6-hydroxy-4(3H)-pyrimidinone (0.753, 1.78 mmol), ethyl 2-isocyanatoacetate (0.399 mL, 3.56 mmol), N,N-diisopropylethylamine (0.619 mL, 3.56 mmol) and dichloromethane (6 mL) was stirred in a microwave reactor at 130° C. for 1 h, then cooled, washed with 1 M aqueous hydrochloric acid (10 mL) and dried (MgSO₄). The solvent was evaporated under reduced pressure and the residue chromatographed (silica gel, 1-9% methanol/dichloromethane) to give the intermediate ester. 1 M Aqueous sodium hydroxide (6.00 mL, 6.00 mmol) was added dropwise to a stirred suspension of the intermediate ester in ethanol (25 mL) and the mixture stirred for 1.25 h. The precipitate was filtered, washed with ethanol and dried to give the title compound (0.236 g, 23%) as a white solid. 1H NMR (400 MHz, DMSO-d₆) δ ppm 3.51 (d, J=4.29 Hz, 2 H) 4.82 (s, 2 H) 6.94-7.01 (m, 2 H) 7.31-7.37 (m, 1 H) 7.40-7.48 (m, 4 H) 7.49-7.53 (m, 3 H) 7.58-7.64 (m, 2 H) 10.18 (t, J=4.17 Hz, 1 H).

### EXAMPLE 40

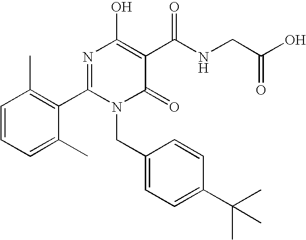

### N-{[1-{[4-(1,1-Dimethylethyl)phenyl]methyl}-2-(2,6-dimethylphenyl)-4-hydroxy-6-oxo-1,6-dihydro-5-pyrimidinyl]carbonyl}glycine

40a) 3-{[4-(1,1-Dimethylethylphenyl]methyl}-2-(2,6-dimethylphenyl)-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,6-dimethylbenzonitrile (0.394 g, 3.00 mmol) and toluene (5 mL) at room temperature. The mixture was stirred in a microwave reactor at 160° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.1 mmol) were added and the mixture refluxed under nitrogen for 4 h. After cooling, the mixture was poured into water (200 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.306 g, 34%) as a white solid. NMR (400 MHz, DMSO-d₆) δ ppm 1.23 (s, 9 H) 1.73 (s, 6 H) 4.79 (s, 2 H) 5.53 (s, 1 H) 6.64-6.70 (m, 2 H) 7.09 (d, J=7.58 Hz, 2 H) 7.16-7.23 (m, 2 H) 7.32 (t, J=7.71 Hz, 1 H) 11.70 (br s, 1 H).

40b) N-{[1-{[4-(1,1-Dimethylethyl)phenyl]methyl}-2-(2,6-dimethylphenyl)-4-hydroxy-6-oxo-1,6-dihydro-5-pyrimidinyl]carbonyl}glycine. A mixture of3-{[4-(1,1-dimethylethyl)phenyl]methyl}-2-(2,6-dimethylphenyl)-6-hydroxy-4(3H)-pyrimidinone (0.296 g, 0.817 mmol), ethyl 2-isocyanatoacetate (0.183 mL, 1.63 mmol), N,N-diisopropylethylamine (0.284 mL, 1.63 mmol) and dichloromethane (3 mL) was stirred in a microwave reactor at 130° C. for 1 h, then cooled, washed with 1 M aqueous hydrochloric acid (10 mL) and dried (MgSO₄). The solvent was evaporated under reduced pressure and the residue chromatographed (silica gel, 1-7% methanol/dichloromethane) to give the intermediate ester. 1 M aqueous sodium hydroxide (3.00 mL, 3.00 mmol) was added dropwise to a stirred suspension of the intermediate ester in ethanol (15 mL) and the mixture stirred for 48 h. 6 M aqueous hydrochloric acid (1 mL) was added, followed by water. A gummy precipitate separated and was redissolved by adjusting the pH to 14 with 6 M aqueous sodium hydroxide. The solution was re-acidified to pH 1 with 6 M aqueous hydrochloric acid while stirring vigorously. The precipitate was filtered, washed with water and dried to give the title compound (0.236 g, 62%) as a solid. 1H NMR (400 MHz, DMSO-d₆) δ ppm 1.23 (s, 9 H) 1.80 (s, 6 H) 4.13 (d, J=5.56 Hz, 2 H) 4.89 (s, 2 H) 6.68-6.76 (m, 2 H) 7.13 (d, J=7.83 Hz, 2 H) 7.18-7.26 (m, 2 H) 7.36 (t, J=7.71 Hz, 1 H) 9.95 (t, J=5.56 Hz, 1 H) 13.00 (br, s., 1 H) 16.17 (br, s., 1 H).

### EXAMPLE 41

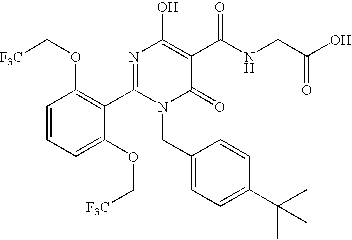

### N-[(2-{2,6-Bis[(2,2,2-trifluoroethyl)oxy]phenyl}-1-{[4-(1,1-dimethylethyl)phenyl]methyl}-4-hydroxy-6-oxo-1,6-dihydro-5-pyrimidinyl)carbonyl]glycine

41a) 2-{2,6-Bis[(2,2,2-trifluoroethyl)oxy]phenyl}-3-{[4-(1,1-dimethylethyl)phenyl]methyl}-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,6-bis[(2,2,2-trifluoroethyl)oxy]benzonitrile (0.898 g, 3.00 mmol) and toluene (5 mL) at room temperature. The mixture was stirred in a microwave reactor at 160° C. for 0.5 h, then cooled and the solvent removed under reduced pressure. Diethyl malonate (1.52 mL, 10.0 mmol), 2-methoxyethanol (15 mL) and 4.37 M sodium methoxide in methanol (2.30 mL, 10.1 mmol) were added and the mixture refluxed under nitrogen for 18 h. After cooling, the mixture was poured into water (200 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.885 g, 67%) as a solid. 1H NMR (400 MHz, DMSO-d₆) δ ppm 1.21 (s, 9 H) 4.15-4.30 (m, 2 H) 4.65-4.78 (m, 2 H) 4.80 (s, 2 H) 5.47 (s, 1 H) 6.70 (d, J=8.34 Hz, 2 H) 6.93 (d, J=8.34 Hz, 2 H) 7.17 (d, J=8.34 Hz, 2 H) 7.54 (t, J=8.46 Hz, 1 H) 11.56 (s, 1 H).

41b) N-[(2-{2,6-Bis[(2,2,2-trifluoroethyl)oxy]phenyl}-1-{[4-(1,1-dimethylethyl)phenyl]methyl}-4-hydroxy-6-oxo-1,6-dihydro-5-pyrimidinyl)carbonyl]glycine. A mixture of 2-{2,6-bis[(2,2,2-trifluoroethyl)oxy]phenyl}-3-{[4-(1,1-dimethylethyl)phenyl]methyl}-6-hydroxy-4(3H)-pyrimidinone (0.882 g, 1.66 mmol), ethyl 2-isocyanatoacetate (0.374 mL, 3.33 mmol), N,N-diisopropylethylamine (0.580 mL, 3.33 mmol) and dichloromethane (6 mL) was stirred in a microwave reactor at 130° C. for 1 h, then cooled and poured into 1 M aqueous hydrochloric acid (10 mL). The mixture was extracted with dichloromethane and the extracts dried (MgSO₄). The solvent was evaporated under reduced pressure and the residue chromatographed (silica gel, 1-7% methanol/dichloromethane) to give the intermediate ester. 1 M aqueous sodium hydroxide (10.0 mL, 10.0 mmol) was added dropwise to a stirred solution of the intermediate ester in ethanol (50 mL) and the mixture stirred for 18 h. 6 M aqueous hydrochloric acid was added to adjust to pH 1 and the mixture diluted with water (100 mL), then extracted with ethyl acetate. The extracts were washed with water and brine, dried (MgSO₄) and evaporated under reduced pressure. The residue was purified by HPLC (ODS, 10-90% acetonitrile/water+0.1% trifluoroacetic acid) and the product triturated with aqueous acetonitrile. The solid was collected, washed with water and dried to give the title compound (0.159 g, 15%) as a cream powder. 1H NMR (400 MHz, DMSO-d₆) δ ppm 1.22 (s, 9 H) 4.11 (d, J=5.56 Hz, 2 H) 4.32-4.47 (m, 2 H) 4.74-4.86 (m, 2 H) 4.92 (s, 2 H) 6.78 (d, J=8.34 Hz, 2 H) 6.98 (d, J=8.59 Hz, 2 H) 7.21 (d, J=8.34 Hz, 2 H) 7.60 (t, J=8.59 Hz, 1 H) 9.87 (t, J=5.56 Hz, 1 H) 12.95 (s, 1 H) 16.08 (s, 1 H).

### EXAMPLE 42

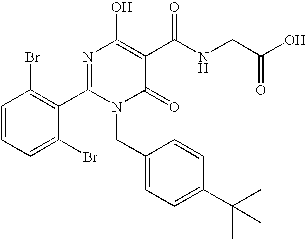

### N-[(2-(2,6-Dibromolphenyl)-1-{[4-(1,1-dimethylethyl)phenyl]methyl}-4-hydroxy-6-oxo-1,6-dihydro-5-pyrimidinyl)carbonyl]glycine

42a) 2,6-Dibromobenzonitrile. 2,6-Dibromoaniline (0.251 g, 1.00 mmol) was added to a stirred solution of copper (I) cyanide (0.116 g, 1.30 mmol) in dimethylsulfoxide (10 mL) at 50° C. under nitrogen. tert-Butyl nitrite (0.357 mL, 3.00 mmol) was injected over 5 min and the mixture stirred for 1 h at 50° C., then cooled and poured into 1 M aqueous hydrochloric acid (100 mL). The mixture was extracted with ethyl acetate and the extracts washed with water, brine, then dried (MgSO₄). The solvent was evaporated under reduced pressure and the residue chromatographed (silica gel, 1-9% methanol/dichloromethane) to give the title compound (0.100 g, 38%). 1H NMR (400 MHz, CHLOROFORM-d) δ ppm 7.33 (t, J=8.08 Hz, 1 H) 7.67 (d, J=8.08 Hz, 2 H).

42b) 2-(2,6-Dibromophenyl)-3-{[4-(1,1-dimethylethyl)phenyl]methyl}-6-hydroxy-4(3H)-pyrimidinone. A 1 M solution of dimethylaluminium chloride in hexane (2.20 mL, 2.20 mmol) was added to a stirred mixture of 4-tert-butylbenzylamine (0.326 g, 2.00 mmol), 2,6-dibromobenzonitrile (0.587 g, 2.25 mmol) and toluene (6 mL) at room temperature. The mixture was stirred in a microwave reactor at 160° C. for 0.5 h, then cooled and the solvent removed under reduced pressure. Diethyl malonate (1.28 g, 8.00 mmol), 2-methoxyethanol (12 mL) and 4.37 M sodium methoxide in methanol (1.83 mL, 8.00 mmol) were added and the mixture refluxed under nitrogen for 18 h. After cooling, the mixture was poured into water (150 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.361 g, 37%) as a cream solid. 1H NMR (400 MHz, DMSO-d₆) δ ppm 1.23 (s, 9 H) 4.86 (s, 2 H) 5.59 (s, 1 H) 6.74-6.80 (m, 2 H) 7.17-7.22 (m, 2 H) 7.43 (t, J=8.08 Hz, 1 H) 7.76 (d, J=8.08 Hz, 2 H) 11.86 (s, 1 H).

42c) N-[(2-(2,6-Dibromophenyl)-1-{[4-(1,1-dimethylethyl)phenyl]methyl}-4-hydroxy-6-oxo-1,6-dihydro-5-pyrimidinyl)carbonyl]glycine. A mixture of 2-(2,6-dibromophenyl)-3-{[4-(1,1-dimethylethyl)phenyl]methyl}-6-hydroxy-4(3H)-pyrimidinone (0.360 g, 0.731 mmol), ethyl 2-isocyanatoacetate (0.165 mL, 1.46 mmol), N,N-diisopropylethylamine (0.255 mL, 1.46 mmol) and dichloromethane (4 mL) was stirred in a microwave reactor at 130° C. for 1 h, then cooled, washed with 1 M aqueous hydrochloric acid (10 mL) and dried (MgSO₄). The solvent was evaporated under reduced pressure and the residue chromatographed (silica gel, 1-7% methanol/dichloromethane) to give the intermediate ester. 1 M aqueous sodium hydroxide (3.00 mL, 3.00 mmol) was added dropwise to a stirred solution of the intermediate ester in ethanol (15 mL) and the mixture stirred for 2 h. 6 M aqueous hydrochloric acid (1 mL) was added and the mixture diluted with water (80 mL) and stirred 0.5 h. The precipitate was filtered, washed with water and dried to give the title compound (0.216 g, 50%) as a cream powder. 1H NMR (400 MHz, DMSO-d₆) δ ppm 1.23 (s, 9 H) 4.14 (d, J=5.56 Hz, 2 H) 4.98 (s, 2 H) 6.82-6.87 (m, 2 H) 7.20-7.27 (m, 2 H) 7.48 (t, J=8.08 Hz, 1 H) 7.81 (d, J=8.08 Hz, 2 H) 9.88 (t, J=5.68 Hz, 1 H) 12.98 (br. s., 1 H) 16.36 (s, 1 H).

### EXAMPLE 43

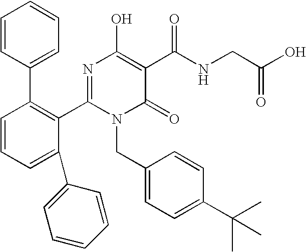

### N-{[1-{[4-(1,1-Dimethylethyl)phenyl]methyl}-4-hydroxy-6-oxo-2-(1,1′:3′,1″-terphenyl-2′-yl)-1,6-dihydro-5-pyrimidinyl]carbonyl}glycine

A mixture of N-[(2-(2,6-dibromophenyl)-1-{[4-(1,1-dimethylethyl)phenyl]methyl}-4-hydroxy-6-oxo-1,6-dihydro-5-pyrimidinyl)carbonyl]glycine (0.144 g, 0.243 mmol), phenylboronic acid (0.118 g, 0.971 mmol), tetrakis(triphenylphosphine)palladium (0) (0.023 g, 0.020 mmol), 2 M aqueous sodium carbonate (1 mL, 2 mmol) and dioxane (4 mL) was stirred in a microwave reactor at 160° C. for 0.5 h, then cooled. 1 M aqueous sodium hydroxide (2 mL) and water (10 mL) were added and the mixture filtered through a nylon micropore filter. The filtrate was diluted with water (30 mL), then acidified to pH 1 with 6M aqueous hydrochloric acid. The precipitate was filtered, washed with water, dried and purified by HPLC (ODS, 10-90% acetonitrile/water+0.1% trifluoroacetic acid). The product was reprecipitated from acetic acid with water, filtered, washed with water and dried to give the title compound (0.085 g, 60%) as a cream solid. 1H NMR (400 MHz, DMSO-d₆) δ ppm 1.21 (s, 9 H) 4.02 (d, J=5.56 Hz, 2 H) 4.60 (s, 2 H) 6.42-6.48 (m, 2 H) 6.97-7.08 (m, 4 H) 7.10-7.16 (m, 2 H) 7.19-7.34 (m, 6 H) 7.51 (d,J=7.83 Hz, 2 H) 7.79 (t, J=7.71 Hz, 1 H) 9.64 (t, J=5.56 Hz, 1 H) 12.92 (br. s., 1 H) 15.93 (s, 1 H).

### EXAMPLE 44

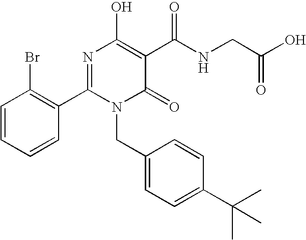

### N-[(2-(2-Bromophenyl)-1-{[4-(1,1-dimethylethyl)phenyl]methyl}-4-hydroxy-6-oxo-1,6-dihydro-5-pyrimidinyl)carbonyl]glycine

44a) 2-(2-Bromophenyl)-3-{[4-(1,1-dimethylethyl)phenyl]methyl}-6-hydroxy-4(3H)-pyrimidinone. A 1 M solution of dimethylaluminium chloride in hexane (5.50 mL, 5.50 mmol) was added to a stirred mixture of 4-tert-butylbenzylamine (0.816 g, 5.00 mmol), 2-bromobenzonitrile (1.09 g, 6.00 mmol) and toluene (5 mL) at room temperature. After stirring for 10 min at room temperature, the mixture was stirred in a microwave reactor at 150° C. for 0.5 h, then cooled and the solvent removed under reduced pressure. Diethyl malonate (3.20 g, 20.0 mmol), 2-methoxyethanol (30 mL) and 4.37 M sodium methoxide in methanol (4.60 mL, 20.1 mmol) were added and the mixture refluxed under nitrogen for 20 h. After cooling, the mixture was poured into water (300 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (1.13 g, 55%) as a cream solid. 1H NMR (400 MHz, DMSO-d₆) δ ppm 1.23 (s, 9 H) 4.55 (d, J=15.41 Hz, 1 H) 5.18 (d, J=15.41 Hz, 1 H) 5.53 (s, 1 H) 6.79-6.84 (m, 2 H) 7.19-7.26 (m, 2 H) 2 H) 7.33-7.38 (m, 1 H) 7.39-7.48 (m, 2 H) 7.75 (dd, J=7.83, 1.26 Hz, 1 H) 11.77 (s, 1 H).

44b) N-[(2-(2-Bromophenyl)-1-{[4-(1,1-dimethylethyl)phenyl]methyl}-4-hydroxy-6-oxo-1,6-dihydro-5-pyrimidinyl)carbonyl]glycine. A mixture of 2-(2-bromophenyl)-3-{[4-(1,1-dimethylethyl)phenyl]methyl}-6-hydroxy-4(3H)-pyrimidinone (1.13 g, 2.73 mmol), ethyl 2-isocyanatoacetate (0.614 mL, 5.47 mmol), N,N-diisopropylethylamine (0.952 mL, 5.47 mmol) and dichloromethane (8 mL) was stirred in a microwave reactor at 130° C. for 1 h, then cooled, washed with 1 M aqueous hydrochloric acid (20 mL) and dried (MgSO₄). The solvent was evaporated under reduced pressure to give the crude intermediate ester. 1 M aqueous sodium hydroxide (20.0 mL, 20.0 mmol) was added dropwise to a stirred, ice-cooled solution of the crude intermediate ester in ethanol (75 mL) and the mixture stirred for 3 h at room temperature. 6 M aqueous hydrochloric acid was added to adjust to pH 1 and the mixture diluted with water (350 mL) and extracted with ethyl acetate. The extracts were washed with water and brine, dried (MgSO₄) and evaporated under reduced pressure to give the crude product (1.50 g) as a yellow foam. 0.155 g of the foam was purified by HPLC (ODS, 10-90% acetonitrile/water+0.1% trifluoroacetic acid) to give the title compound (0.119 g, 82%) as a solid. 1H NMR (400 MHz, DMSO-d₆) δ ppm 1.23 (s, 9 H) 4.11 (d, J=6.32 Hz, 2 H) 4.77 (d, J=15.41 Hz, 1 H) 5.16 (d, J=15.41 Hz, 1 H) 6.87-6.96 (m, 2 H) 7.21-7.30 (m, 2 H) 7.45-7.61 (m, 3 H) 7.71-7.81 (m, 1 H) 9.83 (t, J=5.68 Hz, 1 H) 12.93 (br. s., 1 H) 16.19 (s, 1 H).

### EXAMPLE 45

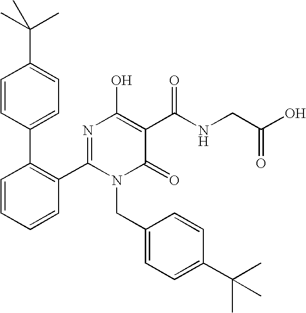

### N-[(2-[4′-(1,1-Dimethylethyl)-2-biphenylyl]-1-{[4-(1,1-dimethylethyl)phenyl]methyl}-4-hydroxy-6-oxo-1,6-dihydro-5-pyrimidinyl)carbonyl]glycine

A mixture of N-[(2-(2-bromophenyl)-1-{[4-(1,1-dimethylethyl)phenyl]methyl}-4-hydroxy-6-oxo-1,6-dihydro-5-pyrimidinyl)carbonyl]glycine (0.130 g, 0.253 mmol), 4-tert-butylphenylboronic acid (0.089 g, 0.500 mmol), tetrakis(triphenylphosphine)palladium (0) (0.023 g, 0.020 mmol), 2 M aqueous sodium carbonate (0.5 mL, 1 mmol) and dioxane (1 mL) was stirred in a microwave reactor at 160° C. for 0.5 h, then cooled. 1 M aqueous hydrochloric acid (3 mL) was added and the mixture extracted with ethyl acetate. The extracts were dried (MgSO₄), filtered through a PTFE (Teflon®) micropore filter and evaporated under reduced pressure. The residue was purified by HPLC (ODS, 10-90% acetonitrile/water+0.1% trifluoroacetic acid) to give the title compound (0.081 g, 56%) as a foam. 1H NMR (400 MHz, DMSO-d₆) δ ppm 1.20 (s, 9 H) 1.28 (s, 9 H) 4.05 (d, J=5.56 Hz, 2 H) 4.57 (d, J=15.16 Hz, 1 H) 4.75 (d, J=15.41 Hz, 1 H) 6.66-6.76(m, 2 H) 7.13-7.19 (m, 4 H) 7.21-7.28 (m, 1 H) 7.37-7.43 (m, 1 H) 7.44-7.49 (m, 1 H) 7.50-7.56 (m, 2 H) 7.62-7.68 (m, 1 H) 9.71 (t, J=5.56 Hz, 1 H) 12.91 (br. s., 1 H) 16.06 (s, 1 H).

### EXAMPLE 46

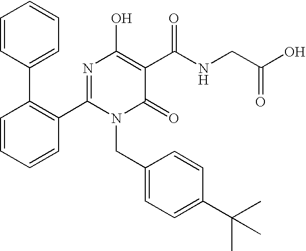

### N-[(2-(2-Biphenylyl)-1-{[4-(1,1-dimethylethyl)phenyl]methyl}-4-hydroxy-6-oxo-1,6-dihydro-5-pyrimidinyl)carbonyl]glycine

A mixture of N-[(2-(2-bromophenyl)-1-{[4-(1,1-dimethylethyl)phenyl]methyl}-4-hydroxy-6-oxo-1,6-dihydro-5-pyrimidinyl)carbonyl]glycine (0.130 g, 0.253 mmol), phenylboronic acid (0.061 g, 0.500 mmol), tetrakis(triphenylphosphine)palladium (0) (0.023 g, 0.020 mmol), 2 M aqueous sodium carbonate (0.5 mL, 1 mmol) and dioxane (1 mL) was stirred in a microwave reactor at 160° C. for 0.5 h, then cooled. 1 M aqueous hydrochloric acid (3 mL) was added and the mixture extracted with ethyl acetate. The extracts were dried (MgSO₄), filtered through a PTFE micropore filter and evaporated under reduced pressure. The residue was purified by HPLC (ODS, 10-90% acetonitrile/water+0.1% trifluoroacetic acid) to give the title compound (0.095 g, 74%) as a foam. 1H NMR (400 MHz, DMSO-d₆) δ ppm 1.20 (s, 9 H) 4.05 (d, J=5.81 Hz, 2 H) 4.52 (d, J=15.41 Hz, 1 H) 4.73 (d, J=15.16 Hz, 1 H) 6.77 (d, J=8.34 Hz, 2 H) 7.15-7.25 (m, 4 H) 7.31-7.43 (m, 3 H) 7.47-7.52 (m, 1 H) 7.53-7.58 (m, 2 H) 7.63-7.71 (m, 1 H) 9.68 (t, J=5.68 Hz, 1 H) 12.91 (br. s., 1 H) 16.05 (s, 1 H).

### EXAMPLE 47

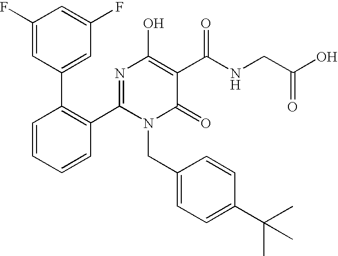

### N-[(2-(3′,5′-Difluoro-2-biphenylyl)-1-{[4-(1,1-dimethylethyl)phenyl]methyl}-4-hydroxy-6-oxo-1,6-dihydro-5-pyrimidinyl)carbonyl]glycine

A mixture of N-[(2-(2-bromophenyl)-1-{[4-(1,1-dimethylethyl)phenyl]methyl}-4-hydroxy-6-oxo-1,6-dihydro-5-pyrimidinyl)carbonyl]glycine (0.130 g, 0.253 mmol), 3,5-difluorophenylboronic acid (0.079 g, 0.500 mmol), tetrakis(triphenylphosphine)palladium (0) (0.023 g, 0.020 mmol), 2 M aqueous sodium carbonate (0.5 mL, 1 mmol) and dioxane (1 mL) was stirred in a microwave reactor at 160° C. for 0.5 h, then cooled. 1 M aqueous hydrochloric acid (3 mL) was added and the mixture extracted with ethyl acetate. The extracts were dried (MgSO₄), filtered through a PTFE micropore filter and evaporated under reduced pressure. The residue was purified by HPLC (ODS, 10-90% acetonitrile/water+0.1% trifluoroacetic acid) to give the title compound (0.084 g, 60%) as a foam. 1H NMR (400 MHz, DMSO-d₆) δ ppm 1.20 (s, 9 H) 4.07 (d, J=5.56 Hz, 2 H) 4.76 (d, J=15.41 Hz, 1 H) 4.88 (d, J=15.16 Hz, 1 H) 6.65-6.76 (m, 4 H) 7.15-7.26 (m, 3 H) 7.52-7.63 (m, 2 H) 7.64-7.73 (m, 2 H) 9.76 (t, J=5.43 Hz, 1 H) 12.96 (br. s., 1 H) 16.10 (br. s., 1 H).

### EXAMPLE 48

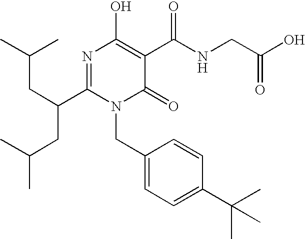

### N-(}1-{[4-(1,1-Dimethylethyl)phenyl]methyl}-4-hydroxy-2-[3-methyl-1-(2-methyl]propyl)butyl]-6-oxo-1,6-dihydro-5-pyrimidinyl}carbonyl)glycine

48a) 4-Methyl-2-(2-methylpropyl)pentanenitrile. Potassium tert-butoxide (2.81 g, 25.0 mmol) was added in one portion to an ice-cooled, stirred mixture of tosylmethyl isocyanide (2.54 g, 13.0 mmol) and dimethylsulfoxide (35 mL) under nitrogen. After 5 min, methanol (0.5 mL) was added, followed by 2,6-dimethyl-4-heptanone (1.76 mL, 10.0 mmol), and the mixture stirred at room temperature for 72 h, then poured into 0.1 M aqueous hydrochloric acid (350 mL) and extracted with hexane. The extracts were washed with brine, dried (MgSO₄) and evaporated under reduced pressure at room temperature. The residue was chromatographed twice (silica gel, 0-40% ethyl acetate/hexane) to give the title compound (0.368 g, 24%) as an oil. 1H NMR (400 MHz, CHLOROFORM-d) δ ppm 0.94-0.99 (m, 12 H) 1.27-1.37 (m, 2 H) 1.54-1.69 (m, 2 H) 1.81-1.96 (m, 2 H) 2.56-2.69 (m, 1 H).

48b) 3-{[4-(1,1-Dimethylethyl)phenyl]methyl}-6-hydroxy-2-[3-methyl-1-(2-methylpropyl)butyl]-4(3H)-pyrimidinone. A 1 M solution of dimethylaluminium chloride in hexane (1.09 mL, 1.09 mmol) was added to a stirred mixture of 4-tert-butylbenzylamine (0.162 g, 0.992 mmol), 4-methyl-2-(2-methylpropyl)pentanenitrile (0.182 g, 1.19 mmol) and toluene (2 mL) at room temperature. After stirring for 10 min at room temperature, the mixture was stirred in a microwave reactor at 150° C. for 0.5 h, then cooled and the solvent removed under reduced pressure. Diethyl malonate (0.640 g, 4.00 mmol), 2-methoxyethanol (6 mL) and 4.37 M sodium methoxide in methanol (0.915 mL, 4.00 mmol) were added and the mixture refluxed under nitrogen for 20 h. After cooling, the mixture was poured into water (100 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was chromatographed (silica gel, 1-9% methanol/dichloromethane) to give the title compound (0.156 g, 41%). 1H NMR (400 MHz, DMSO-d₆) δ ppm 0.51 (br. s., 6 H) 0.69 (br. s., 6 H) 1.16-1.35 (m, 4 H) 1.24 (s, 9 H) 1.43-1.54 (m, 2 H) 2.68-2.78 (pent, J=6.69 Hz, 1 H) 5.20 (br. s., 2 H) 5.39 (s, 1 H) 7.01 (d, J=8.34 Hz, 2 H) 7.37 (d, J=8.34 Hz, 2 H) 11.24 (br. s., 1 H).

48c) N-({1-{]4-(1,1-Dimethylethyl)phenyl]methyl}-4-hydroxy-2-[3-methyl-1-(2-methylpropyl)butyl]-6-oxo-1,6-dihydro-5-pyrimidinyl}carbonyl)glycine. A mixture of 3-{[4-(1,1-dimethylethyl)phenyl]methyl}-6-hydroxy-2-[3-methyl-1-(2-methylpropyl)butyl]-4(3H)-pyrimidinone (0.154 g, 0.398 mmol), ethyl 2-isocyanatoacetate (0.089 mL, 0.796 mmol), N,N-diisopropylethylamine (0.139 mL, 0.796 mmol) and dichloromethane (1 mL) was stirred in a microwave reactor at 130° C. for 1 h, then cooled. 1 M aqueous hydrochloric acid (3 mL) was added and the mixture extracted with dichloromethane. The extracts were dried (MgSO₄), evaporated under reduced pressure and the residue chromatographed (silica gel, 1-6% methanol/dichloromethane) to give the crude intermediate ester. 1 M aqueous sodium hydroxide (1.50 mL, 1.50 mmol) was added dropwise to a stirred solution of the crude intermediate ester in ethanol (7 mL) and the mixture stirred for 4 h at room temperature. 6 M aqueous hydrochloric acid (1 mL) was added and the mixture diluted with water (70 mL) and extracted with ethyl acetate. The extracts were washed with water and brine, dried (MgSO₄) and evaporated under reduced pressure to give the title compound (0.099 g, 51%) as a foam. 1H NMR (400 MHz, DMSO-d₆) δ ppm 0.52 (br. s., 6 H) 0.70 (br. s., 6 H) 1.20-1.38 (m, 4 H) 1.24 (s, 9 H) 1.46-1.58 (m, 2 H) 2.80 (pent, J=6.57 Hz, 1 H) 4.10 (d, J=5.81 Hz, 2 H) 5.29 (br. s., 2 H) 7.06-7.11 (m, 2 H) 7.35-7.42 (m, 2 H) 9.88 (t, J=5.68 Hz, 1 H) 12.95 (br. s., 1 H) 15.91 (s, 1 H).

### EXAMPLE 49

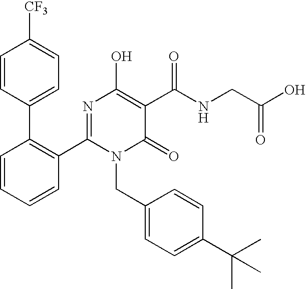

### N-({1-{[4-(1,1-Dimethylethyl)phenyl]methyl}-4-hydroxy-6-oxo-2-[4′-(trifluoromethyl)-2-biphenylyl]-1,6-dihydro-5-pyrimidinyl}carbonyl)glycine

A mixture of N-[(2-(2-bromophenyl)-1-{[4-(1,1-dimethylethyl)phenyl]methyl}-4-hydroxy-6-oxo-1,6-dihydro-5-pyrimidinyl)carbonyl]glycine (0.130 g, 0.253 mmol), 4-(trifluoromethyl)phenylboronic acid (0.95 g, 0.500 mmol), tetrakis(triphenylphosphine)palladium (0) (0.023 g, 0.020 mmol), 2 M aqueous sodium carbonate (0.5 mL, 1 mmol) and dioxane (1 mL) was stirred in a microwave reactor at 160° C. for 0.5 h, then cooled. 1 M aqueous hydrochloric acid (3 mL) was added and the mixture extracted with ethyl acetate. The extracts were dried (MgSO₄), filtered through a PTFE micropore filter and evaporated under reduced pressure. The residue was purified by HPLC (ODS, 10-90% acetonitrile/water+0.1% trifluoroacetic acid) to give the title compound (0.066 g, 45%) as a foam. 1H NMR (400 MHz, DMSO-d₆) δ ppm 1.20 (s, 9 H) 4.06 (d, J=5.56 Hz, 2 H) 4.73 (s, 2 H) 6.71 (d, J=8.34 Hz, 2 H) 7.13-7.20 (m, 2 H) 7.33 (d, J=8.08 Hz, 2 H) 7.55-7.61 (m, 2 H) 7.62-7.66 (m, 1 H) 7.67-7.75 (m, 3 H) 9.72 (t, J=5.68 Hz, 1 H) 12.92 (s, 1 H) 16.08 (s, 1 H).

### EXAMPLE 50

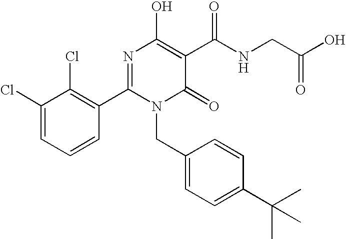

### N-[(2-(2,3-Dichlorophenyl)-1-{[4-(1,1-dimethylethyl)phenyl]methyl}-4-hydroxy-6-oxo-1,6-dihydro-5-pyrimidinyl)carbonyl]glycine

50a) 2-(2,3-Dichlorophenyl)-3-{[4-(1,1-dimethylethyl)phenyl]methyl}-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,3-dichlorobenzonitrile (0.516 g, 3.00 mmol) and toluene (5 mL) at room temperature. The mixture was stirred in a microwave reactor at 150° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.0 mmol) were added and the mixture refluxed under nitrogen for 18 h. After cooling, the mixture was poured into water (200 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.649 g, 64%) as a solid. 1H NMR (400 MHz, DMSO-d₆) δ ppm 1.23 (s, 9 H) 4.86 (d, J=15.41 Hz, 1 H) 4.92 (d, J=15.41 Hz, 1 H) 5.56 (s, 1 H) 6.73-6.77 (m, 2 H) 7.17-7.23 (m, 2 H) 7.44 (t, J=7.71 Hz, 1 H) 7.47 (dd, J=7.83, 2.02 Hz, 1 H) 7.79 (dd, J=7.58, 2.02 Hz, 1 H) 11.79 (br. s., 1 H).

50b) N-[(2-(2,3-Dichlorophenyl)-1-{[4-(1,1-dimethylethyl)phenyl]methyl}-4-hydroxy-6-oxo-1,6-dihydro-5-pyrimidinyl)carbonyl]glycine, disodium salt. A mixture of 2-(2,3-dichlorophenyl)-3-{[4-(1,1-dimethylethyl)phenyl]methyl}-6-hydroxy-4(3H)-pyrimidinone (0.646 g, 1.60 mmol), ethyl 2-isocyanatoacetate (0.360 mL, 3.21 mmol), N,N-diisopropylethylamine (0.559 mL, 3.21 mmol) and dichloromethane (5 mL) was stirred in a microwave reactor at 130° C. for 1 h, then cooled. 1 M aqueous hydrochloric acid (5 mL) was added and the mixture extracted with dichloromethane. The extracts were dried (MgSO₄), and evaporated under reduced pressure to give the crude intermediate ester. 1 M aqueous sodium hydroxide (10.0 mL, 10.0 mmol) was added dropwise to a stirred solution of the crude intermediate ester in ethanol (40 mL) and the mixture stirred for 3 h at room temperature. The precipitate was filtered, washed with 5% aqueous ethanol and dried to give the title compound (0.526 g, 60%) as a cream solid. 1H NMR (400 MHz, DMSO-d₆) δ ppm 1.23 (s, 9 H) 3.49 (d, J=4.29 Hz, 2 H) 4.42 (d, J=15.66 Hz, 1 H) 5.09 (d, J=15.66 Hz, 1 H) 6.75-6.81 (m, 2 H) 7.16 (dd, J=7.71, 1.39 Hz, 1 H) 7.18-7.21 (m, 2 H) 7.32 (t, J=7.96 Hz, 1 H) 7.69 (dd, J=8.08, 1.52 Hz, 1 H) 10.14 (t, J=4.17 Hz, 1 H).

### EXAMPLE 51

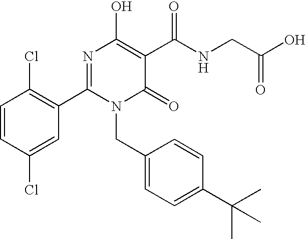

### N-[(2-(2,5-Dichlorophenyl)-1-{[4-(1,1-dimethylethyl)phenyl]methyl}-4-hydroxy-6-oxo-1,6-dihydro-5-pyrimidinyl)carbonyl]glycine

51a) 2-(2,5-Dichlorophenyl)-3-{[4-(1,1-dimethylethyl)phenyl]methyl}-6-hydroxy-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,5-dichlorobenzonitrile (0.516 g, 3.00 mmol) and toluene (3 mL) at room temperature. The mixture was stirred in a microwave reactor at 150° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.0 mmol) were added and the mixture refluxed under nitrogen for 18 h. After cooling, the mixture was poured into water (200 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was chromatographed (silica gel, 1-10% methanol/dichloromethane) to give the title compound (0.529 g, 53%) as a yellow foam. 1H NMR (400 MHz, DMSO-d₆) δ ppm 1.24 (s, 9 H) 4.57 (d, J=15.66 Hz, 1 H) 5.23 (d, J=15.66 Hz, 1 H) 5.56 (s, 1 H) 6.74-6.81 (m, 2 H) 7.21-7.25 (m, 2 H) 7.34 (t, J=1.39 Hz, 1 H) 7.61 (d, J=1.52 Hz, 2 H) 11.81 (br. s., 1 H).

51b) N-[(2-(2,5-Dichlorophenyl)-1-{[4-(1,1-dimethylethyl)phenyl]methyl}-4-hydroxy-6-oxo-1,6-dihydro-5-pyrimidinyl)carbonyl]glycine. A mixture of 2-(2,5-dichlorophenyl)-3-{[4-(1,1-dimethylethyl)phenyl]methyl}-6-hydroxy-4(3H)-pyrimidinone (0.526 g, 1.30 mmol), ethyl 2-isocyanatoacetate (0.293 mL, 2.61 mmol), N,N-diisopropylethylamine (0.455 mL, 2.61 mmol) and dichloromethane (4 mL) was stirred in a microwave reactor at 130° C. for 1 h, then cooled. 1 M aqueous hydrochloric acid (5 mL) was added and the mixture extracted with dichloromethane. The extracts were dried (MgSO₄), evaporated under reduced pressure and the residue chromatographed (silica gel, 1-6% methanol/dichloromethane) to give the intermediate ester. 1 M aqueous sodium hydroxide (5.00 mL, 5.00 mmol) was added dropwise to a stirred solution of the intermediate ester in ethanol (20 mL) and the mixture stirred for 2 h at room temperature. 6 M aqueous hydrochloric acid (2 mL) was added and most of the ethanol removed under reduced pressure. The mixture was diluted with water (80 mL) and extracted with ethyl acetate. The extracts were washed with water, brine, dried (MgSO₄) and evaporated under reduced pressure. The residue was purified by HPLC (ODS, 10-90% acetonitrile/water+0.1% trifluoroacetic acid) to give the title compound (0.202 g, 31%) as a foam. 1H NMR (400 MHz, DMSO-d₆) δ ppm 1.24 (s, 9 H) 4.12 (d, J=5.81 Hz, 2 H) 4.80 (d, J=15.66 Hz, 1 H) 5.18 (d, J=15.66 Hz, 1 H) 6.84-6.93 (m, 2 H) 7.21-7.29 (m, 2 H) 7.59 (d, J=2.02 Hz, 1 H) 7.63 (d, J=8.08 Hz, 1 H) 7.66 (dd, J=8.84, 2.27 Hz, 1 H) 9.84 (t, J=5.68 Hz, 1 H) 12.94 (br s, 1 H) 16.25 (s, 1 H).

### EXAMPLE 52

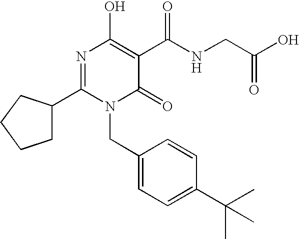

### N-[(2-Cyclopentyl-1-{[4-(1,1-dimethylethyl)phenyl]methyl}-4-hydroxy-6-oxo-1,6-dihydro-5-pyrimidinyl)carbonyl]glycine

52a) 2-Cyclopentyl-3-{[4-(1,1-dimethylethyl)phenyl]methyl}-6-hydroxy-4(3H)-pyrimidinone. Dimethylaluminium chloride (1.212 ml, 1.212 mmol) was added to a solution of 2-cyclopentanecarbonitrile (0.138 ml, 1.322 mmol) and 4-t-Butylbenzylamine (0.194 ml, 1.102 mmol) in Toluene (1.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in Methoxyethanol (4.0 ml). Diethylmalonate (0.668 ml, 4.41 mmol) and sodium methoxide (1.008 ml, 4.41 mmol) were added and the mixture was stirred at reflux for 15 h. After cooling, the mixture was poured into water. The pH was adjusted to ca. 5 by te addition of 1 N HCl. The resulting precipitate was collected, washed with water, dried and purified on silica gel (0-9% MeOH in chloroform) to afford 2-cyclopentyl-3-{[4-(1,1-dimethylethyl)phenyl]methyl}-6-hydroxy-4(3H)-pyrimidinone (287 mg, 0.671 mmol, 80.1% yield). This product was used in the next step as is. LCMS (ES+) m/z 327 (MH⁺).

52b) N-[(2-Cyclopentyl-1-{[4-(1,1-dimethylethyl)phenyl]methyl}-4-hydroxy-6-oxo-1,6-dihydro-5-pyrimidinyl)carbonyl]glycine. A solution of 2-cyclopentyl-3-{[4-(1,1-dimethylethyl)phenyl]methyl}-6-hydroxy-4(3H)-pyrimidinone (280 mg, 0.86 mmol), Hunig's base (0.300 mL, 1.72 mmol) and ethyl isocyanatoacetate (0.193 mL, 1.72 mmol) in Dichloromethane (DCM) (3.0 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (5 mL) and 1 M NaOH (5 mL, 5.00 mmol) and stirred at rt for 3 h. It was then puored into water and acidified by the addition of 6 N HCl. The precipitate was collected, washed with water and purified by RP-HPLC (10 to 95% Acetonitrile in water, plus 0.1% TFA) to afford the title compound (48 mg, 0.112 mmol, 13.06% yield); white powder. ¹H-NMR (400 MHz, CHLOROFORM-d) δ ppm 9.96 (t, J=5.31 Hz, 1 H), 7.37 (d, J=8.34 Hz, 2 H), 7.05 (d, J=8.34 Hz, 2 H), 5.33 (br. s., 2 H), 4.23 (d, J=5.56 Hz, 2 H), 3.15 (quin, J=7.83 Hz, 1 H), 1.93-2.05 (m, 2 H), 1.78-1.92 (m, 4 H), 1.50-1.66 (m, 1 H), 1.30 (s, 9 H), 0.89 (t, J=6.82 Hz, 1 H). LCMS (ES+) m/z 428 (MH⁻).

### EXAMPLE 53

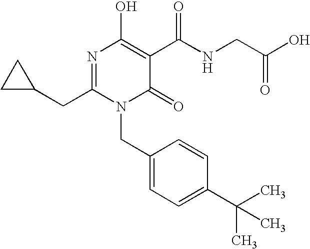

### N-[(2-(Cyclopropylmethyl)-1-{[4-(1,1-dimethylethyl)phenyl]methyl}-4-hydroxy-6-oxo-1,6-dihydro-5-pyrimidinyl)carbonyl]glycine

53a) 2-(Cyclopropylmethyl)-3-{[4-(1,1-dimethylethyl)phenyl]methyl}-6-hydroxy-4(3H)-pyrimidinone. Dimethylaluminium chloride (1.212 ml, 1.212 mmol) was added to a solution of cyclopropylacetonitrile (0.131 ml, 1.322 mmol) and 4-t-Butylbenzylamine (0.194 ml, 1.102 mmol) in Toluene (1.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in Methoxyethanol (4.0 ml). Diethylmalonate (0.668 ml, 4.41 mmol) and sodium methoxyde (1.008 ml, 4.41 mmol) were added and the mixture was stirred at reflux for 9 h. After cooling, the mixture was poured into water. The pH was adjusted to ca. 7 by te addition of 6 N HCl. The resulting precipitate was collected, washed with water, dried and purified on silica gel (0-9% MeOH in chloroform) to afford 2-cyclopentyl-3-{[4-(1,1-dimethylethyl)phenyl]methyl}-6-hydroxy-4(3H)-pyrimidinone (268 mg, 0.857 mmol, 77.84% yield); yellow oil, solidify on standing. This product was used in the next step as is. LCMS (ES⁺) m/z 313 (MH⁺).

53b) N-[(2-(Cyclopropylmethyl)-1-{[4-(1,1-dimethylethyl)phenyl]methyl}-4-hydroxy-6-oxo-1,6-dihydro-5-pyrimidinyl)carbonyl]glycine. A solution of 2-(cyclopropylmethyl)-3-{[4-(1,1-dimethylethyl)phenyl]methyl}-6-hydroxy-4(3H)-pyrimidinone (265 mg, 0.85 mmol), Hunig's base (0.296 mL, 1.70 mmol) and ethyl isocyanatoacetate (0.191 mL, 1.70 mmol) in Dichloromethane (DCM) (3.0 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (5 mL) and 1 M NaOH (5 mL, 5.00 mmol) and stirred at rt for 3.5 h. It was then puored into water and acidified to pH-5 by the addition of 6 N HCl. The precipitate was collected, washed with water and purified by RP-HPLC (10 to 95% acetonitrile in water, plus 0.1% TFA) to afford the title compound (27 mg, 0.065 mmol, 7.68% yield); white powder. ¹H-NMR (400 MHz, CHLOROFORM-d) δ ppm 15.46 (br. s., 1 H), 9.95 (t, J=5.05 Hz, 1 H), 7.36 (d, J=8.34 Hz, 2 H), 7.06 (d, J=8.08 Hz, 2 H), 5.29 (br. s., 2 H), 4.24 (d, J=5.31 Hz, 2 H), 2.66 (d, J=6.57 Hz, 2 H), 1.30 (s, 9 H), 1.09-1.21 (m, 2 H), 0.89 (t, J=6.82 Hz, 1 H), 0.55-0.63 (m, 2 H). LCMS (ES⁺) m/z 414 (MH⁺).

### EXAMPLE 54

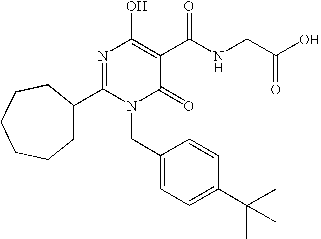

### N-[(2-Cycloheptyl-1-{[4-(1,1-dimethylethyl)phenyl]methyl}-4-hydroxy-6-oxo-1,6-dihydro-5-pyrimidinyl)carbonyl]glycine

54a) 2-Cycloheptyl-3-{[4-(1,1-dimethylethyl)phenyl]methyl}-6-hydroxy-4(3H)-pyrimidinone. Dimethylaluminium chloride (1.212 ml, 1.212 mmol) was added to a solution of 2-cycloheptylnitrile (0.170 ml, 1.322 mmol) and 4-t-butylbenzylamine (0.194 ml, 1.102 mmol) in toluene (1.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (4.0 ml). Diethylmalonate (0.668 ml, 4.41 mmol) and sodium methoxyde (1.008 ml, 4.41 mmol) were added and the mixture was stirred at reflux for 20 h. After cooling, the mixture was poured into water. The pH was adjusted to ca. 3 by the addition of 1 N HCl. The resulting precipitate was collected, washed with water and dried to afford 2-cycloheptyl-3-{[4-(1,1-dimethylethyl)phenyl]methyl}-6-hydroxy-4(3H)-pyrimidinone (305 mg, 0.860 mmol, 78.1% yield; 90% pure by LC/MS). This product was used in the next step as is. LCMS (ES⁺) m/z 355 (MH⁺).

54b) N-[(2-Cycloheptyl-1-{[4-(1,1-dimethylethyl)phenyl]methyl}-4-hydroxy-6-oxo-1,6-dihydro-5-pyrimidinyl)carbonyl]glycine. A solution of 2-cycloheptyl-3-{[4-(1,1-dimethylethyl)phenyl]methyl}-6-hydroxy-4(3H)-pyrimidinone (300 mg, 0.85 mmol), Hunig's base (0.296 mL, 1.70 mmol) and ethyl isocyanatoacetate (0.191 mL, 1.70 mmol) in dichloromethane (DCM) (3.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (4.5 mL) and 1 M NaOH (4.5 mL, 4.5 mmol) and stirred at rt for 3 h. It was then poured into water and acidified by the addition of 6 N HCl. The precipitate was collected, washed with water and dried to give 225 mg of crude material. Purification by RP-HPLC (15 to 100% acetonitrile in water, plus 0.1% TFA) afforded the title compound (42 mg, 0.092 mmol, 10.85% yield); yellow powder. ¹H-NMR (400 MHz, DMSO-d₆) δ ppm 15.80 (br. s., 1 H), 9.83 (t, J=5.68 Hz, 1 H), 7.38 (d, J=8.34 Hz, 2 H), 7.12 (d, J=8.34 Hz, 2 H), 5.32 (br. s., 2 H), 4.07 (d, J=5.81 Hz, 2 H), 2.88-3.02 (m, 1 H), 1.54-1.71 (m, 6 H), 1.38-1.54 (m, 4 H), 1.25 (s, 9 H), 1.18-1.33 (m, 2 H). LCMS (ES⁺) m/z 456 (MH⁺).

### EXAMPLE 55

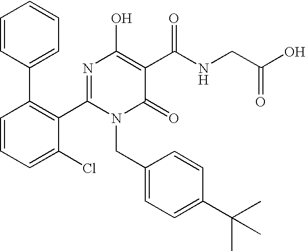

### N-[(2-(3-Chloro-2-biphenylyl)-1-{[4-(1,1-dimethylethyl)phenyl]methyl}-4-hydroxy-6-oxo-1,6-dihydro-5-pyrimidinyl)carbonyl]glycine

55a) 3-Chloro-2-biphenylamine. A mixture of 2-chloro-6-iodoaniline (0.507g, 2.00 mmol), phenylboronic acid (0.244 g, 2.00 mmol), tetrakis(triphenylphosphine)palladium (0) (0.115 g, 0.100 mmol), 2 M aqueous sodium carbonate (2 mL, 4 mmol) and dioxane (4 mL) was stirred in a microwave reactor at 160° C. for 0.5 h under argon, then cooled and poured into 0.1 M aqueous sodium hydroxide (40 mL). The mixture was extracted with ether and the extracts washed with water, brine, dried (Na₂SO₄) and evaporated under reduced pressure. The residue was chromatographed (silica gel, 1-10% ethyl acetate/hexane) to give the title compound (0.335 g, 82%) as a white, waxy solid. 1H NMR (400 MHz, CHLOROFORM-d) δ ppm 2.94 (br. s., 2 H) 6.78 (t, J=7.83 Hz, 1 H) 7.05 (dd, J=7.45, 1.39 Hz, 1 H) 7.29 (dd, J=8.08, 1.52 Hz, 1 H) 7.36-7.43 (m, 1 H) 7.43-7.52 (m, 4 H).

55b) 3-Chloro-2-biphenylcarbonitrile. tert-Butyl nitrite (0.582 mL, 4.89 mmol) was injected over 5 min into a stirred solution of 3-chloro-2-biphenylamine (0.333 g, 1.63 mmol) and copper (I) cyanide (0.191 g, 2.13 mmol) in dimethylsulfoxide (8 mL) at 50° C. under argon. The mixture was stirred for 1.5 h at 50° C., then cooled, poured into 1 M aqueous hydrochloric acid (80 mL) and extracted with ethyl acetate. The extracts were washed with water, brine, then dried (MgSO₄). The solvent was evaporated under reduced pressure and the residue chromatographed (silica gel, 2-30% ethyl acetate/hexane) to give the title compound (0.174 g, 50%) as a pale orange solid. 1H NMR (400 MHz, DMSO-d₆) δ ppm 7.50-7.64 (m, 6 H) 7.76-7.84 (m, 2 H).

51c) 2-(3-Chloro-2-biphenylyl)-3-{[4-(1,1-dimethylethyl)phenyl]methyl}-6-hydroxy-4(3H)-pyrimidinone. A 1 M solution of dimethylaluminium chloride in hexane (0.369 mL, 0.369 mmol) was added to a stirred mixture of 4-tert-butylbenzylamine (0.055 g, 0.336 mmol), 3-chloro-2-biphenylcarbonitrile (0.086 g, 0.403 mmol) and toluene (1 mL) at room temperature. The mixture was stirred in a microwave reactor at 150° C. for 0.5 h, then cooled and the solvent removed under reduced pressure. Diethyl malonate (0.215 g, 1.34 mmol), 2-methoxyethanol (3 mL) and 4.37 M sodium methoxide in methanol (0.308 mL, 1.34 mmol) were added and the mixture refluxed under nitrogen for 20 h. After cooling, the mixture was poured into water (50 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was chromatographed (silica gel, 1-9% methanol/dichloromethane) to give the title compound (0.049 g, 33%) as a gum. 1H NMR (400 MHz, DMSO-d₆) δ ppm 1.22 (s, 9 H) 4.35 (d, J=15.16 Hz, 1 H) 4.70 (d, J=15.16 Hz, 1 H) 5.44 (s, 1 H) 6.57-6.63 (m, 2 H) 7.10-7.21 (m, 4 H) 7.27-7.38 (m, 3 H) 7.47 (dd, J=7.58, 1.01 Hz, 1 H) 7.55 (dd, J=8.08, 1.01 Hz, 1 H) 7.66 (t, J=7.96 Hz, 1 H) 11.77 (br. s., 1 H).

51d) N-[(2-(3-Chloro-2-biphenylyl)-1-{[4-(1,1-dimethylethyl)phenyl]methyl}-4-hydroxy-6-oxo-1,6-dihydro-5-pyrimidinyl)carbonyl]glycine. A mixture of 2-(3-chloro-2-biphenylyl)-3-{[4-(1,1-dimethylethyl)phenyl]methyl}-6-hydroxy-4(3H)-pyrimidinone (0.048 g, 0.108 mmol), ethyl 2-isocyanatoacetate (0.036 mL, 0.324 mmol), N,N-diisopropylethylamine (0.056 mL, 0.324 mmol) and dichloromethane (0.5 mL) was stirred in a microwave reactor at 130° C. for 1 h, then cooled. 1 M aqueous hydrochloric acid (2 mL) was added and the mixture extracted with dichloromethane. The extracts were dried (MgSO₄), evaporated under reduced pressure to give the intermediate ester. 1 M aqueous sodium hydroxide (1.00 mL, 1.00 mmol) was added dropwise to a stirred solution of the intermediate ester in ethanol (5 mL) and the mixture stirred for 18 h at room temperature, then diluted with water (50 mL) and washed with ether. 6 M aqueous hydrochloric acid was added to adjust the pH to 1 and the mixture extracted with ethyl acetate. The extracts were washed with water, brine, dried (MgSO₄) and evaporated under reduced pressure. The residue was purified by HPLC (ODS, 20-100% acetonitrile/water+0.1% trifluoroacetic acid) to give the title compound (0.028 g, 47%) as a foam. 1H NMR (400 MHz, DMSO-d₆) δ ppm 1.22 (s, 9 H) 4.07 (d, J=5.56 Hz, 2 H) 4.53 (d, J=15.16 Hz, 1 H) 4.74 (d, J=15.41 Hz, 1 H) 6.62-6.69 (m, 2 H) 7.14-7.21 (m, 4 H) 7.29-7.39 (m, 3 H) 7.51 (dd, J=7.58, 1.01 Hz, 1 H) 7.62 (dd, J=8.08, 1.01 Hz, 1 H) 7.71 (t, J=7.96 Hz, 1 H) 9.71 (t, J=5.68 Hz, 1 H) 12.94 (br. s., 1 H) 16.22 (s, 1 H).

### EXAMPLE 56

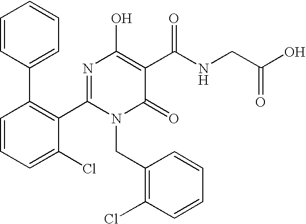

### N-({2-(3-Chloro-2-biphenylyl)-1-[(2-chlorophenyl)methyl]-4-hydroxy-6-oxo-1,6-dihydro-5-pyrimidinyl}carbonyl)glycine

56a) 2-(3-Chloro-2-biphenylyl)-3-[(2-chlorophenyl)methyl]-6-hydroxy-4(3H)-pyrimidinone. 1 M solution of dimethylaluminium chloride in hexane (0.369 mL, 0.369 mmol) was added to a stirred mixture of 2-chlorobenzylamine (0.048 g, 0.336 mmol), 3-chloro-2-biphenylcarbonitrile (example 43(b), 0.086 g, 0.403 mmol) and toluene (1 mL) at room temperature. The mixture was stirred in a microwave reactor at 150° C. for 0.5 h, then cooled and the solvent removed under reduced pressure. Diethyl malonate (0.215 g, 1.34 mmol), 2-methoxyethanol (3 mL) and 4.37 M sodium methoxide in methanol (0.308 mL, 1.34 mmol) were added and the mixture refluxed under nitrogen for 20 h. After cooling, the mixture was poured into water (50 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was chromatographed (silica gel, 1-9% methanol/dichloromethane) to give the title compound (0.062 g, 44%) as a gum. 1H NMR (400 MHz, DMSO-d₆) δ ppm 4.45 (d, J=16.17 Hz, 1 H) 4.86 (d, J=15.92 Hz, 1 H) 5.52 (s, 1 H) 6.66 (dd, J=7.83, 1.01 Hz, 1 H) 7.09 (td, J=7.52, 1.39 Hz, 1 H) 7.21 (td, J=7.71, 1.52 Hz, 1 H) 7.24-7.31 (m, 3 H) 7.35-7.42 (m, 3 H) 7.48 (dd, J=7.71, 1.14 Hz, 1 H) 7.51 (dd, J=8.08, 1.01 Hz, 1 H) 7.62 (t, J=7.96 Hz, 1 H) 11.98 (s, 1 H).

56b) N-({2-(3-Chloro-2-biphenylyl)-1-[(2-chlorophenyl)methyl]-4-hydroxy-6-oxo-1,6-dihydro-5-pyrimidinyl}carbonyl)glycine. A mixture of 2-(3-chloro-2-biphenylyl)-3-[(2-chlorophenyl)methyl]-6-hydroxy-4(3H)-pyrimidinone (0.061 g, 0.144 mmol), ethyl 2-isocyanatoacetate (0.049 mL, 0.432 mmol), N,N-diisopropylethylamine (0.075 mL, 0.432 mmol) and dichloromethane (0.5 mL) was stirred in a microwave reactor at 130° C. for 1 h, then cooled. 1 M aqueous hydrochloric acid (2 mL) was added and the mixture extracted with dichloromethane. The extracts were dried (MgSO₄), evaporated under reduced pressure to give the intermediate ester. 1 M aqueous sodium hydroxide (1.00 mL, 1.00 mmol) was added dropwise to a stirred solution of the intermediate ester in ethanol (5 mL) and the mixture stirred for 18 h at room temperature, then diluted with water (50 mL) and washed with ether. 6 M aqueous hydrochloric acid was added to adjust the pH to 1 and the mixture extracted with ethyl acetate. The extracts were washed with water, brine, dried (MgSO₄) and evaporated under reduced pressure. The residue was purified by HPLC (ODS, 20-100% acetonitrile/water+0.1% trifluoroacetic acid) to give the title compound (0.026 g, 34%) as a foam. 1H NMR (400 MHz, DMSO-d₆) δ ppm 4.08 (d, J=5.56 Hz, 2 H) 4.57 (d, J=16.17 Hz, 1 H) 4.95 (d, J=16.17 Hz, 1 H) 6.67 (d, J=7.58 Hz, 1 H) 7.11 (td, J=7.58, 1.01 Hz, 1 H) 7.19-7.30 (m, 3 H) 7.32 (dd, J=8.08, 1.01 Hz, 1 H) 7.37-7.44 (m, 3 H) 7.52 (d, J=7.58 Hz, 1 H) 7.56 (dd, J=8.08, 0.76 Hz, 1 H) 7.67 (t, J=7.96 Hz, 1 H) 9.67 (t, J=5.43 Hz, 1 H) 12.96 (br. s., 1 H) 16.37 (s, 1 H).

### EXAMPLE 57

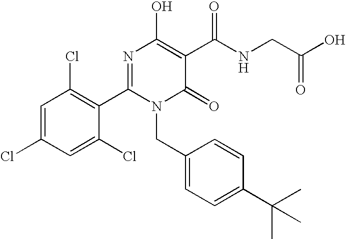

### N-{[1-{[4-(1,1-Dimethylethyl)phenyl]methyl}-4-hydroxy-6-oxo-2-(2,4,6-trichlorophenyl)-1,6-dihydro-5-pyrimidinyl]carbonyl}glycine

57a) 3-{[4-(1,1-Dimethylethyl)phenyl]methyl}-6-hydroxy-2-(2,4,6-trichlorophenyl)-4(3H)-pyrimidinone. A 1 M solution of dimethylaluminium chloride in hexane (2.75 mL, 2.75 mmol) was added to a stirred mixture of 4-tert-butylbenzylamine (0.408 g, 2.50 mmol), 2,4,6-trichlorobenzonitrile (0.619 g, 3.00 mmol) and toluene (3 mL) at room temperature. After 2 min at room temperature, the mixture was stirred in a microwave reactor at 150° C. for 0.5 h, then cooled and the solvent removed under reduced pressure. Diethyl malonate (1.60 g, 10.0 mmol), 2-methoxyethanol (15 mL) and 4.37 M sodium methoxide in methanol (2.30 mL, 10.0 mmol) were added and the mixture refluxed under nitrogen for 8 h. After cooling, the mixture was poured into water (150 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.551 g, 50%) as a solid. 1H NMR (400 MHz, DMSO-d₆) δ ppm 1.23 (s, 9 H) 4.88 (s, 2 H) 5.59 (s, 1 H) 6.79-6.85 (m, 2 H) 7.18-7.25 (m, 2 H) 7.84 (s, 2 H) 11.94 (br. s., 1 H).

57b) N-{[1-{[4-(1,1-Dimethylethylphenyl]methyl}-4-hydroxy-6-oxo-2-(2,4,6-trichlorophenyl)-1,6-dihydro-5-pyrimidinyl]carbonyl}glycine disodium salt. A mixture of 3-{[4-(1,1-dimethylethyl)phenyl]methyl}-6-hydroxy-2-(2,4,6-trichlorophenyl)-4(3H)-pyrimidinone (0.548 g, 1.25 mmol), ethyl 2-isocyanatoacetate (0.281 mL, 2.50 mmol), N,N-diisopropylethylamine (0.435 mL, 2.50 mmol) and dichloromethane (4 mL) was stirred in a microwave reactor at 130° C. for 1 h, then cooled. Trifluoroacetic acid (0.2 mL, 2.6 mmol) was added and the mixture chromatographed (silica gel, 1-5% methanol/dichloromethane) to give the intermediate ester. 1 M aqueous sodium hydroxide (6.00 mL, 6.00 mmol) was added dropwise to a stirred solution of the intermediate ester in ethanol (24 mL) and the mixture stirred for 2 h at room temperature. The precipitate was filtered, washed with 10% aqueous ethanol, then ethanol and dried to give the title compound (0.300 g, 41%) as a white solid. 1H NMR (400 MHz, DMSO-d₆) δ ppm 1.23 (s, 9 H) 3.48 (d, J=4.04 Hz, 2 H) 4.74 (s, 2 H) 6.81-6.87 (m, 2 H) 7.15-7.21 (m, 2 H) 7.72 (s, 2 H) 10.09 (t, J=4.04 Hz, 1 H).

### EXAMPLE 58

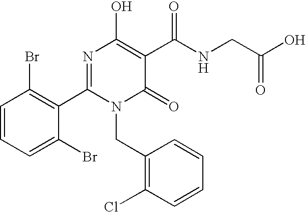

### N-{[1-[(2-Chlorophenyl)methyl]-2-(2,6-dibromophenyl)-4-hydroxy-6-oxo-1,6-dihydro-5-pyrimidinyl]carbonyl}glycine

58a) 3-[(2-Chlorophenyl)methyl]-2-(2,6-dibromophenyl)-6-hydroxy-4(3H)-pyrimidinone. A 1 M solution of dimethylaluminium chloride in hexane (1.10 mL, 1.10 mmol) was added to a stirred mixture of 2-chlorobenzylamine (0.142 g, 1.00 mmol), 2,6-dibromobenzonitrile (example 32(a), 0.313 g, 1.20 mmol) and toluene (1 mL) at room temperature. The mixture was stirred in a microwave reactor at 150° C. for 0.5 h, then cooled and the solvent removed under reduced pressure. Diethyl malonate (0.533 g, 3.33 mmol), 2-methoxyethanol (4 mL) and 4.37 M sodium methoxide in methanol (0.767 mL, 3.33 mmol) were added and the mixture refluxed under nitrogen for 5 h. Periodically, the reaction was flushed with argon to remove more volatile solvent. After cooling, the mixture was poured into water (75 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.261 g, 55%) as a tan solid. 1H NMR (400 MHz, DMSO-d₆) 6 ppm 5.07 (s, 2 H) 5.65 (s, 1 H) 7.11-7.18 (m, 1 H) 7.23-7.33 (m, 3 H) 7.39 (t, J=8.21 Hz, 1 H) 7.75 (d, J=8.08 Hz, 2 H) 12.04 (br. s., 1 H).

58b) N-{[1-[(2-Chlorophenyl)methyl]-2-(2,6-dibromophenyl)-4-hydroxy-6-oxo-1,6-dihydro-5-pyrimidinyl]carbonyl}glycine. A mixture of 3-[(2-chlorophenyl)methyl]-2-(2,6-dibromophenyl)-6-hydroxy-4(3H)-pyrimidinone (0.258 g, 0.547 mmol), ethyl 2-isocyanatoacetate (0.123 mL, 1.09 mmol), N,N-diisopropylethylamine (0.191 mL, 1.09 mmol) and dichloromethane (2 mL) was stirred in a microwave reactor at 130° C. for 1 h, then cooled. Trifluoroacetic acid (0.2 mL, 2.6 mmol) was added and the mixture chromatographed (silica gel, 1-5% methanol dichloromethane) to give the intermediate ester. 1 M aqueous sodium hydroxide (2.00 mL, 2.00 mmol) was added dropwise to a stirred solution of the intermediate ester in ethanol (10 mL) and the mixture stirred for 20 h. 6 M aqueous hydrochloric acid was added to adjust the pH to 1 and the mixture diluted with water (50 mL) and stirred 0.5 h. The precipitate was filtered, washed with water and dried to give the title compound (0.139 g, 44%) as a cream powder. 1H NMR (400 MHz, DMSO-d₆) δ ppm 4.14 (d, J=5.81 Hz, 2 H) 5.19 (s, 2 H) 7.13-7.19 (m, 1 H) 7.24-7.37 (m, 3) 7.44 (t, J−8.08 Hz, 1 H) 7.79 (d, J=8.08 Hz, 2 H) 9.86 (t, J=5.68 Hz, 1 H) 13.00 (br. s., 1 H).

### EXAMPLE 59

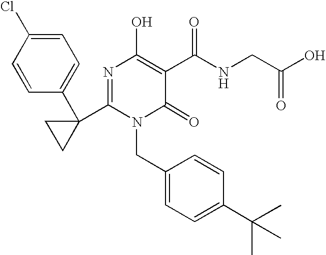

### N-[(2-{[1-(4-Chlorophenyl)cyclopropyl]-1-{[4-(1,1-dimethylethyl)phenyl]methyl}-4-hydroxy-6-oxo-1,6-dihydro-5-pyrimidinyl)carbonyl]glycine

59a) 2-[1-(4-Chlorophenyl)cyclopropyl]-3-{[4-(1,1-dimethylethyl)phenyl]methyl}-6-hydroxy-4(3H)-pyrimidinone. Dimethylaluminium chloride (1.212 ml, 1.212 mmol) was added to a solution of 1-(4-chlorophenyl)cyclopropanecarbonitrile (234 mg, 1.322 mmol) and 4-t-butylbenzylamine (0.194 ml, 1.102 mmol) in toluene (1.5 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (4.0 ml). Diethylmalonate (0.668 ml, 4.41 mmol) and sodium methoxyde (1.008 ml, 4.41 mmol) were added and the mixture was stirred at reflux for 20 h. After cooling, the mixture was poured into water. The pH was adjusted to ca. 5 by the addition of 1 N HCl. The resulting precipitate was collected, washed with water, dried and purified on silica gel (0-9% MeOH in chloroform) to afford 2-[1-(4-chlorophenyl)cyclopropyl]-3-{[4-(1,1-dimethylethyl)phenyl]methyl}-6-hydroxy-4(3H)-pyrimidinone (387 mg, 0.946 mmol, 84.5% yield). ¹H-NMR (400 MHz, CHLOROFORM-d) d ppm 7.13-7.23 (m, 4 H), 6.97 (d, J=8.59 Hz, 2 H), 6.79 (d, J=8.34 Hz, 2 H), 5.79 (s, 1 H), 5.25 (br. s., 2 H), 1.38-1.44 (m, 2 H), 1.29-1.34 (m, 2 H), 1.27 (s, 9 H). LCMS (ES⁺) m/z 409 (MH⁻).

59b) N-[(2-[1-(4-Chlorophenyl)cyclopropyl]-1-{[4-(1,1-dimethylethyl)phenyl]methyl}-4-hydroxy-6-oxo-1,6-dihydro-5-pyrimidinyl)carbonyl]glycine. A solution of 2-[1-(4-chlorophenyl)cyclopropyl]-3-{[4-(1,1-dimethylethyl)phenyl]methyl}-6-hydroxy-4(3H)-pyrimidinone (215 mg, 0.526 mmol), Hunig's base (0.183 mL, 1.05 mmol) and ethyl isocyanatoacetate (0.118 mL, 1.05 mmol) in Dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (4.5 mL) and 1 M NaOH (4.5 mL, 4.5 mmol) and stirred at rt for 4.5 h. It was then puored into water and acidified by the addition of 6 N HCl. The precipitate was collected, washed with water and dried to give 175 mg of crude material. Purification by RP-HPLC (20 to 95% Acetonitrile in water, plus 0.1% TFA) afforded the title compound (115 mg, 0.225 mmol, 42.87% yield); white powder. ¹H-NMR (400 MHz, DMSO-d₆) d ppm 15.94 (br. s., 1 H), 9.76 (t, J=5.56 Hz, 1 H), 7.07-7.18 (m, 6 H), 6.70 (d, J=8.34 Hz, 2 H), 5.18 (s, 2 H), 4.05 (d, J=5.56 Hz, 2 H), 1.54-1.65 (m, 2 H), 1.35-1.45 (m, 2 H), 1.22 (s, 9 H). LCMS (ES⁻) m/z 510 (MH⁺).

### EXAMPLE 60

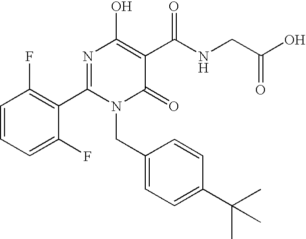

### N-[(2-(2,6-Difluorophenyl)-1-{[4-(1,1-dimethylethyl)phenyl]methyl}-4-hydroxy-6-oxo-1,6-dihydro-5-pyrimidinyl)carbonyl]glycine

60a) 2-(2,6-Difluorophenyl)-3-{[4-(1,1-dimethylethylphenyl]methyl}-6-hydroxy-4(3H)-pyrimidinone. A 1 M solution of dimethylaluminium chloride in hexane (1.10 mL, 1.10 mmol) was added to a stirred mixture of 4-tert-butylbenzylamine (0.163 g, 1.00 mmol), 2,6-difluorobenzonitrile (0.167 g, 1.20 mmol) and toluene (1 mL) at room temperature. The mixture was stirred in a microwave reactor at 150° C. for 0.5 h, then cooled and the solvent removed under reduced pressure. Diethyl malonate (0.533 g, 3.33 mmol), 2-methoxyethanol (4 mL) and 4.37 M sodium methoxide in methanol (0.767 mL, 3.33 mmol) were added and the mixture refluxed under nitrogen for 6 h. After cooling, the mixture was poured into water (50 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.156 g, 42%) as a cream solid. 1H NMR (400 MHz, DMSO-d₆) δ ppm 1.22 (s, 9 H) 4.96 (s, 2 H) 5.58 (s, 1 H) 6.72-6.79 (m, 2 H) 7.18-7.28 (m, 4 H) 7.59-7.72 (m, 1 H) 11.92 (br. s., 1 H).

60b) N-[(2-(2,6-Difluorophenyl)-1-{[4-(1,1-dimethylethyl)phenyl]methyl}-4-hydroxy-6-oxo-1,6-dihydro-5-pyrimidinyl)carbonyl]glycine. A mixture of 2-(2,6-difluorophenyl)-3-{[4-(1,1-dimethylethyl)phenyl]methyl}-6-hydroxy-4(3H)-pyrimidinone (0.153 g, 0.413 mmol), ethyl 2-isocyanatoacetate (0.093 mL, 0.826 mmol), N,N-diisopropylethylamine (0.144 mL, 0.826 mmol) and dichloromethane (1 mL) was stirred in a microwave reactor at 130° C. for 1 h, then cooled. Trifluoroacetic acid (0.2 mL, 2.6 mmol) was added and the mixture chromatographed (silica gel, 1-5% methanol/dichloromethane) to give the intermediate ester. 1 M aqueous sodium hydroxide (3.00 mL, 3.00 mmol) was added dropwise to a stirred solution of the intermediate ester in ethanol (6 mL) and the mixture stirred for 1 h, then diluted with water (40 mL) and acidified with 6 M aqueous hydrochloric acid (1 mL). After stirring 0.5 h, the precipitate was filtered, washed with water and dried to give the title compound (0.085 g, 44%) as a white solid. 1H NMR (400 MHz, DMSO-d₆) δ ppm 1.22 (s, 9 H) 4.12 (d, J=5.81 Hz, 2 H) 5.07 (s, 2 H) 6.84 (d, J=8.34 Hz, 2 H) 7.22-7.34 (m, 4 H) 7.64-7.77 (m, 1 H) 9.83 (t, J=5.43 Hz, 1 H) 12.99 (br. s., 1 H).

### EXAMPLE 61

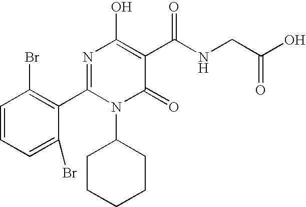

### N-{[1-Cyclohexyl-2-(2,6-dibromophenyl)-4-hydroxy-6-oxo-1,6-dihydro-5-pyrimidinyl]carbonyl}glycine

61a) 3-Cyclohexyl-2-(2,6-dibromophenyl)-6-hydroxy-4(3H)-pyrimidinone. A 1 M solution of dimethylaluminium chloride in hexane (1.10 mL, 1.10 mmol) was added to a stirred mixture of cyclohexylamine (0.099 g, 1.00 mmol), 2,6-dibromobenzonitrile (example 32(a), 0.313 g, 1.20 mmol) and toluene (1 mL) at room temperature. The mixture was stirred in a microwave reactor at 150° C. for 0.5 h, then cooled and the solvent removed under reduced pressure. Diethyl malonate (0.533 g, 3.33 mmol), 2-methoxyethanol (4 mL) and 4.37 M sodium methoxide in methanol (0.767 mL, 3.33 mmol) were added and the mixture refluxed under nitrogen for 18 h. After cooling, the mixture was poured into water (75 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was triturated with ether and the solid collected, washed with ether and dried to give the title compound (0.185 g, 43%) as a cream solid. 1H NMR (400 MHz, DMSO-d₆) δ ppm 0.79-0.95 (m, 2 H) 0.98-1.13 (m, 1 H) 1.43-1.55 (m, 1 H) 1.69-1.76 (m, 2 H) 1.79-1.89 (m, 2 H) 2.51-2.61 (m, 2 H) 3.32-3.40 (m, 1 H) 5.41 (s, 1 H) 7.44 (t, J=8.08 Hz, 1 H) 7.87 (d, J=8.08 Hz, 2 H) 11.68 (br. s., 1 H).

61b) N-{[1-Cyclohexyl-2-(2,6-dibromophenyl)-4-hydroxy-6-oxo-1,6-dihydro-5-pyrimidinyl]carbonyl}glycine. A mixture of 3-cyclohexyl-2-(2,6-dibromophenyl)-6-hydroxy-4(3H)-pyrimidinone (0.183 g, 0.427 mmol), ethyl 2-isocyanatoacetate (0.096 mL, 0.855 mmol), N,N-diisopropylethylamine (0.149 mL, 0.855 mmol) and dichloromethane (1 mL) was stirred in a microwave reactor at 130° C. for 1 h, then cooled. Trifluoroacetic acid (0.1 mL, 1.3 mmol) was added and the mixture chromatographed (silica gel, 1-5% methanol/dichloromethane) to give the intermediate ester. 1 M aqueous sodium hydroxide (3.00 mL, 3.00 mmol) was added dropwise to a stirred solution of the intermediate ester in ethanol (6 mL) and the mixture stirred for 2 h, then diluted with water (60 mL), acidified to pH 1 with 6 M aqueous hydrochloric acid and stirred 0.25 h. The precipitate was filtered, washed with water and dried to give the title compound (0.116 g, 51%) as a white solid. 1H NMR (400 MHz, DMSO-d₆) δ ppm 0.86-0.99 (m, 2 H) 1.02-1.12 (m, 1 H) 1.48-1.60 (m, 1 H) 1.75 (m, 2 H) 1.87-2.00 (m, 2 H) 2.48-2.60 (m, 1 H) 4.11 (d, J=5.81 Hz, 2 H) 7.50 (t, J=8.08 Hz, 1 H) 7.92 (d, J=8.08 Hz, 2 H) 9.89 (t, J=5.43 Hz, 1 H) 12.97 (br. s., 1 H) 16.28 (s, 1 H).

### EXAMPLE 62

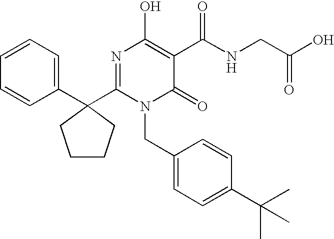

### N-{[1-{[4-(1,1-Dimethylethyl)phenyl]methyl}-4-hydroxy-6-oxo-2-(1-phenylcyclopentyl)-1,6-dihydro-5-pyrimidinyl]carbonyl}glycine

Dimethylaluminium chloride (1.212 ml, 1.212 mmol) was added to a solution of 1-phenylcyclopentanecarbonitrile (226 mg, 1.322 mmol) and 4-t-butylbenzylamine (0.194 ml, 1.102 mmol) in toluene (1.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (4.0 ml). Diethylmalonate (0.668 ml, 4.41 mmol) and sodium methoxyde (1.008 ml, 4.41 mmol) were added and the mixture was stirred at reflux for 20 h. After cooling, the mixture was poured into water. The pH was adjusted to ca. 6 by the addition of 6 N HCl and the mixture was extracted with EtOAc (2×). The organic extracts were washed with brine, dried over Na₂SO₄ and evaporated. The residue was purified on silica gel (0-9% MeOH in chloroform) to afford 3-{[4-(1,1-dimethylethyl)phenyl]methyl}-6-hydroxy-2-(1-phenylcyclopentyl)-4(3H)-pyrimidinone (315 mg, red oil; mixture of desired product and other impurities).

A solution of the above product (300 mg, 0.74 mmol), Hunig's base (0.260 mL, 1.49 mmol) and ethyl isocyanatoacetate (0.167 mL, 1.49 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (5 mL) and 1 M NaOH (5 mL, 5 mmol) and stirred at rt for 5 h. The ethanol was evaporated under reduced pressure, the pH was adjusted to ˜5 with 1 N HCl and the mixture was extracted with EtOAc (2×). The organic extracts were washed with brine, dried over Na₂SO₄ and evaporated. The residue was purified by RP-HPLC (15 to 95% acetonitrile in water, plus 0.1% TFA) afforded the title compound (70 mg, 0.139 mmol, 12.61% yield) contaminated by traces of methyl N-{[1-{[4-(1,1-dimethylethyl)phenyl]methyl}-4-hydroxy-6-oxo-2-(1-phenylcyclopentyl)-1,6-dihydro-5-pyrimidinyl]carbonyl}glycinate, derived by azeotroping the aqueos fractions in the presence of methanol. Thirty-eight mg of this mixture was dissolved in EtOH (4 mL) and 1 M NaOH (4 mL, 4 mmol) and stirred at rt for 2.5 h. After diluting with water, the pH was adjusted to 4 by the addition of 1N HCl. The precipitate was collected, washed with water and dried to afford N-{[1-{[4-(1,1-dimethylethyl)phenyl]methyl}-4-hydroxy-6-oxo-2-(1-phenylcyclopentyl)-1,6-dihydro-5-pyrimidinyl]carbonyl}glycine (30 mg; white powder. ¹H-NMR (400 MHz, CHLOROFORM-d) d ppm 15.31 (s, 1 H), 9.87 (t,J=5.68 Hz, 1 H), 7.28-7.33 (m, 2 H), 7.19-7.26 (m, 3 H), 7.12-7.17 (m, 2 H), 6.70 (d, J=8.34 Hz, 2 H), 4.86 (s, 2 H), 4.19 (d, J=5.56 Hz, 2 H), 2.37-2.57 (m, 2 H), 2.05-2.18 (m, 2 H), 1.61-1.84 (m, 4 H), 1.27 (s, 9 H). LCMS (ES⁻) m/z 504 (MH⁺).

### EXAMPLE 63

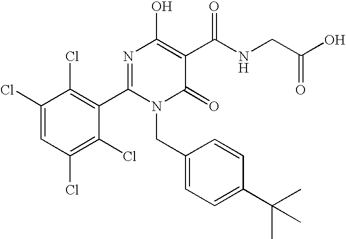

### N-{[1-{[4-(1,1-Dimethylethyl)phenyl]methyl}-4-hydroxy-6-oxo-2-(2,3,5,6-tetrachlorophenyl)-1,6-dihydro-5-pyrimidinyl]carbonyl}glycine

63a) 2,3,5,6-Tetrachlorobenzonitrile. tert-Butyl nitrite (1.58 mL, 13.3 mmol) was injected dropwise into a stirred solution of 2,3,5,6-tetrachloroaniline (1.02 g, 4.42 mmol) and copper (I) cyanide (0.514 g, 5.74 mmol) in dimethylsulfoxide (20 mL) at 50° C. under argon. The mixture was stirred for 2 h at 50° C., then cooled, poured into 0.1 M aqueous hydrochloric acid (100 mL). The solid was filtered, washed with water, then dissolved in ethyl acetate. The solution was filtered, dried (MgSO₄) and evaporated under reduced pressure. The residue was chromatographed (silica gel, 5-40% ethyl acetate/hexane) to give the title compound (0.672 g, 63%) as a cream solid. 1H NMR (400 MHz, CHLOROFORM-D) δ ppm 7.59 (s, 1 H).

63b) 3-{[4-(1,1-Dimethylethyl)phenyl]methyl}-6-hydroxy-2-(2,3,5,6-tetrachlorophenyl)-4(3H)-pyrimidinone. A 1 M solution of dimethylaluminium chloride in hexane (1.10 mL, 1.10 mmol) was added to a stirred mixture of 4-tert-butylbenzylamine (0.163 g, 1.00 mmol), 2,3,5,6-tetrachlorobenzonitrile (0.288 g, 1.20 mmol) and toluene (1 mL) at room temperature. The mixture was stirred in a microwave reactor at 150° C. for 0.5 h, then cooled and the solvent removed under reduced pressure. Diethyl malonate (0.533 g, 3.33 mmol), 2-methoxyethanol (4 mL) and 4.37 M sodium methoxide in methanol (0.767 mL, 3.33 mmol) were added and the mixture refluxed under nitrogen for 4 h. Periodically, the reaction was flushed with argon to remove more volatile solvent. After cooling, the mixture was poured into water (50 mL), washed with ether, acidified to pH 1 with 6 M aqueous hydrochloric acid, and extracted with ethyl acetate. The extracts were washed with water and brine, dried (MgSO₄) and evaporated under reduced pressure. The residue was chromatographed (silica gel, 1-9% methanol/dichloromethane) to give the title compound (0.220 g, 47%) as a gum. 1H NMR (400 MHz, DMSO-d₆) δ ppm 1.24 (m, 9 H) 4.90 (s, 2 H) 5.63 (s, 1 H) 6.71-6.78 (m, 2 H) 7.16-7.21 (m, 2H) 8.35 (s, 1 H) 11.98 (s, 1 H).

63c) N-{[1-{[4-(1,1-Dimethylethyl)phenyl[methyl}-4-hydroxy-6-oxo-2-(2,3,5,6-tetrachlorophenyl)-1,6-dihydro-5-pyrimidinyl]carbonyl}glycine. A mixture of 3-{[4-(1,1-dimethylethyl)phenyl]methyl}-6-hydroxy-2-(2,3,5,6-tetrachlorophenyl)-4(3H)-pyrimidinone (0.210 g, 0.445 mmol), ethyl 2-isocyanatoacetate (0.100 mL, 0.889 mmol), N,N-diisopropylethylamine (0.155 mL, 0.889 mmol) and dichloromethane (1 mL) was stirred in a microwave reactor at 140° C. for 1 h, then cooled. Trifluoroacetic acid (0.2 mL, 2.6 mmol) was added and the mixture chromatographed (silica gel, 1-5% methanol/dichloromethane) to give the intermediate ester. 1 M aqueous sodium hydroxide (2.00 mL, 2.00 mmol) was added dropwise to a stirred solution of the intermediate ester in ethanol (8 mL) and the mixture stirred for 18 h, then diluted with water (70 mL), acidified with 6 M aqueous hydrochloric acid (1 mL) and stirred 0.25 h. The precipitate was filtered, washed with water and dried to give the title compound (0.085 g, 33%) as a white powder. 1H NMR (400 MHz, DMSO-d₆) δ ppm 1.24 (s, 9 H) 4.15 (d, J=5.81 Hz, 2 H) 5.02 (s, 2 H) 6.84 (d, J=8.34 Hz, 2 H) 7.23 (d, J=8.34 Hz, 2 H) 8.38 (s, 1 H) 9.88 (t, J=5.68 Hz, 1 H) 13.00 (br. s., 1 H).

### EXAMPLE 64

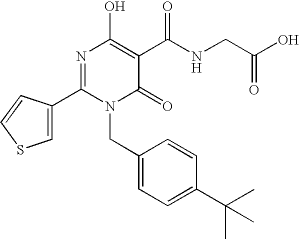

### N-{[1-{[4-(1,1-Dimethylethyl)phenyl]methyl}-4-hydroxy-6-oxo-2-(3-thienyl)-1,6-dihydro-5-pyrimidinyl]carbonyl}glycine

64a) 3-{[4-(1,1-Dimethylethyl)phenyl]methyl}-6-hydroxy-2-(3-thienyl)-4(3H)-pyrimidinone. Dimethylaluminium chloride (1.212 ml, 1.212 mmol) was added to a solution of 3-thiophenecarbonitrile (144 mg, 1.322 mmol) and 4-t-butylbenzylamine (0.194 ml, 1.102 mmol) in yoluene (1.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (4.0 ml). Diethylmalonate (0.668 ml, 4.41 mmol) and sodium methoxyde (1.008 ml, 4.41 mmol) were added and the mixture was stirred at reflux for 19 h. After cooling, the mixture was poured into water. The pH was adjusted to ca. 5 by the addition of 1 N HCl. The resulting precipitate was collected, washed with water and dried. It was then suspended in 3% MeOH in CHCl₃ and filtered through a 45 p PTFE (Teflon®) filter. The solvent was evaporated and the residue was purified on silica gel (0-8% MeOH in chloroform) to afford the title compound as an oil (100 mg, 0.294 mmol, 27.7% yield; 88% pure). The title compound was used as is in the next step. LCMS (ES) m/z 341 (MH^{|}).

64b) N-{[1-{[4-(1,1-Dimethylethyl)phenyl]methyl}-4-hydroxy-6-oxo-2-(3-thienyl)-1,6-dihydro-5-pyrimidinyl]carbonyl}glycine. A solution of 3-{[4-(1,1-dimethylethyl)phenyl]methyl}-6-hydroxy-2-(3-thienyl)-4(3H)-pyrimidinone (100 mg, 0.294 mmol), Hunig's base (0.131 mL, 0.735 mmol) and ethyl isocyanatoacetate (0.080 mL, 0.735 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (4.5 mL) and 1 M NaOH (4.5 mL, 4.5 mmol) and stirred at rt for 3.5 h. It was then puored into water and acidified by the addition of 1 N HCl. The precipitate was collected, washed with water and dried to give 115 mg of crude material. Purification by RP-HPLC (20 to 95% Acetonitrile in water, plus 0.1% TFA) afforded the title compound (50 mg, 0.113 mmol, 38.5% yield); tan powder. ¹H-NMR (400 MHz, CHLOROFORM-D) δ ppm 9.92 (t, J=5.43 Hz, 1 H), 7.72 (dd, J=2.78, 1.26 Hz, 1 H), 7.40 (dd, J=5.05, 3.03 Hz, 1 H), 7.37 (d, J=8.34 Hz, 2 H), 7.27-7.30 (m, 1 H), 6.99 (d, J=8.34 Hz, 2 H), 5.31 (br. s., 2 H), 4.25 (d, J=5.56 Hz, 2 H), 1.31 (s, 9 H). LCMS (ES^{|}) m/z 442 (MH^{|}).

### EXAMPLE 65

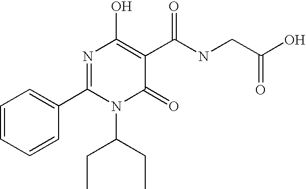

### N-{[1-(1-Ethylpropyl)-4-hydroxy-6-oxo-2-phenyl-1,6-dihydro-5-pyrimidinyl]carbonyl}glycine

65a) 3-(1-Ethylpropyl)-6-hydroxy-2-phenyl-4(3H)-pyrimidinone. Dimethylaluminium chloride (2.75 ml, 2.75 mmol) was added to a solution of 3-aminopentane (0.291 ml, 2.5 mmol) and benzonitrile (0.306 ml, 3.00 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.518 ml, 10.00 mmol) and sodium methoxide (2.288 ml, 10.00 mmol) were added and the mixture was stirred at reflux for 18 h. After cooling, the mixture was poured into water and the pH was adjusted to ca. 3 by the addition of 1 N HCl. The solid was collected, washed with water and dried to give 3-(1-ethylpropyl)-6-hydroxy-2-phenyl-4(3H)-pyrimidinone (456 mg, 1.765 mmol, 70.6% yield) as a white powder. ¹H-NMR (400 MHz, DMSO-d₆) δ ppm 11.45 (br. s., 1 H), 7.50-7.58 (m, 3 H), 7.42-7.48 (m, 2 H), 5.33 (s, 1 H), 3.61 (br. s., 1 H), 2.03-2.22 (m, 2 H), 1.70-1.86 (m, 2 H), 0.68 (t, J=7.58 Hz, 6 H). LCMS (ES^{|}) m/z 259 (MH^{|}).

65b) N-{[-(1-Ethylpropyl)-4-hydroxy-6-oxo-2-phenyl-1,6-dihydro-5-pyrimidinyl]carbonyl}glycine. A solution of 3-(1-ethylpropyl)-6-hydroxy-2-phenyl-4(3H)-pyrimidinone (350 mg, 1.355 mmol), Hunig's base (0.307 ml, 1.761 mmol) and ethyl isocyanatoacetate (0.198 ml, 1.761 mmol) in dichloromethane (DCM) (4 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (7.5 ml) and 1 M NaOH (7.5 ml, 7.50 mmol) and stirred at rt for 2 h. It was then poured into water and acidified by the addition of 1 N HCl. The precipitate was collected by filtration (410 mg, yellow powder, 88% pure by LC/MS). Recrystallization from EtOAc/Et2O (4:1) afforded N-{[1-(1-ethylpropyl)-4-hydroxy-6-oxo-2-phenyl-1,6-dihydro-5-pyrimidinyl]carbonyl}glycine (82 mg, 0.224 mmol, 16.50% yield). ¹H-NMR (400 MHz, DMSO-d₆) δ ppm 15.96 (s, 1 H), 12.91 (s, 1 H), 9.89 (t, J=5.56 Hz, 1 H), 7.56-7.62 (m, 3 H), 7.49-7.55 (m, 2 H), 4.10 (d, J=5.56 Hz, 2 H), 3.65-3.85 (m, 1 H), 2.06-2.23 (m, 2 H), 1.77-1.93 (m, 2 H), 0.70 (t, J=7.58 Hz, 6 H). LCMS (ES^{|}) m/z 360 (MH^{|}).

### EXAMPLE 66

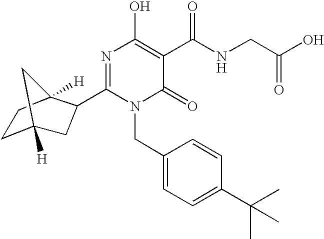

### N-[(2-[Bicyclo[2.2.1]hept-2-yl]-1-55 [4-(1,1-dimethylethyl)phenyl]methyl}-4-hydroxy-6-oxo-1,6-dihydro-5-pyrimidinyl)carbonyl]glycine

66a) 2-[Bicyclo[2.2.1]hept-2-yl]-3-{[4-(1,1-dimethylethyl)phenyl]methyl}-6-hydroxy-4(3H)-pyrimidinone. Dimethylaluminium chloride (1.212 ml, 1.212 mmol) was added to a solution of 2-norbonanecarbonitrile (mixture of endo and exo) (160 mg, 1.322 mmol) and 4-t-butylbenzylamine (0.194 ml, 1.102 mmol) in toluene (1.5 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (4.0 ml). Diethylmalonate (0.668 ml, 4.41 mmol) and sodium methoxide (1.008 ml, 4.41 mmol) were added and the mixture was stirred at reflux for 22 h. After cooling, the mixture was poured into water. The pH was adjusted to ca. 4 by the addition of 6 N HCl. The resulting precipitate was collected, washed with water and dried. It was then suspended in 3% MeOH in CHCl₃ and filtered through a 45μ PTFE filter. The solvent was evaporated and the residue was purified on silica gel (0-9% MeOH in chloroform) to afford the title compound as an oil (310 mg, 0.294 mmol, 79.8% yield; 85% pure). The title compound was used as is in the next step. LCMS (ES^{|}) m/z 353 (MH^{|}).

66b) N-[(2-Bicyclo[2.2.1]hept-2-yl-1-{[4-(1,1-dimethylethyl)phenyl]methyl}-4-hydroxy-4-oxo-1,6-dihydro-5-pyrimidinyl)carbonyl]glycine. A solution of 2-[bicyclo[2.2.1]hept-2-yl]-3-{[4-(1,1-dimethylethyl)phenyl]methyl}-6-hydroxy-4(3H)-pyrimidinone (300 mg, 0.85 mmol), Hunig's base (0.296 mL, 1.70 mmol) and ethyl isocyanatoacetate (0.190 mL, 1.70 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in EtOH (4.5 mL) and 1 M NaOH (4.5 mL, 4.5 mmol) and stirred at rt for 5 h. It was then puored into water and acidified by the addition of 1 N HCl. The precipitate was collected, washed with water and dried to give 180 mg of crude material. Purification by RP-HPLC (20 to 95% acetonitrile in water, plus 0.1% TFA) afforded the title compound (40 mg, 0.088 mmol, 10.4% yield); white powder. ¹H-NMR (400 MHz, CHLOROFORM-d) d ppm 9.97 (t, J=5.43 Hz, 1 H), 7.32-7.41 (m, 2 H), 6.98-7.09 (m, 2 H), 4.95-5.73 (m, 2 H), 4.08-4.33 (m, 2 H), 2.75 (dd, J=8.46, 5.43 Hz, 1 H), 1.98-2.57 (m, 3 H), 1.83 (d, J=9.85 Hz, 1 H), 1.41-1.71 (m, 3 H), 1.30 (s, 9 H), 1.14-1.26 (m, 3 H), LCMS (ES⁺) m/z 454 (MH⁻).

### EXAMPLE 67

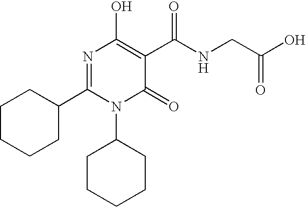

### N-[(1,2-Dicyclohexyl-4-hydroxy-6-oxo-1,6-dihydro-5-pyrimidinyl)carbonyl]glycine

67a) 2,3-Dicyclohexyl-6-hydroxy-4(3H)-pyrimidinone. Dimethylaluminium chloride (2.218 ml, 2.218 mmol) was added to a solution of cyclohexanecarbonitrile (0.287 ml, 2.420 mmol) and cyclohexylamine (0.231 ml, 2.017 mmol) in toluene (2.5 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (7 ml). Diethylmalonate (1.225 ml, 8.07 mmol) and sodium methoxyde (1.846 ml, 8.07 mmol) were added and the mixture was stirred at reflux for 19 h. After cooling, the mixture was poured into water, the pH was adjusted to 3-4 by the addition of 1 N HCl and extracted with EtOAc. The organics were washed with water, dried over Na₂SO₄ and evaporated. The residue was purified on silica gel (0-9% MeOH in chloroform) to afford 2,3-dicyclohexyl-6-hydroxy-4(3H)-pyrimidinone (335 mg, 1.212 mmol, 60.1% yield). LCMS (ES⁺) m/z 277 (MH⁺).

67b) N-[(1,2-Dicyclohexyl-4-hydroxy-6-oxo-1,6-dihydro-5-pyrimidinyl)carbonyl]glycine. A solution of 2,3-dicyclohexyl-6-hydroxy-4(3H)-pyrimidinone (327 mg, 1.183 mmol), Hunig's base (0.412 mL, 2.366 mmol) and ethyl isocyanatoacetate (0.265 mL, 2.366 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in and 1 M NaOH (7 mL, 7.00 mmol) and stirred at rt for 3 h. It was then puored into water and acidified by the addition of 1 N HCl. The precipitate was collected, washed with water and purified by RP-HPLC. The purified compound was recrystallized from toluene to afford N-[(1,2-dicyclohexyl-4-hydroxy-6-oxo-1,6-dihydro-5-pyrimidinyl)carbonyl]glycine (53 mg, 0.133 mmol, 11.27% yield); white powder. ¹H-NMR (400 MHz, CHLOROFORM-D) d ppm 15.13 (s, 1 H), 10.06 (t, J=5.18 Hz, 1 H), 4.24 (d, J=5.56 Hz, 2 H), 4.03 (s, 1 H), 2.56-2.92 (m, 2 H), 1.58-2.05 (m, 13 H), 1.07-1.53 (m, 6 H). LCMS (ES⁺) m/z 378 (MH⁺).

### EXAMPLE 68

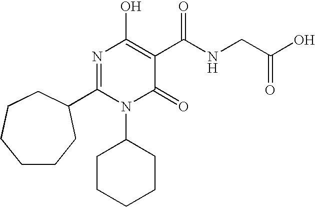

### N-[(2-Cycloheptyl-1-cyclohexyl-4-hydroxy-6-oxo-1,6-dihydro-5-pyrimidinyl)carbonyl]glycine

68a) 2-Cycloheptyl-3-cyclohexyl-6-hydroxy-4(3H)-pyrimidinone. Dimethylaluminium chloride (2.218 ml, 2.218 mmol) was added to a solution of cycloheptyl cyanide (0.269 ml, 2.017 mmol) and cyclohexylamine (0.231 ml, 2.017 mmol) in Toluene (2.5 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (7 ml). Diethylmalonate (1.225 ml, 8.07 mmol) and sodium methoxide (1.846 ml, 8.07 mmol) were added and the mixture was stirred at reflux for 20 h. After cooling, the mixture was poured into water, the pH was adjusted to 3-4 by the addition of 1 N HCl and extracted with EtOAc. The organics were washed with water, dried over Na₂SO₄ and evaporated. The residue was purified on silica gel (0-9% MeOH in chloroform) to afford 2-cycloheptyl-3-cyclohexyl-6-hydroxy-4(3H)-pyrimidinone (407 mg, 1.402 mmol, 69.5% yield). The title compound was used as is in the next step. LCMS (ES⁺) m/z 291 (MH⁺).

68b) N-[(2-Cycloheptyl-1-cyclohexyl-4-hydroxy-6-oxo-1,6-dihydro-5-pyrimidinyl)carbonyl]glycine. A solution of 2-cycloheptyl-3-cyclohexyl-6-hydroxy-4(3H)-pyrimidinone (400 mg, 1.377 mmol), Hunig's base (0.480 ml, 2.75 mmol) and ethyl isocyanatoacetate (0.309 ml, 2.75 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (6 mL) and 1 M NaOH (7.5 ml, 7.50 mmol) and stirred at rt for 3 h. It was then puored into water and acidified by the addition of 1 N HCl. The precipitate was collected, washed with water and dried. Recrystallyzation from DMSO/H₂O and then toluene afforded N-[(2-cycloheptyl-1-cyclohexyl-4-hydroxy-6-oxo-1,6-dihydro-5-pyrimidinyl)carbonyl]glycine (85 mg, 0.172 mmol, 12.48% yield) as a white powder as 1:1 DMSO solvate. ¹H-NMR (400 MHz, CHLOROFORM-D) δ ppm 15.22 (br. s., 1 H), 10.05 (br. s., 1 H), 4.22 (d, J=5.56 Hz, 2 H), 4.00 (br. s., 1 H), 2.69-2.80 (m, 1 H), 1.20-2.09 (m, 22 H). LCMS (ES⁺) m/z 392 (MH⁻).

### EXAMPLE 69

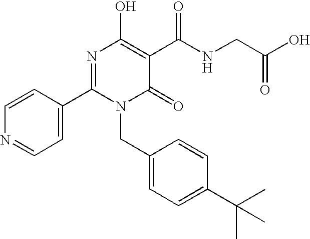

### N-{[1-{[4-(1,1-Dimethylethyl)phenyl]methyl}-4-hydroxy-6-oxo-2-(4-pyridinyl)-1,6-dihydro-5-pyrimidinyl]carbonyl}glycine

69a) 3-{[4-(1,1-Dimethylethyl)phenyl]methyl}-6-hydroxy-2-(4-pyridinyl)-4(3H)-pyrimidinone. Dimethylaluminium chloride (2.423 ml, 2.423 mmol) was added to a solution of 4-cyanopyridine (275 mg, 2.64 mmol) and 4-t-butylbenzylamine (0.388 ml, 2.203 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.338 ml, 8.81 mmol) and sodium methoxide (2.017 ml, 8.81 mmol) were added and the mixture was stirred at reflux for 18 h. After cooling, the mixture was poured into water. The pH was adjusted to ca. 3-4 by the addition of 1 N HCl and extracted with EtOAc. The organics were washed with brine, dried over Na₂SO₄ and evaporated. The residue was purified on silica gel (0-9% MeOH in chloroform) to afford 3-{[4-(1,1-dimethylethyl)phenyl]methyl}-6-hydroxy-2-(4-pyridinyl)-4(3H)-pyrimidinone (385 mg, 1.090 mmol, 49.5% yield). ¹H NMR (400 MHz, CHLOROFORM-d) d ppm 8.70 (d, J=4.80 Hz, 2 H), 7.22-7.27 (m, 2 H), 7.14 (d, J=5.81 Hz, 2 H), 6.76-6.83 (m, 2 H), 5.77 (s, 1 H), 5.07 (s, 2 H), 1.28 (s, 9 H). LCMS (ES⁺) m/z 336 (MH⁺).

69b) N-{[1-{[4-(1,1-Dimethylethyl)phenyl]methyl}-4-hydroxy-6-oxo-2-(4-pyridinyl)-1,6-dihydro-5-pyrimidinyl]carbonyl}glycine. A solution of 3-{[4-(1,1-dimethylethyl)phenyl]methyl}-6-hydroxy-2-(4-pyridinyl)-4(3H)-pyrimidinone (380 mg, 1.133 mmol), Hunig's base (0.395 ml, 2.266 mmol) and ethyl isocyanatoacetate (0.254 ml, 2.266 mmol) in dichloromethane (DCM) (3.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (6 mL) and 1 M NaOH (7 ml, 7.00 mmol) and stirred at rt for 3 h. It was then poured into water and acidified by the addition of 1 N HCl. The precipitate was collected, washed with water and dried (285 mg, 94% pure by LC/MS). 120 mg were purified by RP-HPLC (20-90% acetonitrile in water, plus 0.1% TFA). The fraction containing product were combined and the solvent volume was reduced. The precipitate obtained was collected, washed with water and dried to afford the title compound as a white solid (42 mg) as the TFA salt. ¹H-NMR (400 MHz, DMSO-d₆) d ppm 16.13 (s, 1 H), 12.92 (s, 1 H), 9.81 (t, J=5.56 Hz, 1 H), 8.68-8.73 (m, 2 H), 7.50-7.55 (m, 2 H), 7.27 (d, J=8.59 Hz, 2 H), 6.95 (d, J=8.34 Hz, 2 H), 5.03 (s, 2 H), 4.09 (d, J=5.56 Hz, 2 H), 1.23 (s, 9 H). LCMS (ES⁺) m/z 437 (MH⁺).

### EXAMPLE 70

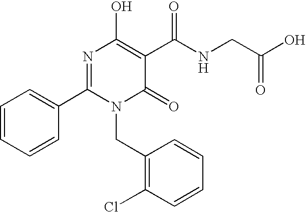

### N-({1-[(2-Chlorophenyl)methyl]-4-hydroxy-6-oxo-2-phenyl-1,6-dihydro-5-pyrimidinyl}carbonyl)glycine

70a) 3-[(2-Chlorophenyl)methyl]-6-hydroxy-2-phenyl-4(3H)-pyrimidinone. 1M Dimethylaluminium chloride in hexane (2.75 mL, 2.75 mmol) was added to a stirred solution of 2-chlorobenzylamine (0.354 g, 2.50 mmol) and benzonitrile (0.309 g, 3.00 mmol) in toluene (2 mL) and the mixture stirred for 15 min at room temperature under nitrogen, then microwaved at 150° C. for 45 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (350 mL). The mixture was washed with ether, then acidified to pH 1 with 6M aqueous hydrochloric acid and extracted again with ethyl acetate. The crude product was only partially soluble in ethyl acetate, so the organic solvent was removed under reduced pressure and the residue dissolved in 1M aqueous sodium hydroxide (20 mL). The pH was adjusted to 1 with 6M aqueous hydrochloric acid and the precipitate filtered, washed with water and dried. The solid was triturated with ether and dried, then chromatographed (silica gel, 3-10% methanol/dichloromethane) to give the title compound (0.127 g, 16%) as a solid. 1 H NMR (400 MHz, DMSO-d₆) δ ppm 5.02 (s, 2 H) 5.52 (s, 1 H) 6.96 (dd, J=7.33, 1.52 Hz, 1 H) 7.24-7.34 (m, 2 H) 7.35-7.42 (m, 5 H) 7.43-7.50 (m, 1 H) 11.77 (br. s., 1 H).

70b) N-({1-[(2-Chlorophenyl)methyl]-4-hydroxy-6-oxo-2-2-phenyl-1,6-dihydro-5-pyrimidinyl}carbonyl)glycine. A mixture of 3-[(2-chlorophenyl)methyl]-6-hydroxy-2-phenyl-4(3H)-pyrimidinone (0.125 g, 0.400 mmol), ethyl isocyanatoacetate (0.090 mL, 0.799 mmol), diisopropylethylamine (0.140 mL, 0.799 mmol) and dichloromethane (1.5 mL) was stirred in a microwave reactor at 140° C. for 1 h, then cooled. Trifluoroacetic acid (0.125 mL, 1.62 mmol) was added and the mixture chromatographed directly (silica gel, 1-10% methanol/dichloromethane) to give the intermediate ester, sufficiently pure for the next step. 1M aqueous sodium hydroxide (2.00 mL, 2.00 mmol) was added slowly to a stirred suspension of the intermediate ester in ethanol (6 mL) and the solution stirred for 2 h at room temperature, then diluted with water (30 mL) and acidified to pH 1 with 6M aqueous hydrochloric acid. The mixture was stirred 15 min, then the precipitated solid filtered, washed with water and dried to give the title compound (0.065 g, 39%) as a white solid. 1H NMR (400 MHz, DMSO-d₆) δ ppm 4.08 (d, J=5.56 Hz, 2 H) 5.08 (s, 2 H) 7.17-7.23 (m, 1 H) 7.27-7.34 (m, 2 H) 7.38-7.50 (m, 5 H) 7.49-7.57 (m, 1 H) 9.79 (t, J=5.56 Hz, 1 H) 12.93 (br. s., 1 H) 16.09 (br. s., 1 H).

### EXAMPLE 71

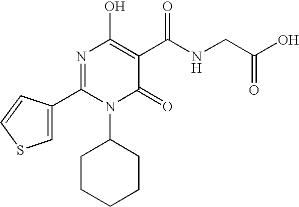

### N-{[1-Cyclohexyl-4-hydroxy-6-oxo-2-(3-thienyl)-1,6-dihydro-5-pyrimidinyl]carbonyl}glycine

71a) 3-Cyclohexyl-6-hydroxy-2-(3-thienyl)-4(3H)-pyrimidinone. Dimethylaluminium chloride (2.218 ml, 2.218 mmol) was added to a solution of 3-thiophenecarbonitrile (0.220 ml, 2.420 mmol) and cyclohexylamine (0.231 ml, 2.017 mmol) in Toluene (2.5 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.225 ml, 8.07 mmol) and sodium methoxide (1.846 ml, 8.07 mmol) were added and the mixture was stirred at reflux for 19 h. After cooling, the mixture was poured into water, the pH was adjusted to 3-4 by te addition of 1 N HCl and extracted with EtOAc. The organics were washed with water, dried over Na₂SO₄ and evaporated. The residue was purified on silica gel (ISCO, 0-9% MeOH in chloroform) to afford 3-cyclohexyl-6-hydroxy-2-(3-thienyl)-4(3H)-pyrimidinone (297 mg, 1.075 mmol, 53.3% yield) (yellow powder). ¹H NMR (400 MHz, DMSO-d₆) δ ppm 7.92 (dd, J=2.78, 1.26 Hz, 1 H), 7.74 (dd, J=4.93, 2.91 Hz, 1 H), 7.32 (dd, J=5.05, 1.26 Hz, 1 H), 5.29 (s, 1 H), 3.73-3.85 (m, 1 H), 2.42-2.57 (m, 2 H), 1.60-1.76 (m, 4 H), 1.44-1.55 (m, 1 H), 0.82-1.14 (m, 3 H). LCMS (ES⁺) m/z 277 (MH⁺).

71b) N-{[1-Cyclohexyl-4-hydroxy-6-oxo-2-(3-thienyl)-1,6-dihydro-5-pyrimidinyl]carbonyl}glycine. A solution of 3-cyclohexyl-6-hydroxy-2-(3-thienyl)-4(3H)-pyrimidinone (255 mg, 0.923 mmol), Hunig's base (0.321 ml, 1.845 mmol) and ethyl isocyanatoacetate (0.207 ml, 1.845 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (6 mL) and 1 M NaOH (7.5 ml, 7.50 mmol) and stirred at rt for 3 h. It was then poured into water and acidified by the addition of 1 N HCl. The precipitate was collected, washed with water and dried. Recrystallization from toluene provided N-{[1-cyclohexyl-4-hydroxy-6-oxo-2-(3-thienyl)-1,6-dihydro-5-pyrimidinyl]carbonyl}glycine (130 mg, 0.327 mmol, 35.5% yield); white solid. ¹H-NMR (400 MHz, DMSO-d₆) δ ppm 9.93 (t, J=5.31 Hz, 1 H), 8.03 (dd, J=3.03, 1.26 Hz, 1 H), 7.77 (dd, J=5.05, 3.03 Hz, 1 H), 7.37 (dd, J=5.05, 1.26 Hz, 1 H), 3.99 (d, J=5.31 Hz, 2 H), 3.86-3.99 (m, 1 H), 2.43-2.57 (m, 2 H), 1.67-1.88 (m, J=23.75, 11.87 Hz, 4 H), 1.54 (d, J=12.38 Hz, 1 H), 0.88-1.18 (m, 3 H). LCMS (ES⁺) m/z 378 (MH+).

### EXAMPLE 72

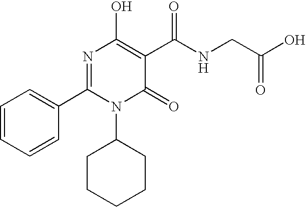

### N-[(1-Cyclohexyl-4-hydroxy-6-oxo-2-phenyl-1,6-dihydro-5-pyrimidinyl)carbonyl]glycine

72a) 3-Cyclohexyl-6-hydroxy-2-phenyl-4(3H)-pyrimidinone. 1 M Dimethylaluminium chloride in hexane (5.50 mL, 5.50 mmol) was injected into a stirred mixture of benzonitrile (0.691 g, 6.00 mmol), cyclohexylamine (0.496 g, 5.00 mmol) and toluene (4 mL) and the mixture stirred for 10 min at room temperature, and for 40 min at 150° C. in a microwave synthesiser, then cooled. The solvent was removed under reduced pressure and diethyl malonate (3.20 g, 20.0 mmol) added, followed by 2-methoxyethanol (15 mL) and 4.37 M methanolic sodium methoxide solution (4.58 ml, 20.0 mmol). The resulting mixture was refluxed under nitrogen for 18 h, then cooled and poured into water (200 mL). The mixture was washed with ether, acidified to pH 1 with 6M aqueous hydrochloric acid and extracted with ethyl acetate. The extracts were washed with water, brine, dried (MgSO₄) and evaporated under reduced pressure. The residue was triturated with ether and the solid collected, washed with ether and dried to give the title compound (0.658 g, 49%) as a white solid. 1H NMR (400 MHz, DMSO-d₆) δ ppm 0.70-0.90 (m, 2 H) 0.95-1.08 (m, 1 H) 1.40-1.52 (m, J=12.63 Hz, 1 H) 1.60-1.72 (m, 4 H) 2.43-2.57 (m, 2 H) 3.60-3.74 (m, J=11.75, 11.75 Hz, 1 H) 5.32 (s, 1 H) 7.49-7.61 (m, 5 H) 11.42 (br. s., 1 H).

72b) N-[(1-Cyclohexyl-4-hydroxy-6-oxo-2-phenyl-1,6-dihydro-5-pyrimidinyl)carbonyl]glycine. A mixture of 3-cyclohexyl-6-hydroxy-2-phenyl-4(3H)-pyrimidinone (0.657 g, 2.43 mmol), ethyl isocyanatoacetate (0.545 mL, 4.86 mmol), diisopropylethylamine (0.849 mL, 4.86 mmol) and dichloromethane (3 mL) was stirred in a microwave reactor at 140° C. for 1 h, then cooled. Trifluoroacetic acid (0.75 mL) was added and the mixture chromatographed directly (silica gel, 1-9% methanol/dichloromethane) to give the intermediate ester as a foam. 0.199 g of this material was stirred at room temperature in ethanol (15 mL) and 1 M aqueous sodium hydroxide (5.00 mL, 5.00 mmol) added slowly. The resulting solution was stirred at room temperature for 18 h, then diluted with water (50 mL) and acidified to pH 1 with 6M aqueous hydrochloric acid. The precipitate was filtered, washed with water and dried to give the title compound (0.153 g, 75%) as a white solid. 1H NMR (400 MHz, DMSO-d₆) δ ppm 0.74-0.92 (m, 2 H) 0.97-1.13 (m, 1 H) 1.42-1.54 (m, 1 H) 1.63-1.86 (m, 4 H) 2.41-2.49 (m, 2 H) 3.70-3.84 (m, 1 H) 4.09 (d, J=5.56 Hz, 2 H) 7.53-7.66 (m, 5 H) 9.95 (t, J=5.56 Hz, 1 H) 12.92 (br. s., 1 H) 15.94 (br. s., 1 H).

### EXAMPLE 73

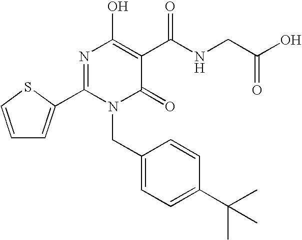

### N-{[1-{[4-(1,1-Dimethylethyl)phenyl]methyl}-4-hydroxy-6-oxo-2-(2-thienyl)-1,6-dihydro-5-pyrimidinyl]carbonyl}glycine

Dimethylaluminium chloride (2.426 mL, 2.426 mmol) was added to a solution of 2-thiophenecarbonitrile (0.246 mL, 2.65 mmol) and 4-t-butylbenzylamine (0.388 mL, 2.205 mmol) in toluene (2.8 mL). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 mL). Diethylmalonate (1.339 mL, 8.82 mmol) and sodium methoxide (2.018 mL, 8.82 mmol) were added and the mixture was stirred at reflux for 18 h. After cooling, the mixture was poured into water, acidified with 1N HCl (pH=3-4) and extracted with EtOAc. The organics were washed with brine, dried over Na₂SO₄ and evaporated. The residue was purified on silica gel (0-7.5% MeOH in CHCl₃), but poor separation from impurity (brown oil, 72% pure, not big improvement from crude, 850 mg). The residue was dissolved in dichloromethane (DCM) (3.5 mL) and ethyl isocyanatoacetate (0.495 mL, 4.41 mmol) and Hunig's base (0.768 mL, 4.41 mmol) were added. The mixture was irradiated at 130° C. for 1 h in a Biotage Initiator® microwave synthesizer, then diluted with dichloromethane and washed with 1 N HCl. The organic phase was dried over Na₂SO₄ and evaporated. The residue was dissolved in ethanol (6 mL) and 1 M NaOH (7 ml, 7.00 mmol) and stirred at rt for 4 h. It was then poured into water and extracted with EtOAc. The aqueous was acidified by the addition of 1 N HCl and extracted with EtOAc. The organic layer was washed with brine, dried over Na₂SO₄ and evaporated. Yellow oil, solidify on standing, 850 mg (85% pure); purified by RP-HPLC (20 to 95% acetonitrile in water, plus 0.1% TFA) to afford N-{[1-{[4-(1,1-dimethylethyl)phenyl]methyl}-4-hydroxy-6-oxo-2-(2-thienyl)-1,6-dihydro-5-pyrimidinyl]carbonyl}glycine (205 mg, 0.450 mmol, 20.43% yield). Yellow powder. ¹H-NMR (400 MHz, DMSO-d₆) d ppm 15.93 (s, 1 H), 12.92 (s, 1 H), 9.76 (t, J=5.68 Hz, 1 H), 8.00 (d, J=5.31 Hz, 1 H), 7.47 (dd,J=3.79, 1.01 Hz, 1 H), 7.38 (d, J=8.59 Hz, 2 H), 7.18 (dd, J=5.05, 4.04 Hz, 1 H), 7.13 (d, J=8.34 Hz, 2 H), 5.43 (s, 2 H), 4.06 (d, J=5.56 Hz, 2 H), 1.26 (s, 9 H). LCMS (ES⁺) m/z 442 (MH⁺).

### EXAMPLE 74

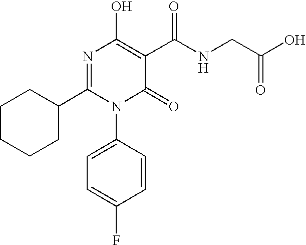

### N-{[2-Cyclohexyl-1-(4-fluorophenyl)-4-hydroxy-6-oxo-1,6-dihydro-5-pyrimidinyl]carbonyl}glycine

74a) 2-Cyclohexyl-3-(4-fluorophenyl)-6-hydroxy-4(3H)-pyrimidinone. 1M Dimethylaluminium chloride in hexane (2.75 mL, 2.75 mmol) was added to a stirred solution of 4-fluoroaniline (0.278 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) to give the title compound (0.252 g, 35%) as a cream solid. 1H NMR (400 MHz, DMSO-d₆) δ ppm 0.80-0.96 (m, 2 H) 1.03-1.17 (m, 1 H) 1.41-1.58 (m, 3 H) 1.59-1.79 (m, 4 H) 2.02-2.16 (m, 1 H) 5.31 (s, H) 11.38 (br. s., 1 H).

74b) N-{[2-Cyclohexyl-1-(4-fluorophenyl)-4-hydroxy-6-oxo-1,6-dihydro-5-pyrimidinyl]carbonyl}glycine. A mixture of 2-cyclohexyl-3-(4-fluorophenyl)-6-hydroxy-4(3H)-pyrimidinone (0.250 g, 0.867 mmol), ethyl isocyanatoacetate (0.243 mL, 2.17 mmol), diisopropylethylamine (0.379 mL, 2.17 mmol) and dichloromethane (3 mL) was stirred in a microwave reactor at 140° C. for 1 h, then cooled. Trifluoroacetic acid (0.334 mL, 4.34 mmol) was added and the mixture chromatographed directly (silica gel, 1-10% methanol/dichloromethane) to give the intermediate ester, sufficiently pure for the next step. 1M aqueous sodium hydroxide (5.00 mL, 5.00 mmol) was added slowly to a stirred suspension of the intermediate ester in ethanol (20 mL) and the solution stirred for 2 h at room temperature, then diluted with water (80 mL) and acidified to pH 1 with 6M aqueous hydrochloric acid. The mixture was stirred 15 min, then the precipitated solid filtered, washed with water and dried to give the title compound (0.180 g, 53%) as a white solid. 1H NMR (400 MHz, DMSO-d₆) δ ppm 0.80-0.97 (m, 2 H) 1.07-1.21 (m, 1 H) 1.41-1.58 (m, 3 H) 1.60-1.70 (m, 2 H) 1.72-1.83 (m, 2 H) 2.07-2.19 (m, 1 H) 4.04 (d, J=5.81 Hz, 2 H) 7.38-7.48 (m, 2 H) 7.53-7.63 (m, 2 H) 9.70 (t, J=5.56 Hz, 1 H) 12.90 (br. s., 1 H) 15.93 (s, 1 H).

### EXAMPLE 75

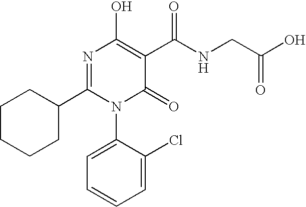

### N-{[1-(2-Chlorophenyl)-2-cyclohexyl-4-hydroxy-6-oxo-1,6-dihydro-5-pyrimidinyl]carbonyl}glycine

75a) 3-(2-Chlorophenyl)-2-cyclohexyl-6-hydroxy-4(3H)-pyrimidinone. 1M Dimethylaluminium chloride in hexane (2.75 mL, 2.75 mmol) was added to a stirred solution of 2-chloroaniline (0.319 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-10% methanol/dichloromethane) to give the title compound (0.230 g, 30%) as a white solid. 1H NMR (400 MHz, DMSO-d₆) δ ppm 0.76-0.99 (m, 2 H) 1.02-1.19 (m, 1 H) 1.33-1.46 (m, 1 H) 1.47-1.84 (m, 6 H) 1.89-2.02 (m, 1 H) 5.36 (s, 1 H) 7.47-7.61 (m, 3 H) 7.67-7.77 (m, 1 H) 11.52 (br. s., 1 H).

75b) N-{[1-(2-Chlorophenyl)-2-cyclohexyl-4-hydroxy-6-oxo-1,6-dihydro-5-pyrimidinyl]carbonyl}glycine. A mixture of 3-(2-chlorophenyl)-2-cyclohexyl-6-hydroxy-4(3H)-pyrimidinone (0.228 g, 0.748 mmol), ethyl isocyanatoacetate (0.210 mL, 1.87 mmol), diisopropylethylamine (0.327 mL, 1.87 mmol) and dichloromethane (3 mL) was stirred in a microwave reactor at 140° C. for 1 h, then cooled. Trifluoroacetic acid (0.288 mL, 3.74 mmol) was added and the mixture chromatographed directly (silica gel, 1-10% methanol/dichloromethane) to give the intermediate ester, sufficiently pure for the next step. 1M aqueous sodium hydroxide (5.00 mL, 5.00 mmol) was added slowly to a stirred suspension of the intermediate ester in ethanol (15 mL) and the solution stirred for 18 h at room temperature, then diluted with water (80 mL) and acidified to pH 1 with 6M aqueous hydrochloric acid. The mixture was stirred 15 min, then the precipitated solid filtered, washed with water and dried to give the title compound (0.157 g, 52%) as a white solid. 1H NMR (400 MHz, DMSO-d₆) δ ppm 0.74-1.01 (m, 2 H) 1.08-1.22 (m, 1 H) 1.34-1.75 (m, 6 H) 1.77-1.87 (m, 1 H) 1.94-2.08 (m, 1 H) 3.97-4.12 (m, 2 H) 7.54-7.67 (m, 2 H) 7.72-7.82 (m, 2 H) 9.62 (t, J=5.56 Hz, 1 H) 12.94 (br. s., 1 H) 16.12 (s, 1 H).

### EXAMPLE 76

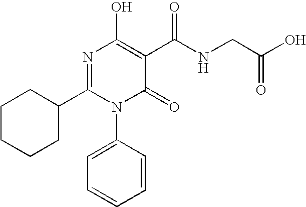

### N-[(2-Cyclohexyl-4-hydroxy-6-oxo-1-phenyl-1,6-dihydro-5-pyrimidinyl)carbonyl]glycine

76a) 2-Cyclohexyl-6-hydroxy-3-phenyl-4(3H)-pyrimidinone. 1M Dimethylaluminium chloride in hexane (2.75 mL, 2.75 mmol) was added to a stirred solution of aniline (0.233 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) to give the title compound (0.205 g, 30%) as a white solid. 1H NMR (400 MHz, DMSO-d₆) δ ppm 0.77-0.91 (m, 2 H) 1.04-1.17 (m, 1 H) 1.40-1.57 (m, 3 H) 1.58-1.80 (m, 4 H) 2.01-2.15 (m, 1 H) 5.31 (s, 1 H) 7.26-7.38 (m, 2 H) 7.44-7.62 (m, 3 H) 11.35 (br. s., 1 H).

76b) N-[(2-Cyclohexyl-4-hydroxy-6-oxo-1-phenyl-1,6-dihydro-5-pyrimidinyl)carbonyl]glycine. A mixture of 2-cyclohexyl-6-hydroxy-3-phenyl-4(3H)-pyrimidinone (0.203 g, 0.751 mmol), ethyl isocyanatoacetate (0.211 mL, 1.88 mmol), diisopropylethylamine (0.328 mL, 1.88 mmol) and dichloromethane (3 mL) was stirred in a microwave reactor at 140° C. for 1 h, then cooled. Trifluoroacetic acid (0.290 mL, 3.76 mmol) was added and the mixture chromatographed directly (silica gel, 1-10% methanol/dichloromethane) to give the intermediate ester, sufficiently pure for the next step. 1M aqueous sodium hydroxide (5.00 mL, 5.00 mmol) was added slowly to a stirred suspension of the intermediate ester in ethanol (20 mL) and the solution stirred for 2 h at room temperature, then diluted with water (80 mL) and acidified to pH 1 with 6M aqueous hydrochloric acid. The mixture was stirred 15 min, then the precipitated solid filtered, washed with water and dried to give the title compound (0.150 g, 54%) as a white solid. 1H NMR (400 MHz, DMSO-d₆) δ ppm 0.75-0.91 (m, 2 H) 1.05-1.21 (m, 1 H) 1.42-1.57 (m, 3 H) 1.60-1.70 (m, 2 H) 1.72-1.83 (m, 2 H) 2.08-2.18 (m, 1 H) 4.04 (d, J=5.56 Hz, 2 H) 7.44-7.52 (m, 2 H) 7.52-7.63 (m, 3 H) 9.71 (t, J=5.56 Hz, 1 H) 12.91 (br. s., 1 H) 15.92 (s, 1 H).

### EXAMPLE 77

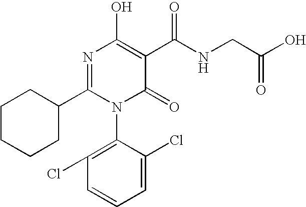

### N-{[2-Cyclohexyl-1-(2,6-dichlorophenyl)-4-hydroxy-6-oxo-1,6-dihydro-5-pyrimidinyl]carbonyl}glycine

77a) 2-Cyclohexyl-3-(2,6-dichlorophenyl)-6-hydroxy-4(3H)-pyrimidinone. 1M Dimethylaluminium chloride in hexane (2.75 mL, 2.75 mmol) was added to a stirred solution of 2,6-dichloroaniline (0.405 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-10% methanol/dichloromethane) to give the title compound (0.226 g, 27%) as a white solid. 1H NMR (400 MHz, DMSO-d₆) δ ppm 0.88-1.04 (m, 2 H) 1.05-1.17 (m, 1 H) 1.46-1.62 (m, 3 H) 1.63-1.82 (m, 4 H) 1.87-1.96 (m, 1 H) 5.41 (s, 1 H) 7.61 (dd, J=8.62, 7.58 Hz, 1 H) 7.72-7.81 (m, 2 H) 12.76 (br. s., 1 H).

77b) N-{[2-Cyclohexyl-1-(2,6-dichlorophenyl)-4-hydroxy-6-oxo-1,6-dihydro-5-pyrimidinyl]carbonyl}glycine. A mixture of 2-cyclohexyl-3-(2,6-dichlorophenyl)-6-hydroxy-4(3H)-pyrimidinone (0.224 g, 0.751 mmol), ethyl isocyanatoacetate (0.185 mL, 1.65 mmol), diisopropylethylamine (0.288 mL, 1.65 mmol) and dichloromethane (3 mL) was stirred in a microwave reactor at 140° C. for 1 h, then cooled. Trifluoroacetic acid (0.254 mL, 3.30 mmol) was added and the mixture chromatographed directly (silica gel, 1-10% methanol/dichloromethane) to give the intermediate ester, sufficiently pure for the next step. 1M aqueous sodium hydroxide (1.00 mL, 1.00 mmol) was added slowly to a stirred suspension of the intermediate ester in ethanol (5 mL) and the solution stirred for 18 h at room temperature, then diluted with water (30 mL) and acidified to pH 1 with 6M aqueous hydrochloric acid. The mixture was stirred 15 min, then the precipitated solid filtered, washed with water and dried. The solid was purified by reverse-phase preparative HPLC (ODS, 20-100% acetonitrile/water+0.1% trifluoroacetic acid) to give the title compound (0.027 g, 9%) as a solid. 1H NMR (400 MHz, DMSO-d₆) δ ppm 0.90-1.06 (m, 2 H) 1.12-1.25 (m, 1 H) 1.50-1.63 (m, 3 H) 1.66-1.84 (m, 4 H) 1.95-2.06 (m, 1 H) 4.06 (d, J=5.56 Hz, 2 H) 7.70 (dd, J=8.59, 7.58 Hz, 1 H) 7.81-7.88 (m, 2 H) 9.52 (t, J=5.43 Hz, 1 H) 12.97 (br. s., 1 H) 16.37 (s, 1 H).

### EXAMPLE 78

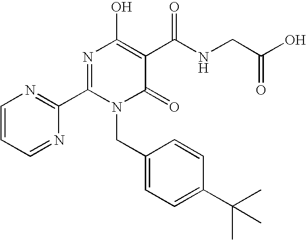

### N-[(1-{[4-(1,1-Dimethylethyl)phenyl]methyl}-4-hydroxy-6-oxo-1,6-dihydro-2,2′-bipyrimidin-5-yl)carbonyl[glycine

Dimethylaluminium chloride (2.423 ml, 2.423 mmol) was added to a suspension of 2-cyanopyrimidine (278 mg, 2.64 mmol) and 4-t-Butylbenzylamine (0.388 ml, 2.203 mmol) in Toluene (2.7 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.338 ml, 8.81 mmol) and sodium methoxyde (2.017 ml, 8.81 mmol) were added and the mixture was stirred at reflux for 18 h. After cooling, the mixture was poured into water. The pH was adjusted to ca. 3-4 by the addition of 1 N HCl and extracted with EtOAc. The organics were washed with brine, dried over Na₂SO₄ and evaporated. The residue was purified on silica gel (0-9% MeOH in chloroform) to afford 3-{[4-(1,1-dimethylethyl)phenyl]methyl}-6-hydroxy-5-methyl-2,2′-bipyrimidin-4(3H)-one (354 mg, red oil; LCMS (ES⁺) m/z 337 (MH⁺), 60% pure). The residue was dissolved in dichloromethane (DCM) (3.5 mL) and ethyl isocyanatoacetate (0.495 mL, 4.41 mmol) and Hunig's base (0.768 mL, 4.41 mmol) were added. The mixture was irradiated at 130° C. for 1 h in a Biotage Initiator® microwave synthesizer, then diluted with dichloromethane and washed with 1 N HCl. The organic phase was dried over Na₂SO₄ and evaporated. The residue was dissolved in ethanol (6 mL) and 1 M NaOH (7 ml, 7.00 mmol) and stirred at rt for 4 h. It was then poured into water and extracted with EtOAc. The aqueous was acidified by the addition of 1 N HCl and extracted with EtOAc. The organic layer was washed with brine, dried over Na₂SO₄ and evaporated. Purification by RP-HPLC afforded N-[(1-{[4-(1,1-dimethylethyl)phenyl]methyl}-4-hydroxy-6-oxo-1,6-dihydro-2,2′-bipyrimidin-5-yl)carbonyl]glycine (30.2 mg, 0.052 mmol, 2.361% yield) as an orange powder as the TFA salt. Yellow powder. ¹H-NMR (400 MHz, DMSO-d₆) δ ppm 16.22 (br. s., 1 H), 9.82 (t, J=5.56 Hz, 1 H), 8.97 (d, J=4.80 Hz, 2 H), 7.73 (t, J=4.93 Hz, 1 H), 7.24 (d, J=8.34 Hz, 2 H), 6.93 (d, J=8.34 Hz, 2 H), 5.15 (s, 2 H), 4.10 (d, J=5.56 Hz, 2 H), 1.21 (s, 9 H). LCMS (ES⁺) m/z 438 (MH⁺).

### EXAMPLE 79

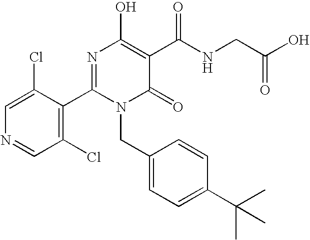

### N-[(2-(3,5-Dichloro-4-pyridinyl)-1-{[4-(1,1-dimethylethyl)phenyl]methyl}-4-dihydro-5-pyrimidinyl)carbonyl]glycine

79a) 2-(3,5-Dichloro-4-pyridinyl)-3-{[4-(1,1-dimethylethyl)phenyl]methyl}-6-hydroxy-4(3 H)-pyrimidinone. Dimethylaluminium chloride (2.423 ml, 2.423 mmol) was added to a solution of 3,5-dichloro-4-pyridinecarbonitrile (0.457 g, 2.64 mmol) and 4-t-butylbenzylamine (0.388 ml, 2.203 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.338 ml, 8.81 mmol) and sodium methoxide (2.017 ml, 8.81 mmol) were added and the mixture was stirred at reflux for 18 h. After cooling, the mixture was poured into water. The pH was adjusted to ca. 3-4 by te addition of 1 N HCl and extracted with EtOAc. The organics were washed with brine, dried over Na₂SO₄ and evaporated. The residue was purified on silica gel (0-9% MeOH in chloroform) to afford 2-(3,5-dichloro-4-pyridinyl)-3-{[4-(1,1-dimethylethyl)phenyl]methyl}-6-hydroxy-4(3H)-pyrimidinone (1.0 g, 2.152 mmol, 98% yield). Purity by LC/MS was 87%. The title compound was used as is in the next step. ¹H NMR (400 MHz, CHLOROFORM-D) δ ppm 8.55 (s, 2 H), 7.16 (ABq, JAB=8.59 Hz, 2 H), 6.72 (ABq, JAB=8.34 Hz, 2 H), 5.77 (s, 1 H), 4.99 (s, 2 H), 1.26 (s, 9 H). LCMS (ES+) m/z 404 (MH⁺).

79b) N-[(2-(3,5-Dichloro-4-pyridinyl)-1-{[4-(1,1-dimethylethyl)phenyl]methyl]-4-hydroxy-6-oxo-1,6-dihydro-5-pyrimidinyl)carbonyl]glycine. A solution of 2-(3,5-dichloro-4-pyridinyl)-3-{[4-(1,1-dimethylethyl)phenyl]methyl}-6-hydroxy-4(3H)-pyrimidinone (1 g, 2.473 mmol), Hunig's base (0.646 ml, 3.71 mmol) and ethyl isocyanatoacetate (0.416 ml, 3.71 mmol) in Dichloromethane (DCM) (4.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 mL) and 1 M NaOH (12 ml, 12.00 mmol) and stirred at rt for 3 h. It was then poured into water and acidified by the addition of 1 N HCl. The precipitate was collected, washed with water and dried (650 mg, ca. 90% pure by LC/MS). Various recrystallization attempts failed (solid is soluble in a variety of solvents). 500 mg were purified by RP-HPLC (25-95% acetonitrile in water plus 0.1% TFA) to afford N-[(2-(3,5-dichloro-4-pyridinyl)-1-{[4-(1,1-dimethylethyl)phenyl]methyl}-4-hydroxy-6-oxo-1,6-dihydro-5-pyrimidinyl)carbonyl]glycine (290 mg, 0.445 mmol, 17.98% yield). ¹H-NMR (400 MHz, DMSO-d₆) δ ppm 9.84 (t, J=5.68 Hz, 1 H), 8.82 (s, 2 H), 7.24 (d, J=8.59 Hz, 2 H), 6.87 (d, J=8.59 Hz, 2 H), 5.01 (s, 2 H), 4.13 (d, J=5.56 Hz, 2 H), 1.23 (s, 9 H). LCMS (ES⁻) m/z 505 (MH⁺).

### EXAMPLE 80

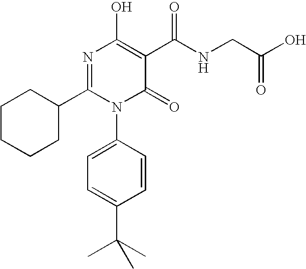

### N-({2-Cyclohexyl-1-[4-(1,1-dimethylethyl)phenyl]-4-hydroxy-6-oxo-1,6-dihydro-5-pyrimidinyl}carbonyl)glycine

80a) 2-cyclohexyl-3-[4-(1,1-dimethylethyl)phenyl]-6-hydroxy-4(3H)-pyrimidinone. 1 M Dimethylaluminium chloride in hexane (2.75 mL, 2.75 mmol) was added to a stirred solution of 4-t-butylaniline (0.373 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) to give the title compound (0.253 g, 31%) as a white solid. 1H NMR (400 MHz, DMSO-d₆) δ ppm 0.73-0.90 (m, 2 H) 1.04-1.16 (m, 1 H) 1.34 (s, 9 H) 1.41-1.57 (m, 3 H) 1.58-1.78 (m, 4 H) 2.05-2.16 (m, 1 H) 5.30 (s, 1 H) 7.22 (d, J=8.59 Hz, 2 H) 7.54 (d, J=8.59 Hz, 2 H) 11.32 (br. s., 1 H).

80b) N-({2-Cyclohexyl-1-[4-(1,1-dimethylethyl)phenyl]-4-hydroxy-6-oxo-1,6-dihydro-5-pyrimidinyl}carbonyl)glycine. A mixture of 2-cyclohexyl-3-[4-(1,1-dimethylethyl)phenyl]-6-hydroxy-4(3H)-pyrimidinone (0.215 g, 0.659 mmol), ethyl isocyanatoacetate (0.148 mL, 1.32 mmol), diisopropylethylamine (0.230 mL, 1.32 mmol) and dichloromethane (2 mL) was stirred in a microwave reactor at 140° C. for 1 h, then cooled. Trifluoroacetic acid (0.203 mL, 2.64 mmol) was added and the mixture chromatographed directly (silica gel, 1-9% methanol/dichloromethane) to give the intermediate ester, sufficiently pure for the next step. 1M aqueous sodium hydroxide (4.00 mL, 4.00 mmol) was added slowly to a stirred suspension of the intermediate ester in ethanol (20 mL) and the solution stirred for 2 h at room temperature, then diluted with water (80 mL) and acidified to pH 1 with 6M aqueous hydrochloric acid. The mixture was stirred 15 min, then the precipitated solid filtered, washed with water and dried to give the title compound (0.160 g, 57%) as a white solid. 1H NMR (400 MHz, DMSO-d₆) δ ppm 0.75-0.92 (m, 2 H) 1.05-1.21 (m, 1 H) 1.35 (s, 9 H) 1.41-1.58 (m, 3 H) 1.58-1.82 (m, 4 H) 2.16 (tt, J=11.37, 3.03 Hz, 1 H) 4.05 (d, J=5.56 Hz, 2 H) 7.34-7.42 (m, 2 H) 7.55-7.62 (m, 2 H) 9.73 (t, J=5.56 Hz, 1 H) 12.90 (br. s., 1 H) 15.87 (s, 1 H).

### EXAMPLE 81

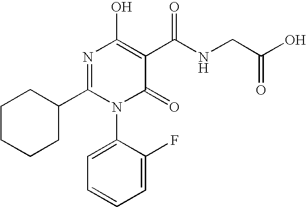

### N-{[2-Cyclohexyl-1-(2-fluorophenyl)-4-hydroxy-6-oxo-1,6-dihydro-5-pyrimidinyl]carbonyl}glycine

81a) 2-Cyclohexyl-3-(2-fluorophenyl)-6-hydroxy-4(3H)-pyrimidinone. 1M Dimethylaluminium chloride in hexane (2.75 mL, 2.75 mmol) was added to a stirred solution of 2-fluoroaniline (0.278 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and then triturated with ether. The solid was collected, washed with ether and dried to give the title compound (0.194 g, 27%) as a white solid. 1H NMR (400 MHz, DMSO-d₆) δ ppm 0.81-0.96 (m, 2 H) 1.04-1.17 (m, 1 H) 1.41-1.72 (m, 6 H) 1.75-1.85 (m, 1 H) 2.11 (tt, J=11.50, 3.10 Hz, 1 H) 5.35 (s, 1 H) 7.35-7.41 (m, 1 H) 7.43-7.50 (m, 1 H) 7.51-7.63 (m, 2 H) 11.56 (br. s., 1 H).

81b) N-{[2-Cyclohexyl-1-(2-fluorophenyl)-4-hydroxy-6-oxo-1,6-dihydro-5-pyrimidinyl]carbonyl}glycine. A mixture of 2-cyclohexyl-3-(2-fluorophenyl)-6-hydroxy-4(3H)-pyrimidinone (0.192 g, 0.666 mmol), ethyl isocyanatoacetate (0.149 mL, 1.33 mmol), diisopropylethylamine (0.233 mL, 1.33 mmol) and dichloromethane (2 mL) was stirred in a microwave reactor at 140° C. for 1 h, then cooled. Trifluoroacetic acid (0.203 mL, 2.64 mmol) was added and the mixture chromatographed directly (silica gel, 1-9% methanol/dichloromethane) to give the intermediate ester, sufficiently pure for the next step. 1M aqueous sodium hydroxide (4.00 mL, 4.00 mmol) was added slowly to a stirred suspension of the intermediate ester in ethanol (20 mL) and the solution stirred for 2 h at room temperature, then diluted with water (80 mL) and acidified to pH 1 with 6M aqueous hydrochloric acid. The mixture was stirred 15 min, then the precipitated solid filtered, washed with water and dried to give the title compound (0.155 g, 60%) as a white solid. 1H NMR (400 MHz, DMSO-d₆) δ ppm 0.80-1.01 (m, 2 H) 1.08-1.21 (m, 1 H) 1.42-1.74 (m, 6 H) 1.79-1.89 (m, 1 H) 2.11-2.23 (m, 1 H) 4.05 (d, J=5.56 Hz, 2 H) 7.39-7.48 (m, 1 H) 7.49-7.58 (m, 1 H) 7.61-7.74 (m, 2 H) 9.61 (t, J=5.56 Hz, 1 H) 12.93 (br. s., 1 H) 16.12 (s, 1 H).

### EXAMPLE 82

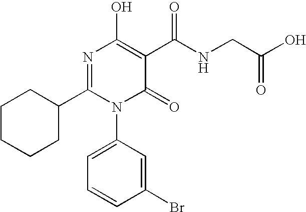

### N-{[1-(3-Bromophenyl)-2-cyclohexyl-4-hydroxy-6-oxo-1,6-dihydro-5-pyrimidinyl]carbonyl}glycine

82a) 3-(3-Bromophenyl)-2-cyclohexyl-6-hydroxy-4(3H)-pyrimidinone. 1M Dimethylaluminium chloride in hexane (2.75 mL, 2.75 mmol) was added to a stirred solution of 3-bromoaniline (0.430 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) to give the title compound (0.346 g, 40%) as a gum. 1H NMR (400 MHz, DMSO-d₆) δ ppm 0.77-0.99 (m, 2 H) 1.05-1.17 (m, 1 H) 1.37-1.58 (m, 3 H) 1.60-1.70 (m, 2 H) 1.71-1.80 (m, 2 H) 2.01-2.13 (m, 1 H) 5.31 (s, 1 H) 7.36-7.44 (m, 1 H) 7.50 (t, J=8.08 Hz, 1 H) 7.68-7.74 (m, 2 H) 11.46 (br. s., 1 H).

82b) N-{[1-(3-Bromophenyl)-2-cyclohexyl-4-hydroxy-6-oxo-1,6-dihydro-5-pyrimidinyl]carbonyl}glycine. A mixture of 3-(3-bromophenyl)-2-cyclohexyl-6-hydroxy-4(3H)-pyrimidinone (0.344 g, 0.985 mmol), ethyl isocyanatoacetate (0.331 mL, 2.96 mmol), diisopropylethylamine (0.516 mL, 2.96 mmol) and dichloromethane (3 mL) was stirred in a microwave reactor at 140° C. for 1 h, then cooled. Trifluoroacetic acid (0.456 mL, 5.92 mmol) was added and the mixture chromatographed directly (silica gel, 1-9% methanol/dichloromethane) to give the intermediate ester, sufficiently pure for the next step. 1M aqueous sodium hydroxide (6.00 mL, 6.00 mmol) was added slowly to a stirred suspension of the intermediate ester in ethanol (25 mL) and the solution stirred for 2 h at room temperature, then diluted with water (100 mL) and acidified to pH 1 with 6M aqueous hydrochloric acid. The mixture was stirred 15 min, then the precipitated solid filtered, washed with water and dried to give the title compound (0.202 g, 46%) as a white solid. 1H NMR (400 MHz, DMSO-d₆) δ ppm 0.80-0.98 (m, 2 H) 1.08-1.22 (m, 1 H) 1.42-1.59 (m, 3 H) 1.61-1.72 (m, 2 H) 1.74-1.83 (m, 2 H) 2.11 (tt, J=11.49, 3.16 Hz, 1 H) 4.05 (d, J=5.56 Hz, 2 H) 7.50-7.62 (m, 2 H) 7.73-7.81 (m, 1 H) 7.85-7.89 (m, 1 H) 9.66 (t, J=5.68 Hz, 1 H) 12.89 (br. s., 1 H) 15.94 (s, 1 H).

### EXAMPLE 83

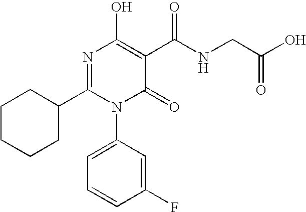

### N-{[2-Cyclohexyl-1-(3-fluorophenyl)-4-hydroxy-6-oxo-1,6-dihydro-5-pyrimidinyl]carbonyl}glycine

83a) 2-Cyclohexyl-3-(3-fluorophenyl)-6-hydroxy-4(3H)-pyrimidinone. 1M Dimethylaluminium chloride in hexane (2.75 mL, 2.75 mmol) was added to a stirred solution of 3-fluoroaniline (0.278 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) to give the title compound (0.327 g, 45%) as a gum. 1H NMR (400 MHz, DMSO-d₆) δ ppm 0.79-0.98 (m, 2 H) 1.04-1.16 (m, 1 H) 1.45-1.58 (m, 3 H) 1.59-1.83 (m, 4 H) 2.09 (tt, J=11.46, 3.19 Hz, 1 H) 5.32 (s, 1 H) 7.18-7.25 (m, 1 H) 7.33-7.45 (m, 2 H) 7.52-7.64 (m, 1 H) 11.48 (br. s., 1 H).

83b) N-{[2-Cyclohexyl-1-(3-fluorophenyl)-4-hydroxy-6-oxo-1,6-dihydro-5-pyrimidinyl]carbonyl}glycine. A mixture of 2-cyclohexyl-3-(3-fluorophenyl)-6-hydroxy-4(3H)-pyrimidinone (0.325 g, 1.13 mmol), ethyl isocyanatoacetate (0.379 mL, 3.38 mmol), diisopropylethylamine (0.591 mL, 3.38 mmol) and dichloromethane (3 mL) was stirred in a microwave reactor at 140° C. for 1 h, then cooled. Trifluoroacetic acid (0.521 mL, 6.76 mmol) was added and the mixture chromatographed directly (silica gel, 1-9% methanol/dichloromethane) to give the intermediate ester, sufficiently pure for the next step. 1M aqueous sodium hydroxide (6.00 mL, 6.00 mmol) was added slowly to a stirred suspension of the intermediate ester in ethanol (25 mL) and the solution stirred for 2 h at room temperature, then diluted with water (100 mL) and acidified to pH 1 with 6M aqueous hydrochloric acid. The mixture was stirred 15 min, then the precipitated solid filtered, washed with water and dried to give the title compound (0.199 g, 45%) as a white solid. 1H NMR (400 MHz, DMSO-d₆) δ ppm 0.80-0.98 (m, 2 H) 1.08-1.21 (m, 1 H) 1.42-1.59 (m, 3 H) 1.61-1.71 (m, 2 H) 1.74-1.84 (m, 2 H) 2.13 (tt, J=11.49, 3.16 Hz, 1 H) 4.05 (d, J=5.56 Hz, 2 H) 7.35-7.47 (m, 2 H) 7.51-7.58 (m, 1 H) 7.59-7.68 (m, 1 H) 9.67 (t, J=5.56 Hz, 1 H) 12.90 (br. s., 1 H) 15.96 (s, 1 H).

### EXAMPLE 84

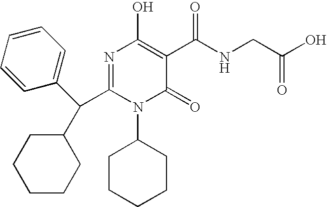

### N-({1-Cyclohexyl-2-[cyclohexyl(phenyl)methyl]-4-hydroxy-6-oxo-1,6-dihydro-5-pyrimidinyl}carbonyl)glycine

84a) 3-Cyclohexyl-2-[cyclohexyl(phenyl)methyl]-6-hydroxy-4(3H)-pyrimidinone. Dimethylaluminium chloride (2.77 ml, 2.77 mmol) was added to a solution of cyclohexyl(phenyl)acetonitrile (603 mg, 3.02 mmol) and Cyclohexylamine (0.288 ml, 2.52 mmol) in Toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.531 ml, 10.08 mmol) and sodium methoxide (2.307 ml, 10.08 mmol) were added and the mixture was stirred at reflux for 19 h. After cooling, the mixture was poured into water. The pH was adjusted to ca. 3-4 by te addition of 1 N HCl and extracted with EtOAc. The organics were washed with brine, dried over Na₂SO₄ and evaporated. The residue was purified on silica gel (0-8% MeOH in chloroform) to afford 3-cyclohexyl-2-[cyclohexyl(phenyl)methyl]-6-hydroxy-4(3H)-pyrimidinone (249 mg, 0.544 mmol, 21.56% yield; 80% pure by LC/MS). The title compound was used as is in the next step. LCMS (ES⁺) m/z 367 (MH⁻).

84b) N-({1-Cyclohexyl-2-[cyclohexyl(phenyl)methyl]-4-hydroxy-6-oxo-1,6-dihydro-5-pyrimidinyl}carbonyl)glycine. A solution of 3-cyclohexyl-2-[cyclohexyl(phenyl)methyl]-6-hydroxy-4(3H)-pyrimidinone (249 mg, 0.679 mmol), Hunig's base (0.237 ml, 1.359 mmol) and ethyl isocyanatoacetate (0.152 ml, 1.359 mmol) in dichloromethane (DCM) (2.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (5 mL) and 1 M NaOH (5 ml, 5.00 mmol) and stirred at rt for 3 h. It was then poured into water and acidified by the addition of 1 N HCl. The precipitate was collected and purified by RP-HPLC (50-100% acetonitrile in water, plus 0.1% TFA) to afford N-({1-cyclohexyl-2-[cyclohexyl(phenyl)methyl]-4-hydroxy-6-oxo-1,6-dihydro-5-pyrimidinyl}carbonyl)glycine (51 mg, 0.104 mmol, 15.25% yield) as a yellow powder. ¹H-NMR (400 MHz, DMSO-d₆) δ ppm 15.82 (s, 1 H), 12.85 (br. s., 1 H), 9.81 (t, J=5.68 Hz, 1 H), 7.18-7.46 (m, 5 H), 4.40 (t, J=11.75 Hz, 1 H), 4.11 (d, J=9.60 Hz, 1 H), 4.04 (d, J=5.81 Hz, 2 H), 2.17-2.32 (m, 2 H), 1.45-1.90 (m, 9 H), 0.87-1.32 (m, 9 H), 0.39-0.56 (m, 1 H). LCMS (ES⁺) m/z 468 (MH⁺).

### EXAMPLE 85

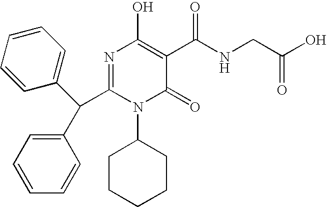

### N-{[1-Cyclohexyl-2-(diphenylmethyl)-4-hydroxy-6-oxo-1,6-dihydro-5-pyrimidinyl]carbonyl}glycine

85a) 3-Cyclohexyl-2-(diphenylmethyl)-6-hydroxy-4(3H)-pyrimidinone. Dimethylaluminium chloride (2.77 ml, 2.77 mmol) was added to a solution of diphenylacetonitrile (585 mg, 3.02 mmol) and cyclohexylamine (0.288 ml, 2.52 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.531 ml, 10.08 mmol) and sodium methoxide (2.307 ml, 10.08 mmol) were added and the mixture was stirred at reflux for 19 h. After cooling, the mixture was poured into water. The pH was adjusted to ca. 3 by the addition of 1 N HCl and extracted with EtOAc. The organics were washed with brine, dried over Na₂SO₄ and evaporated. The residue was purified on silica gel (0-8% MeOH in chloroform) to afford 3-cyclohexyl-2-(diphenylmethyl)-6-hydroxy-4(3H)-pyrimidinone (764 mg, 1.908 mmol, 76% yield; 90% pure by LC/MS). The title compound was used as is in the next step. LCMS (ES⁺) m/z 361 (MH⁺).

85b) N-{[1-Cyclohexyl-2-(diphenylmethyl)-4-hydroxy-6-oxo-1,6-dihydro-5-pyrimidinyl]carbonyl}glycine. A solution of 3-cyclohexyl-2-(diphenylmethyl)-6-hydroxy-4(3H)-pyrimidinone (764 mg, 2.12 mmol), Hunig's base (0.554 ml, 3.18 mmol) and ethyl isocyanatoacetate (0.357 ml, 3.18 mmol) in dichloromethane (DCM) (4 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 mL) and 1 M NaOH (10 ml, 10.00 mmol) and stirred at rt for 3 h. It was then poured into water and acidified by the addition of 1 N HCl. The precipitate was collected and purified by RP-HPLC (20-95% acetonitrile in water plus 0.1% TFA) to afford N-{[1-cyclohexyl-2-(diphenylmethyl)-4-hydroxy-6-oxo-1,6-dihydro-5-pyrimidinyl]carbonyl}glycine (200 mg, 0.412 mmol, 19.42% yield) as a yellow powder. ¹H-NMR (400 MHz, DMSO-d₆) δ ppm 15.73 (s, 1 H), 12.86 (br. s., 1 H), 9.85 (t, J=5.68 Hz, 1 H), 7.32-7.40 (m, 4 H), 7.20-7.32 (m, 6 H), 6.08 (s, 1 H), 4.22-4.37 (m, 1 H), 4.05 (d, J=5.81 Hz, 2 H), 2.28-2.44 (m, 2 H), 1.43-1.67 (m, 3 H), 1.16-1.28 (m, 2 H), 0.97-1.10 (m, 3 H). LCMS (ES⁺) m/z 462 (MH⁺).

### EXAMPLE 86

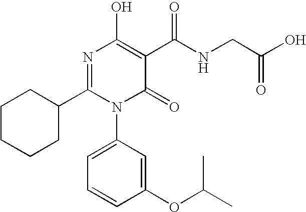

### N-[(2-Cyclohexyl-4-hydroxy-1-{3-[(1-methylethyl)oxy]phenyl}-6-oxo-1,6-dihydro-5-pyrimidinyl)carbonyl]glycine

86a) 2-Cyclohexyl-6-hydroxy-3-{3-[(1-methylethyl)oxy]phenyl}-4(3H)-pyrimidinone. 1M Dimethylaluminium chloride in hexane (2.75 mL, 2.75 mmol) was added to a stirred solution of 3-isopropoxyaniline (0.378 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.117 g, 14%) as a white solid. 1H NMR (400 MHz, DMSO-d₆) δ ppm 0.78-0.94 (m, 2 H) 1.04-1.17 (m, 1 H) 1.24 (d, J=6.06 Hz, 3 H) 1.28 (d, J=6.06 Hz, 3 H) 1.41-1.59 (m, 3 H) 1.59-1.84 (m, 4 H) 2.16 (tt, J=11.53, 3.16 Hz, 1 H) 4.66 (sept, J=5.98 Hz, 1 H) 5.30 (s, 1 H) 6.78-6.86 (m, 1 H) 6.91-6.96 (m, 1 H) 6.98-7.05 (m, 1 H) 7.40 (t, J=8.08 Hz, 1 H) 11.31 (br. s., 1 H).

86b) N-[(2-Cyclohexyl-4-hydroxy-1-{3-[(1-methylethyl)oxy]phenyl}-6-oxo-1,6-dihydro-5-pyrimidinyl)carbonyl]glycine. A mixture of 2-cyclohexyl-6-hydroxy-3-{3-[(1-methylethyl)oxy]phenyl}-4(3H)-pyrimidinone (0.115 g, 0.350 mmol), ethyl isocyanatoacetate (0.079 mL, 0.700 mmol), diisopropylethylamine (0.122 mL, 0.700 mmol) and dichloromethane (1 mL) was stirred in a microwave reactor at 140° C. for 1 h, then cooled. Trifluoroacetic acid (0.108 mL, 1.40 mmol) was added and the mixture chromatographed directly (silica gel, 1-9% methanol/dichloromethane) to give the intermediate ester, sufficiently pure for the next step. 1M aqueous sodium hydroxide (3.00 mL, 3.00 mmol) was added slowly to a stirred solution of the intermediate ester in ethanol (15 mL) and the solution stirred for 2 h at room temperature, then diluted with water (80 mL) and acidified to pH 1 with 6M aqueous hydrochloric acid. The mixture was stirred 1 h, then the precipitated solid filtered, washed with water and dried to give the title compound (0.087 g, 58%) as a white solid. 1H NMR (400 MHz, DMSO-d₆) δ ppm 0.78-0.95 (m, 2 H) 1.07-1.21 (m, 1 H) 1.25 (d, J=6.06 Hz, 3 H) 1.29 (d, J=5.81 Hz, 3 H) 1.43-1.58 (m, 3 H) 1.61-1.71 (m, 2 H) 1.72-1.87 (m, 2 H) 2.19 (tt, J=11.49, 3.16 Hz, 1 H) 4.05 (d, J=5.56 Hz, 2 H) 4.64 (sept, J=5.94 Hz, 1 H) 6.96-7.01 (m, 1 H) 7.05-7.10 (m, 1 H) 7.12-7.16 (m, 1 H) 7.44 (t, J=8.08 Hz, 1 H) 9.72 (t, J=5.56 Hz, 1 H) 12.90 (br. s., 1 H) 15.90 (s, 1 H).

### EXAMPLE 87

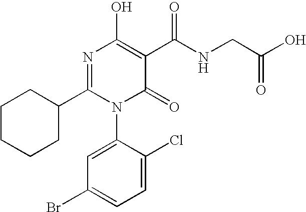

### N-{[1-(5-Bromo-2-chlorophenyl)-2-cyclohexyl-4-hydroxy-6-oxo-1,6-dihydro-5-pyrimidinyl]carbonyl}glycine

87a) 3-(5-Bromo-2-chlorophenyl)-2-cyclohexyl-6-hydroxy-4(3H)-pyrimidinone. 1M Dimethylaluminium chloride in hexane (2.75 mL, 2.75 mmol) was added to a stirred solution of 5-bromo-2-chloroaniline (0.516 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.270 g, 28%) as a white solid. 1H NMR (400 MHz, DMSO-d₆) δ ppm 0.82-1.03 (m, 2 H) 1.04-1.20 (m, 1 H) 1.31-1.47 (m, 1 H) 1.50-1.74 (m, 5 H) 1.77-1.88 (m, 1 H) 1.90-2.01 (m, 1 H) 5.35 (s, 1 H) 7.69 (d, J=8.59 Hz, 1 H) 7.78 (dd, J=8.70, 2.40 Hz, 1 H) 8.00 (d, J=2.27 Hz, 1 H) 11.60 (br. s., 1 H).

87b) N-{[1-(5-Bromo-2-chlorophenyl)-2-cyclohexyl-4-hydroxy-6-oxo-1,6-dihydro-5-pyrimidinyl]carbonyl}glycine. A mixture of 3-(5-bromo-2-chlorophenyl)-2-cyclohexyl-6-hydroxy-4(3H)-pyrimidinone (0.268 g, 0.699 mmol), ethyl isocyanatoacetate (0.157 mL, 1.38 mmol), diisopropylethylamine (0.244 mL, 1.38 mmol) and dichloromethane (2 mL) was stirred in a microwave reactor at 140° C. for 1 h, then cooled. Trifluoroacetic acid (0.213 mL, 2.76 mmol) was added and the mixture chromatographed directly (silica gel, 1-9% methanol/dichloromethane) to give the intermediate ester, sufficiently pure for the next step. 1M aqueous sodium hydroxide (5.00 mL, 5.00 mmol) was added slowly to a stirred solution of the intermediate ester in ethanol (20 mL) and the solution stirred for 18 h at room temperature, then diluted with water (100 mL) and acidified to pH 1 with 6M aqueous hydrochloric acid. The mixture was stirred 1 h, then the precipitated solid filtered, washed with water and dried to give the title compound (0.232 g, 69%) as a white solid. 1H NMR (400 MHz, DMSO-d₆) δ ppm 0.83-1.05 (m, 2 H) 1.10-1.22 (m, 1 H) 1.36-1.49 (m, 1 H) 1.52-1.76 (m, 5 H) 1.79-1.88 (m, 1 H) 1.98-2.07 (m, 1 H) 4.02 (dd, J=17.94, 5.56 Hz, 1 H) 4.07 (dd, J=17.94, 5.56 Hz, 1 H) 7.75 (d, J=8.59 Hz, 1 H) 7.85 (dd, J=8.72, 2.40 Hz, 1 H) 8.15 (d, J=2.53 Hz, 1 H) 9.57 (t, J=5.56 Hz, 1 H) 12.94 (br. s., 1 H) 16.17 (s, 1 H).

### EXAMPLE 88

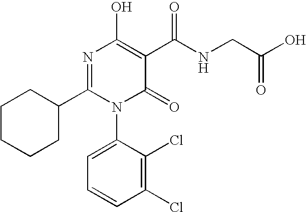

### N-{[2-Cyclohexyl-1-(2,3-dichlorophenyl)-4-hydroxy-6-oxo-1,6-dihydro-5-pyrimidinyl]carbonyl}glycine

88a) 2-Cyclohexyl-3-(2,3-dichlorophenyl)-6-hydroxy-4(3H)-pyrimidinone. 1M Dimethylaluminium chloride in hexane (2.75 mL, 2.75 mmol) was added to a stirred solution of 2,3-dichloroaniline (0.405 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.445 g, 53%) as a white solid. 1H NMR (400 MHz, DMSO-d₆) δ ppm 0.81-1.03 (m, 2 H) 1.05-1.15 (m, 1 H) 1.34-1.47 (m, 1 H) 1.49-1.73 (m, 5 H) 1.76-1.85 (m, 1 H) 1.90-2.01 (m, 1 H) 5.37 (s, 1 H) 7.56 (t, J=7.96 Hz, 1 H) 7.63 (dd, J=7.95, 1.65 Hz, 1 H) 7.84 (dd, J=7.96, 1.64 Hz, 1 H) 11.63 (br. s., 1 H).

88b) N-{[2-Cyclohexyl-1-(2,3-dichlorophenyl)-4-hydroxy-6-oxo-1,6-dihydro-5-pyrimidinyl]carbonyl}glycine. A mixture of 2-cyclohexyl-3-(2,3-dichlorophenyl)-6-hydroxy-4(3H)-pyrimidinone (0.444 g, 1.31 mmol), ethyl isocyanatoacetate (0.457 mL, 2.62 mmol), diisopropylethylamine (0.294 mL, 2.62 mmol) and dichloromethane (3 mL) was stirred in a microwave reactor at 140° C. for 1 h, then cooled. Trifluoroacetic acid (0.404 mL, 5.24 mmol) was added and the mixture chromatographed directly (silica gel, 1-9% methanol/dichloromethane) to give the intermediate ester, sufficiently pure for the next step. 1M aqueous sodium hydroxide (10.0 mL, 10.0 mmol) was added slowly to a stirred solution of the intermediate ester in ethanol (40 mL) and the solution stirred for 18 h at room temperature, then diluted with water (150 mL) and acidified to pH 1 with 6M aqueous hydrochloric acid. The mixture was stirred 1 h, then the precipitated solid filtered, washed with water and dried to give the title compound (0.364 g, 63%) as a white solid. 1H NMR (400 MHz, DMSO-d₆) δ ppm 0.83-1.05 (m, 2 H) 1.09-1.22 (m, 1 H) 1.37-1.49 (m, 1 H) 1.51-1.73 (m, 5 H) 1.78-1.89 (m, 1 H) 1.98-2.08 (m, 1 H) 4.03 (dd, J=17.94, 5.56 Hz, 1 H) 4.08 (dd, J=17.94, 5.56 Hz, 1 H) 7.63 (t, J=7.96 Hz, 1 H) 7.79 (dd, J=8.08, 1.52 Hz, 1 H) 7.91 (dd, J=8.21, 1.39 Hz, 1 H) 9.57 (t, J=5.56 Hz, 1 H) 12.93 (br. s., 1 H) 16.16 (s, 1 H).

### EXAMPLE 89

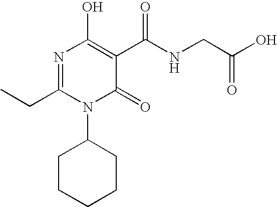

### N-[(1-Cyclohexyl-2-ethyl-4-hydroxy-6-oxo-1,6-dihydro-5-pyrimidinyl)carbonyl]glycine

89a) 3-Cyclohexyl-2-ethyl-6-hydroxy-4(3H)-pyrimidinone. Dimethylaluminium chloride (2.77 ml, 2.77 mmol) was added to a solution of propionitrile (0.216 ml, 3.02 mmol) and cyclohexylamine (0.288 ml, 2.52 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.531 ml, 10.08 mmol) and sodium methoxide (2.307 ml, 10.08 mmol) were added and the mixture was stirred at reflux for 18 h. After cooling, the mixture was poured into water. The pH was adjusted to ca. 3 by te addition of 1 N HCl and extracted with EtOAc. The organics were washed with brine, dried over Na₂SO₄ and evaporated. The oily residue solidify on standing. Triturated from Et₂O and dried to give 3-cyclohexyl-2-ethyl-6-hydroxy-4(3H)-pyrimidinone (320 mg, 1.368 mmol, 54.3% yield) as a white powder. ¹H NMR (400 MHz, CHLOROFORM-d) δ ppm 5.34 (s, 1 H), 3.78-3.96 (m, 1 H), 2.69-3.02 (m, 4 H), 1.88-2.01 (m, 2 H), 1.61-1.78 (m, 3 H), 1.18-1.48 (m, 6 H). LCMS (ES⁺) m/z 223 (MH⁺).

89b) N-[(1-Cyclohexyl-2-ethyl-4-hydroxy-6-oxo-1,6-dihydro-5-pyrimidinyl)carbonyl]glycine. A solution of 3-cyclohexyl-2-ethyl-6-hydroxy-4(3H)-pyrimidinone (317 mg, 1.426 mmol), Hunig's base (0.373 ml, 2.139 mmol) and ethyl isocyanatoacetate (0.240 ml, 2.139 mmol) in dichloromethane (DCM) (3.5 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in Ethanol (7.5 mL) and 1 M NaOH (7.5 ml, 7.50 mmol) and stirred at rt for 2 h. It was then poured into water and acidified by the addition of 1 N HCl. The precipitate was collected washed with water and dried to give N-[(1-cyclohexyl-2-ethyl-4-hydroxy-6-oxo-1,6-dihydro-5-pyrimidinyl)carbonyl]glycine (272 mg, 0.799 mmol, 56.0% yield) as a white powder. ¹H-NMR (400 MHz, DMSO-d₆) δ ppm 15.68 (s, 1 H), 12.86 (s, 1 H), 9.87 (t, J=5.56 Hz, 1 H), 4.07-4.19 (m, 1 H), 4.05 (d, J=5.56 Hz, 2 H), 2.90 (q, J=7.33 Hz, 2 H), 2.50-2.62 (m, 2 H), 1.69-1.86 (m, 4 H), 1.58-1.69 (m, 1 H), 1.28-1.46 (m, 2 H), 1.19 (t, J=7.20 Hz, 3 H), 1.08-1.15 (m, 1 H). LCMS (ES⁻) m/z 324 (MH⁺).

### EXAMPLE 90

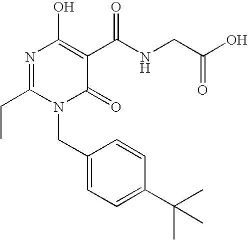

### N-[(1-{[4-(1,1-Dimethylethyl)phenyl]methyl}-2-ethyl-4-hydroxy-6-oxo-1,6-dihydro-5-pyrimidinyl)carbonyl]glycine

Dimethylaluminium chloride (2.423 ml, 2.423 mmol) was added to a solution of propionitrile (0.189 ml, 2.64 mmol) and 4-t-butylbenzylamine (0.388 ml, 2.203 mmol) in toluene (2.8 ml). The resulting mixture was stirred under nitrogen for 10 min. and then at 150° C. for 30 min i n a Biotage Initiator® microwave synthesizer. The reaction mixture was cooled and the solvent evaporated. The residue was suspended in methoxyethanol (8 ml). Diethylmalonate (1.338 ml, 8.81 mmol) and sodium methoxide (2.017 ml, 8.81 mmol) were added and the mixture was stirred at reflux for 18 h. After cooling, the mixture was poured into water. The pH was adjusted to ca. 3 by te addition of 1 N HCl and extracted with EtOAc. The organics were washed with brine, dried over Na₂SO₄ and evaporated. Oily residue, solidify on standing (282 mg). A solution of the above product, Hunig's base (0.384 ml, 2.203 mmol) and ethyl isocyanatoacetate (0.247 ml, 2.203 mmol) in dichloromethane (DCM) (4 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 ml) and 1 M NaOH (10 ml, 10.00 mmol) and stirred at rt for 2 h. It was then poured into water and acidified by the addition of 1 N HCl. The precipitate was collected by filtration and purified by RP-HPLC (20-95% acetonitrile in water plus 0.1% TFA) to afford N-[(1-{[4-(1,1-dimethylethyl)phenyl]methyl}-2-ethyl-4-hydroxy-6-oxo-1,6-dihydro-5-pyrimidinyl)carbonyl]glycine (141 mg, 0.346 mmol, 15.69% yield). ¹H-NMR (400 MHz, DMSO-d₆) δ ppm 15.85 (br. s., 1 H), 12.89 (br. s., 1 H), 9.83 (t, J=5.56 Hz, 1 H), 7.37 (d, J=8.34 Hz, 2 H), 7.11 (d, J=8.59 Hz, 2 H), 5.26 (s, 2 H), 4.08 (d, J=5.56 Hz, 2 H), 2.77 (q, J=7.24 Hz, 2 H), 1.25 (s, 9 H), 1.12 (t, J=7.20 Hz, 3 H). LCMS (ES^{|}) m/z 388 (MH^{|}).

### EXAMPLE 91

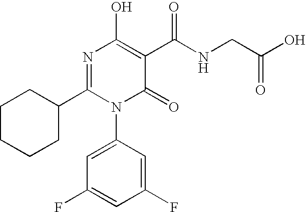

### N-{[2-Cyclohexyl-1-(3,5-difluorophenyl)-4-hydroxy-6-oxo-1,6-dihydro-5-pyrimidinyl]carbonyl}glycine

91a) 2-Cyclohexyl-3-(3,5-difluorophenyl)-6-hydroxy-4(3H)-pyrimidinone. 1M Dimethylaluminium chloride in hexane (2.75 mL, 2.75 mmol) was added to a stirred solution of 3,5-difluoroaniline (0.323 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.182 g, 24%) as a white solid. 1H NMR (400 MHz, DMSO-d₆) δ ppm 0.87-1.01 (m, 2 H) 1.04-1.19 (m, 1 H) 1.40-1.60 (m, 3 H) 1.61-1.70 (m, 2 H) 1.74-1.84 (m, 2 H) 2.10 (tt, J=11.49, 3.16 Hz, 1 H) 5.32 (s, 1 H) 7.30-7.39 (m, 2 H) 7.46 (tt, J=9.38, 2.37 Hz, 1 H) 11.45 (br. s., 1 H).

91b) N-{[2-Cyclohexyl-1-(3,5-difluorophenyl)-4-hydroxy-6-oxo-1,6-dihydro-5-pyrimidinyl]carbonyl}glycine. A mixture of 2-cyclohexyl-3-(3,5-difluorophenyl)-6-hydroxy-4(3H)-pyrimidinone (0.180 g, 0.588 mmol), ethyl isocyanatoacetate (0.132 mL, 1.18 mmol), diisopropylethylamine (0.205 mL, 1. 18 mmol) and dichloromethane (1.5 mL) was stirred in a microwave reactor at 140° C. for 1 h, then cooled. Trifluoroacetic acid (0.182 mL, 2.36 mmol) was added and the mixture chromatographed directly (silica gel, 1-9% methanol/dichloromethane) to give the intermediate ester, sufficiently pure for the next step. 1M aqueous sodium hydroxide (3.80 mL, 3.80 mmol) was added slowly to a stirred solution of the intermediate ester in ethanol (15 mL) and the solution stirred for 18 h at room temperature, then diluted with water (60 mL) and acidified to pH 1 with 6M aqueous hydrochloric acid. The mixture was stirred 1 h, then the precipitated solid filtered, washed with water and dried to give the title compound (0.072 g, 30%) as a white solid. 1H NMR (400 MHz, DMSO-d₆) δ ppm 0.87-1.03 (m, 2 H) 1.09-1.22 (m, 1 H) 1.42-1.60 (m, 3 H) 1.63-1.73 (m,2 H) 1.74-1.87 (m, 2 H) 2.14 (tt, J=11.37, 3.03 Hz, 1 H) 4.05 (d, J=5.56 Hz, 2 H) 7.45-7.60 (m, 3 H) 9.63 (t, J=5.68 Hz, 1 H) 12.91 (br. s., 1 H) 16.00 (s, 1 H).

### EXAMPLE 92

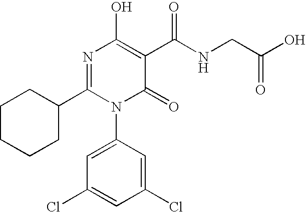

### N-{[2-Cyclohexyl-1-(3,5-dichlorophenyl)-4-hydroxy-6-oxo-1,6-dihydro-5-pyrimidinyl]carbonyl}glycine

92a) 2-Cyclohexyl-3-(3,5-dichlorophenyl)-6-hydroxy-4(3H)-pyrimidinone. 1M Dimethylaluminium chloride in hexane (2.75 mL, 2.75 mmol) was added to a stirred solution of 3,5-dichloroaniline (0.405 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.155 g, 18%) as a cream solid. 1H NMR (400 MHz, DMSO-d₆) δ ppm 0.86-1.03 (m, 2 H) 1.05-1.18 (m, 1 H) 1.41-1.60 (m, 3 H) 1.61-1.71 (m, 2 H) 1.74-1.84 (m, 2 H) 2.02-2.12 (m, 1 H) 5.32 (s, 1 H) 7.66 (d, J=1.77 Hz, 2 H) 7.80 (t, J=1.89 Hz, 1 H) 11.48 (br. s., 1 H).

92b) N-{[2-Cyclohexyl-1-(3,5-dichlorophenyl)-4-hydroxy-6-oxo-1,6-dihydro-5-pyrimidinyl]carbonyl}glycine. A mixture of 2-cyclohexyl-3-(3,5-dichlorophenyl)-6-hydroxy-4(3H)-pyrimidinone (0.153 g, 0.451 mmol), ethyl isocyanatoacetate (0.101 mL, 0.902 mmol), diisopropylethylamine (0.158 mL, 0.902 mmol) and dichloromethane (1.5 mL) was stirred in a microwave reactor at 140° C. for 1 h, then cooled. Trifluoroacetic acid (0.139 mL, 1.80 mmol) was added and the mixture chromatographed directly (silica gel, 1-9% methanol/dichloromethane) to give the intermediate ester, sufficiently pure for the next step. 1M aqueous sodium hydroxide (3.60 mL, 3.60 mmol) was added slowly to a stirred solution of the intermediate ester in ethanol (15 mL) and the solution stirred for 18 h at room temperature, then diluted with water (80 mL) and acidified to pH 1 with 6M aqueous hydrochloric acid. The mixture was stirred 1 h, then the precipitate filtered, washed with water and dried. The solid was purified by reverse-phase preparative HPLC (ODS, 10-90% acetonitrile/water+0.1% trifluoroacetic acid) to give the title compound (0.075 g, 38%) as a cream solid. 1H NMR (400 MHz, DMSO-d₆) δ ppm 0.89-1.03 (m, 2 H) 1.10-1.23 (m, 1 H) 1.42-1.60 (m, 3 H) 1.62-1.72 (m, 2 H) 1.76-1.85 (m, 2 H) 2.12 (tt, J=11.50, 3.16 Hz, 1 H) 4.05 (d, J=5.81 Hz, 2 H) 7.80 (d, J=1.77 Hz, 2 H) 7.88 (t, J=1.77 Hz, 1 H) 9.62 (t, J=5.68 Hz, 1 H) 12.89 (br. s., 1 H) 15.99 (s, 1 H).

### EXAMPLE 93

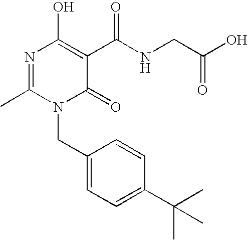

### N-[(1-{[4-(1,1-Dimethylethyl)phenyl]methyl}-4-hydroxy-2-methyl-6-oxo-1,6-dihydro-5-pyrimidinyl)carbonyl]glycine

93a) 3-{[4-(1,1-Dimethylethyl)phenyl]methyl -6-hydroxy-2-methyl-4(3H)-pyrimidinone. Dimethylaluminium chloride (2.423 ml, 2.423 mmol) was added to a solution of acetonitrile (0.138 ml, 2.64 mmol) and 4-t-butylbenzylamine (0.388 ml, 2.203 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.338 ml, 8.81 mmol) and sodium methoxide (2.017 ml, 8.81 mmol) were added and the mixture was stirred at reflux for 18 h. After cooling, the mixture was poured into water. The pH was adjusted to ca. 3 by te addition of 1 N HCl and extracted with EtOAc. The organics were washed with brine, dried over Na₂SO₄ and evaporated. The residue was purified on silica gel (0-9% MeOH in CHCl₃). No improvement in product purity was achieved (by LC/MS). ¹H-NMR in CDCl₃ is consistent with desired product [3-{[4-(1,1-dimethylethyl)phenyl]methyl}-6-hydroxy-2-methyl-4(3H)-pyrimidinone (798 mg, 1.905 mmol, 86% yield)]. Purity by LC/MS was 65%. The title compound was used as is in the next step. ¹H NMR (400 MHz, CHLOROFORM-d) δ ppm 7.36 (ABq, JAB=8.34 Hz, 2 H), 7.14 (ABq, JAB=8.59 Hz, 2 H), 5.54 (s, 1 H), 5.25 (s, 2 H), 2.48 (s, 3 H), 1.30 (s, 9 H). LCMS (ES⁺) m/z 273 (MH⁺).

93b) N-[(1-{[4-(1,1-Dimethylethyl)phenyl]methyl}-4-hydroxy-2-methyl-6-oxo-1,6-dihydro-5-pyrimidinyl)carbonyl]glycine. A solution of 3-{[4-(1,1-dimethylethyl)phenyl]methyl}-6-hydroxy-2-methyl-4(3H)-pyrimidinone (798 mg, 2.93 mmol), Hunig's base (0.612 ml, 3.52 mmol) and ethyl isocyanatoacetate (0.394 ml, 3.52 mmol) in dichloromethane (4 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 ml) and 1 M NaOH (10 ml, 10.00 mmol) and stirred at rt for 2 h. It was then poured into water and acidified by the addition of 1 N HCl. The precipitate was collected by filtration (520 mg, yellow powder, 85% pure by LC/MS). Trituration from hot toluene afforded N-[(2-(3,5-dichloro-4-pyridinyl)-1-{[4-(1,1-dimethylethyl)phenyl]methyl}-4-hydroxy-6-oxo-1,6-dihydro-5-pyrimidinyl)carbonyl]glycine (255 mg, 0.649 mmol, 22.14% yield). ¹H-NMR (400 MHz, DMSO-d₆) δ ppm 15.83 (s, 1 H), 12.89 (br. s., 1 H), 9.81 (t, J=5.43 Hz, 1 H), 7.32-7.44 (m, 2 H), 7.14 (d, J=8.34 Hz, 2 H) 5.24 (s, 2 H), 4.07 (d, J=5.56 Hz, 2 H), 2.48 (s, 3 H), 1.25 (s, 9 H). LCMS (ES^{|}) m/z 374 (MH^{|}).

### EXAMPLE 94

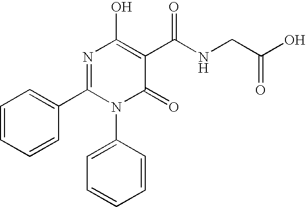

### N-[(4-Hydroxy-6-oxo-1,2-diphenyl-1,6-dihydro-5-pyrimidinyl)carbonyl]glycine

94a) 6-Hydroxy-2,3-diphenyl-4(3H)-pyrimidinone. 1M Dimethylaluminium chloride in hexane (2.75 mL, 2.75 mmol) was added to a stirred solution of aniline (0.233 g, 2.50 mmol) and benzonitrile (0.309 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 1 0.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.206 g, 31%) as a cream solid. 1H NMR (400 MHz, DMSO-d₆) δ ppm 5.48 (s, 1 H) 7.15-7.36 (m, 10 H) 11.73 (br. s., 1 H).

94b) N-[(4-Hydroxy-6-oxo-1,2-diphenyl-1,6-dihydro-5-pyrimidinyl)carbonyl]glycine. A mixture of 6-hydroxy-2,3-diphenyl-4(3H)-pyrimidinone (0.204 g, 0.772 mmol), ethyl isocyanatoacetate (0. 173 mL, 1.54 mmol), diisopropylethylamine (0.270 mL, 1.54 mmol) and dichloromethane (3.0 mL) was stirred in a microwave reactor at 140° C. for 1 h, then cooled. Trifluoroacetic acid (0.237 mL, 3.08 mmol) was added and the mixture chromatographed directly (silica gel, 1-9% methanol/dichloromethane) to give the intermediate ester, sufficiently pure for the next step. 1M aqueous sodium hydroxide (5.50 mL, 5.50 mmol) was added slowly to a stirred solution of the intermediate ester in ethanol (20 mL) and the solution stirred for 1 h at room temperature, then diluted with water (15 mL) to dissolve the precipitated solid. Stirring was continued for 1 h, then the mixture diluted further with water (80 mL) and acidified to pH 1 with 6M aqueous hydrochloric acid. The mixture was stirred 0.5 h, then the precipitated solid filtered, washed with water and dried to give the title compound (0.167 g, 59%) as a white solid. 1H NMR (400 MHz, DMSO-d₆) δ ppm 4.10 (d, J=5.56 Hz, 2 H) 7.19-7.41 (m, 10 H) 9.81 (t, J=5.56 Hz, 1 H) 12.94 (br. s., 1 H) 16.12 (br. s., 1 H).

### EXAMPLE 95

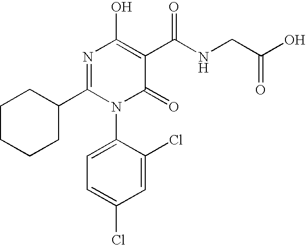

### N-{[2-Cyclohexyl-1-(2,4-dichlorophenyl)-4-hydroxy-6-oxo-1,6-dihydro-5-pyrimidinyl]carbonyl}glycine

95a) 2-Cyclohexyl-3-(2,4-dichlorophenyl)-6-hydroxy-4(3H)-pyrimidinone. 1M Dimethylaluminium chloride in hexane (2.75 mL, 2.75 mmol) was added to a stirred solution of 2,4-dichloroaniline (0.405 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.312g, 37%) as a white solid. 1H NMR(400 MHz, DMSO-d₆) δ ppm 0.83-1.19 (m, 3 H) 1.33-1.47 (m, 1 H) 1.51-1.72 (m, 5 H) 1.74-1.84 (m, 1 H) 1.93-2.03 (m, 1 H) 5.36 (s, 1 H) 7.61-7.69 (m, 2 H) 7.94 (d, J=1.77 Hz, 1 H) 11.59 (br. s., 1 H).

95b) N-{[2-Cyclohexyl-1-(2,4-dichlorophenyl)-4-hydroxy-6-oxo-1,6-dihydro-5-pyrimidinyl]carbonyl}glycine. A mixture of 2-cyclohexyl-3-(2,4-dichlorophenyl)-6-hydroxy-4(3H)-pyrimidinone (0.310 g, 0.914 mmol), ethyl isocyanatoacetate (0.205 mL, 1.83 mmol), diisopropylethylamine (0.319 mL, 1.83 mmol) and dichloromethane (3.0 mL) was stirred in a microwave reactor at 140° C. for 1 h, then cooled. Trifluoroacetic acid (0.282 mL, 3.66 mmol) was added and the mixture chromatographed directly (silica gel, 1-9% methanol/dichloromethane) to give the intermediate ester, sufficiently pure for the next step. 1M aqueous sodium hydroxide (7.00 mL, 7.00 mmol) was added slowly to a stirred solution of the intermediate ester in ethanol (30 mL) and the solution stirred for 2 h at room temperature, then diluted with water (100 mL) and acidified to pH 1 with 6M aqueous hydrochloric acid. The mixture was stirred 0.5 h, then the precipitated solid filtered, washed with water and dried to give the title compound (0.278 g, 69%) as a white solid. 1H NMR (400 MHz, DMSO-d₆) δ ppm 0.84-1.06 (m, 2 H) 1.09-1.22 (m, 1 H) 1.35-1.49 (m, 1 H) 1.50-1.74 (m, 5 H) 1.77-1.87 (m, 1 H) 2.05 (tt, J=11.24, 3.16 Hz, 1 H) 4.02 (dd, J=18.19, 5.56 Hz, 1 H) 4.07 (dd, J=18.19, 5.81 Hz, 1 H) 7.72 (dd, J=8.34, 2.27 Hz, 1 H) 7.82 (d, J=8.59 Hz, 1 H) 8.02 (d, J=2.27 Hz, 1 H) 9.58 (t, J=5.43 Hz, 1 H) 12.94 (br. s., 1 H) 16.16 (br. s., 1 H).

### EXAMPLE 96

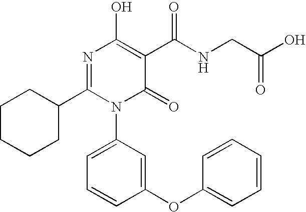

### N-({2-Cyclohexyl-4-hydroxy-6-oxo-1-[3-(phenyloxy)phenyl]-1,6-dihydro-5-pyrimidinyl}carbonyl)glycine

96a) 2-Cyclohexyl-6-hydroxy-3-[3-(phenyloxy)phenyl]-4(3H)-pyrimidinone. 1M Dimethylaluminium chloride in hexane (2.75 mL, 2.75 mmol) was added to a stirred solution of 3-phenoxyaniline (0.463 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.189 g, 21%) as an off-white solid. 1H NMR (400 MHz, DMSO-d₆) δ ppm 0.83-1.02 (m, 2 H) 1.05-1.19 (m, 1 H) 1.34-1.61 (m, 3 H) 1.62-1.80 (m, 4 H) 2.11-2.20 (m, 1 H) 5.29 (s, 1 H) 6.99-7.06 (m, 3 H) 7.08-7.14 (m, 1 H) 7.14-7.21 (m, 2 H) 7.37-7.45 (m, 2 H) 7.55 (t, J=8.08 Hz, 1 H) 11.35 (br. s., 1 H).

96b) N-({2-Cyclohexyl-4-hydroxy-6-oxo-1-[3-(phenyloxy)phenyl]-1,6-dihydro-5-pyrimidinyl}carbonyl)glycine. A mixture of 2-cyclohexyl-6-hydroxy-3-[3-(phenyloxy)phenyl]-4(3H)-pyrimidinone (0.187 g, 0.516 mmol), ethyl isocyanatoacetate (0.116 mL, 1.03 mmol), diisopropylethylamine (0.180 mL, 1.03 mmol) and dichloromethane (2.0 mL) was stirred in a microwave reactor at 140° C. for 1 h, then cooled. Trifluoroacetic acid (0.159 mL, 2.06 mmol) was added and the mixture chromatographed directly (silica gel, 1-9% methanol/dichloromethane) to give the intermediate ester, sufficiently pure for the next step. 1M aqueous sodium hydroxide (4.00 mL, 4.00 mmol) was added slowly to a stirred suspension of the intermediate ester in ethanol (16 mL) and the solution stirred for 2 h at room temperature, then diluted with water (80 mL) and acidified to pH 1 with 6M aqueous hydrochloric acid. The mixture was stirred 0.5 h, then the precipitated solid filtered, washed with water and dried to give the title compound (0.149 g, 62%) as a white solid. 1H NMR (400 MHz, DMSO-d₆) δ ppm 0.86-1.02 (m, 2 H) 1.08-1.22 (m, 1 H) 1.36-1.61 (m, 3 H) 1.62-1.83 (m, 4 H) 2.18 (tt, J=11.49, 2.91 Hz, 1 H) 4.04 (d, J=5.56 Hz, 2 H) 7.00-7.06 (m, 2 H) 7.13-7.30 (m, 4 H) 7.38-7.47 (m, 2 H) 7.59 (t, J=7.96 Hz, 1 H) 9.69 (t, J=5.68 Hz, 1 H) 12.89 (br. s., 1 H) 15.90 (s, 1 H).

### EXAMPLE 97

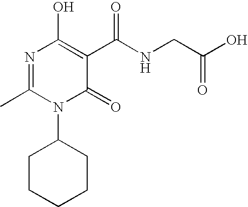

### N-[(1-Cyclohexyl-4-hydroxy-2-methyl-6-oxo-1,6-dihydro-5-pyrimidinyl)carbonyl]glycine

Dimethylaluminium chloride (2.77 ml, 2.77 mmol) was added to a solution of acetonitrile (0.158 ml, 3.02 mmol) and cyclohexylamine (0.288 ml, 2.52 mmol) in toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.531 ml, 10.08 mmol) and sodium methoxide (2.307 ml, 10.08 mmol) were added and the mixture was stirred at reflux for 18 h. After cooling, the mixture was poured into water. The pH was adjusted to ca. 3 by the addition of 1 N HCl and extracted with EtOAc. The organics were washed with brine, dried over Na₂SO₄ and evaporated. The oily residue solidify on standing. Triturated from Et₂O and dried to give 3-cyclohexyl-6-hydroxy-2-methyl-4(3H)-pyrimidinone as a white powder. A solution of the above 3-cyclohexyl-6-hydroxy-2-methyl-4(3H)-pyrimidinone in dichloromethane (DCM) (4 ml) and Hunig's base (0.659 ml, 3.78 mmol) was treated with ethyl isocyanatoacetate (0.424 ml, 3.78 mmol) and irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 ml) and 1 M NaOH (10 ml, 10.00 mmol) and stirred at rt for 2 h. It was then poured into water and acidified by the addition of 1 N HCl. The precipitate was collected by filtration, triturated from EtOAc and dried to give N-[(1-cyclohexyl-4-hydroxy-2-methyl-6-oxo-1,6-dihydro-5-pyrimidinyl)carbonyl]glycine (102 mg, 0.313 mmol, 12.43% yield) as a white powder. ¹H-NMR (400 MHz, DMSO-d₆) δ ppm 15.67 (s, 1 H), 12.86 (br. s., 1 H), 9.85 (t, J=5.43 Hz, 1 H), 4.00-4.07 (m, 1 H), 4.05 (d, J=5.56 Hz, 2 H), 2.59 (s, 3 H), 2.42-2.54 (m, 2 H), 1.69-1.85 (m, 4 H), 1.57-1.69 (m, 1 H), 1.27-1.45 (m, 2 H), 1.05-1.23 (m, 1 H). LCMS (ES⁺) m/z 310 (MH⁺).

### EXAMPLE 98

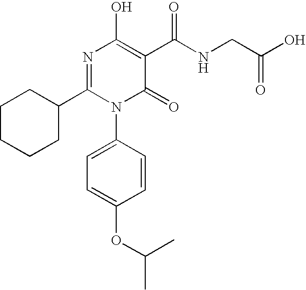

### N-[(2-Cyclohexyl-4-hydroxy-1-{4-[(1-methylethyl)oxy]phenyl}-6-oxo-1,6-dihydro-5-pyrimidinyl)carbonyl]glycine

98a) 2-Cyclohexyl-6-hydroxy-3-{4-[(1-methylethyl)oxy]phenyl}-4(3H)-pyrimidinone. 1M Dimethylaluminium chloride in hexane (2.75 mL, 2.75 mmol) was added to a stirred solution of 4-isopropoxyaniline (0.378 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.52 mL, 10.0 mmol) added, followed by 2-methoxyethanol (5 mL) and methanolic sodium methoxide (2.30 mL, 10.0 mmol) and the mixture refluxed under nitrogen for 18 h, then cooled and poured into water (70 mL). The mixture was washed with ether, then acidified to pH 1 with 6M aqueous hydrochloric acid and extracted again with ethyl acetate. The extracts contained none of the required pyrimidinedione (TLC). The ether wash was dried (Na₂SO₄) and evaporated under reduced pressure to leave a waxy solid (643 mg), fairly pure intermediate amidine by LCMS. Potassium tert-butoxide (0.554 g, 4.94 mmol) was added to a solution of this amidine and diethyl malonate (0.753 mL, 4.94 mmol) in 2-methoxyethanol (5 mL) and the mixture heated in a microwave synthesiser at 200° C. for 0.5 h, then cooled and poured into 0.1 M aqueous sodium hydroxide (50 mL). The mixture was washed with ether, then acidified with 6 M aqueous hydrochloric acid to pH 1 and extracted with ethyl acetate. The extracts were washed with water, brine, dried (MgSO₄) and evaporated under reduced pressure. The residue was chromatographed (silica gel, 1-9% methanol/dichloromethane) and the product triturated with ether, then dried to give the title compound (0.101 g, 12%) as a tan solid. 1 H NMR (400 MHz, DMSO-d₆) ≢ ppm 0.80-0.96 (m, 2 H) 1.04-1.16 (m, 1 H) 1.31 (d, J=6.06 Hz, 6 H) 1.42-1.58 (m, 3 H) 1.59-1.78 (m, 4 H) 2.10-2.21 (m, 1 H) 4.63-4.75 (sept, J=6.06 Hz, 1 H) 5.29 (s, 1 H) 6.99-7.06 (m, 2 H) 7.15-7.22 (m, 2 H) 11.28 (br. s., 1H).

98b) N-[(2-Cyclohexyl-4-hydroxy-1-{4-[(1-methylethyl)oxy]phenyl}-6-oxo-1,6-dihydro-5-pyrimidinyl)carbonyl]glycine. A mixture of 2-cyclohexyl-6-hydroxy-3-{4-[(1-methylethyl)oxy]phenyl}-4(3H)-pyrimidinone (0.101 g, 0. 308 mmol), ethyl isocyanatoacetate (0.069 mL, 0.615 mmol), diisopropylethylamine (0.107 mL, 0.615 mmol) and dichloromethane (1 mL) was stirred in a microwave reactor at 140° C. for 1 h, then cooled. Trifluoroacetic acid (0.095 mL, 1.23 mmol) was added and the mixture chromatographed directly (silica gel, 1-9% methanol/dichloromethane) to give the intermediate ester, sufficiently pure for the next step (LCMS). 1M aqueous sodium hydroxide (1.50 mL, 1.50 mmol) was added slowly to a stirred solution of the intermediate ester in ethanol (6 mL) and the solution stirred for 18 h at room temperature, then diluted with water (80 mL) and acidified to pH 1 with 6M aqueous hydrochloric acid. The mixture was stirred 0.5 h, then the precipitated solid filtered, washed with water and dried to give the title compound (0.054 g, 41%) as a white solid. 1H NMR (400 MHz, DMSO-d₆) δ ppm 0.81-0.96 (m, 2 H) 1.04-1.21 (m, 1 H) 1.31 (d, J=6.06 Hz, 6 H) 1.42-1.58 (m, 3 H) 1.61-1.69 (m, 2 H) 1.71-1.82 (m, 2 H) 2.12-2.25 (m, 1 H) 4.04 (d, J=5.56 Hz, 2 H) 4.62-4.77 (sept, J=6.06 Hz, 1 H) 7.03-7.11 (m, 2 H) 7.31-7.39 (m, 2 H) 9.74 (t, J=5.56 Hz, 1 H) 12.89 (br. s., 1 H) 15.87 (s, 1 H).

### EXAMPLE 99

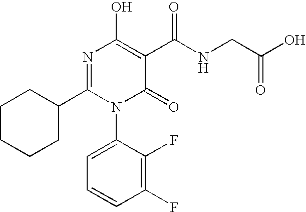

### N-{[2-Cyclohexyl-1-(2,3-difluorophenyl)-4-hydroxy-6-oxo-1,6-dihydro-5-pyrimidinyl]carbonyl}glycine

99a) 2-Cyclohexyl-3-(2,3-difluorophenyl)-6-hydroxy-4(3H)-pyrimidinone. 1M Dimethylaluminium chloride in hexane (2.75 mL, 2.75 mmol) was added to a stirred solution of 2,3-difluoroaniline (0.323 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.53 mL, 10.0 mmol) added, followed by 2-methoxyethanol (5 mL) and 4.37 M methanolic sodium methoxide (2.29 mL, 10.0 mmol) and the mixture stirred at reflux under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.168 g, 22%) as a white solid. 1H NMR (400 MHz, DMSO-d₆) δ ppm 0.84-1.03 (m, 2 H) 1.06-1.19 (m, 1 H) 1.40-1.73 (m, 6 H) 1.78-1.88 (m, 1 H) 2.09-2.20 (m, 1 H) 5.37 (s, 1 H) 7.35-7.48 (m, 2 H) 7.57-7.74 (m, 1 H) 11.66 (br. s., 1 H).

99b) N-{[2-Cyclohexyl-1-(2,3-difluorophenyl)-4-hydroxy-6-oxo-1,6-dihydro-5-pyrimidinyl]carbonyl}glycine. A mixture of 2-cyclohexyl-3-(2,3-difluorophenyl)-6-hydroxy-4(3H)-pyrimidinone (0.166 g, 0.542 mmol), ethyl isocyanatoacetate (0.122 mL, 1.08 mmol), diisopropylethylamine (0.189 mL, 1.08 mmol) and dichloromethane (1.5 mL) was stirred in a microwave reactor at 140° C. for 1 h, then cooled. Trifluoroacetic acid (0.166 mL, 2.16 mmol) was added and the mixture chromatographed directly (silica gel, 1-9% methanol/dichloromethane) to give the intermediate ester, sufficiently pure for the next step (LCMS). 1M aqueous sodium hydroxide (4.00 mL, 4.00 mmol) was added slowly to a stirred solution of the intermediate ester in ethanol (16 mL) and the solution stirred for 3 h at room temperature, then diluted with water (100 mL) and acidified to pH 1 with 6M aqueous hydrochloric acid. The mixture was stirred 0.5 h, then the precipitated solid filtered, washed with water and dried to give the title compound (0.129 g, 58%) as a white solid. 1H NMR (400 MHz, DMSO-d₆) δ ppm 0.85-1.06 (m, 2 H) 1.08-1.21 (m, 1 H) 1.41-1.73 (m, 6 H) 1.81-1.92 (m, 1 H) 2.16-2.28 (m, 1 H) 4.05 (d, J=5.81 Hz, 2 H) 7.43-7.51 (m, 1 H) 7.54-7.61 (m, 1 H) 7.67-7.80 (m, 1 H) 9.57 (t, J=5.56 Hz, 1 H) 12.93 (br. s., 1 H) 16.19 (s, 1 H).

### EXAMPLE 100

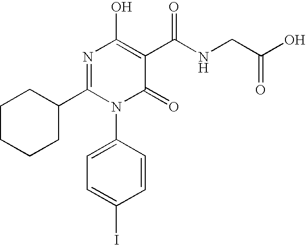

### N-{[2-Cyclohexyl-4-hydroxy-1-(4-iodolphenyl)-6-oxo-1,6-dihydro-5-pyrimidinyl]carbonyl}glycine

100a) 2-Cyclohexyl-6-hydroxy-3-(4-iodophenyl)-4(3H)-pyrimidinone. 1M Dimethylaluminium chloride in hexane (2.75 mL, 2.75 mmol) was added to a stirred solution of 4-iodoaniline (0.548 g, 2.50 mmol) and cyclohexanecarbonitrile (0.328 g, 3.00 mmol) in toluene (2 mL) and the mixture stirred for 15 min at room temperature under nitrogen, then microwaved at 150° C. for 30 min. After cooling, the solvent was removed under reduced pressure and diethyl malonate (1.53 mL, 10.0 mmol) added, followed by 2-methoxyethanol (5 mL) and 4.37 M methanolic sodium methoxide (2.29 mL, 10.0 mmol) and the mixture stirred at reflux under nitrogen for 18 h, then cooled and poured into water (100 mL). The mixture was washed with ether, then acidified to pH 1 with 6M aqueous hydrochloric acid and extracted again with ethyl acetate. The extracts were washed with water, brine, dried (MgSO₄) and evaporated under reduced pressure. The residue was chromatographed (silica gel, 1-9% methanol/dichloromethane) and the product triturated with ether. The solid was collected, washed with ether and dried to give the title compound (0.225 g, 23%) as a white solid. 1H NMR (400 MHz, DMSO-d₆) δ ppm 0.81-0.99 (m, 2 H) 1.04-1.19 (m, 1 H) 1.37-1.59 (m, 3 H) 1.61-1.68 (m, 2 H) 1.70-1.79 (m, 2 H) 2.11 (tt, J=11.49, 3.28 Hz, 1 H) 5.30 (s, 1 H) 7.11-7.20 (m, 2 H) 7.85-7.93 (m, 2 H) 11.40 (br. s., 1 H).

100b) N-{[2-Cyclohexyl-4-hydroxy-1-(4-iodophenyl)-6-oxo-1,6-dihydro-5-pyrimidinyl]carbonyl}glycine. A mixture of 2-cyclohexyl-6-hydroxy-3-(4-iodophenyl)-4(3H)-pyrimidinone (0.222 g, 0.560 mmol), ethyl isocyanatoacetate (0.126 mL, 1.12 mmol), diisopropylethylamine (0.196 mL, 1.12 mmol) and dichloromethane (1.5 mL) was stirred in a microwave reactor at 140° C. for 1 h, then cooled. Trifluoroacetic acid (0.173 mL, 2.24 mmol) was added and the mixture chromatographed directly (silica gel, 1-9% methanol/dichloromethane) to give the intermediate ester, sufficiently pure for the next step (LCMS). 1M aqueous sodium hydroxide (4.50 mL, 4.50 mmol) was added slowly to a stirred suspension of the intermediate ester in ethanol (18 mL) and the solution stirred for 2 h at room temperature. The precipitated solid was filtered, washed with 5% aqueous ethanol and dried to give the title compound (0.188 g, 62%) as a white solid. 1H NMR (400 MHz, DMSO-d₆) δ ppm 0.78-0.94 (m, J=13.26, 13.26 Hz, 2 H) 1.00-1.18 (m, 2 H) 1.39-1.55 (m, 3 H) 1.57-1.69 (m, 4 H) 1.87-1.98 (m, 1 H) 3.44 (d, J=4.29 Hz, 2 H) 6.96-7.08 (m, J=8.59 Hz, 2 H) 7.78-7.87 (m, 2 H) 10.17 (t, J=4.29 Hz, 1 H).

### EXAMPLE 101

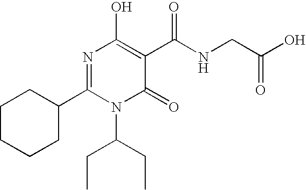

### N-{[2-Cyclohexyl-1-(1-ethylpropyl)-4-hydroxy-6-oxo-1,6-dihydro-5-pyrimidinyl]carbonyl}glycine

101a) 2-Cyclohexyl-3-(1-ethylpropyl)-6-hydroxy-4(3H)-pyrimidinone. Dimethylaluminium chloride (2.75 ml, 2.75 mmol) was added to a solution of cyclohexylcarbonitrile (0.356 ml, 3.00 mmol) and 3-aminopentane (0.291 ml, 2.5 mmol) in Toluene (2.8 ml). The resulting mixture was stirred under nitrogen for 10 min. and then at 150° C. for 30 min in a Biotage Initiator® microwave synthesizer. The reaction mixture was cooled and the solvent evaporated. The residue was suspended in methoxyethanol (8 ml). Diethylmalonate (1.518 ml, 10.00 mmol) and sodium methoxide (2.288 ml, 10.00 mmol) were added and the mixture was stirred at reflux for 18 h. After cooling, the mixture was poured into water. The pH was adjusted to ca. 3 by the addition of 1 N HCl, the precipitate was collected by filtration, washed with water and dried to give 2-cyclohexyl-3-(1-ethylpropyl)-6-hydroxy-4(3H)-pyrimidinone (320 mg, 1.210 mmol, 48.4% yield) as a light pink powder. LCMS (ES^{|}) m/z 265 (MH^{|}).

101b) N-{[2-Cyclohexyl-1-(1-ethylpropyl)-4-hydroxy-6-oxo-1,6-dihydro-5-pyrimidinyl]carbonyl}glycine. A solution of 2-cyclohexyl-3-(1-ethylpropyl)-6-hydroxy-4(3H)-pyrimidinone (315 mg, 1.192 mmol), Hunig's base (0.270 ml, 1.549 mmol) and ethyl isocyanatoacetate (0.174 ml, 1.549 mmol) in dichloromethane (4 ml) was irradiated at 130° C. for 1 h in a Biotage Initiator® microwave synthesizer. The reaction mixture was diluted with dichloromethane and washed with 1 N HCl. The organic phase was dried over Na₂SO₄ and evaporated. The residue was dissolved in ethanol (10 ml) and 1 M NaOH (10 ml, 10.00 mmol) and stirred at rt for 2 h. It was then poured into water and acidified by the addition of 1 N HCl. The solid collected was purified by RP-HPLC (20-95% acetonitrile in water, plus 0.1% TFA) to afford N-{[2-cyclohexyl-1-(1-ethylpropyl)-4-hydroxy-6-oxo-1,6-dihydro-5-pyrimidinyl]carbonyl}glycine (206 mg, 0.547 mmol, 45.9% yield) as a white powder. Mixture of rotomers (2:1 in CDCl₃). ¹H-NMR (400 MHz, CHLOROFORM-d) δ ppm 15.26 (br. s., 0.33 H), 15.11 (br. s., 0.66 H), 10.08 (t, J=4.80 Hz, 0.33 H), 10.02 (t, J=5.05 Hz, 0.66 H), 5.38-5.51 (m, 0.33 H), 4.25 (d, J=6.82 Hz, 0.67 H), 4.24 (d, J=6.82 Hz, 1.33 H), 4.00-4.13 (m, 0.67 H), 2.73-2.95 (m, 1 H), 2.17-2.33 (m, 1 H), 1.67-2.09 (m, 10 H), 1.21-1.43 (m, 3 H), 0.93 (t, J=7.33 Hz, 2 H), 0.89 (t, J=7.33 Hz, 4 H), LCMS (ES) m/z 366 (MH^{|}).

### Biological Background:

The following references set out information about the target enzymes, HIF prolyl hydroxylases, and methods and materials for measuring inhibition of same by small molecules.

M. Hirsilä, P. Koivunen, V. Güzler, K. I. Kivirikko, and J. Myllyhaiju “Characterization of the Human Prolyl 4-Hydroxylases That Modify the Hypoxia-inducible Factor” J. Biol Chem., 2003, 278, 30772-30780.

C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor ox” Advan. Enzyme Regul., 2004, 44, 75-92

M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.

S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFA) stabilizers for enhancing erythropoiesis” PCT Int. Appl. (2004), WO 2004108121 A1

C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.

For the expression of EGLN3 see:

R. K. Bruick and S. L. McKnight “A Conserved Family of Prolyl-4-Hydroxylases That Modify HIF” Science, 2001, 294, 1337-1340.

For the expression of HIF2α-CODD see:

a) P. Jaakkola, D. R. Mole, Y.-M. Tian, M. I. Wilson, J. Gielbert, S. J. Gaskell, A. von Kriegsheim, H. F. Hebestreit, M. Mukheiji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.

b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂ Sensing” Science, 2001, 292, 464-468.

For the expression of VHL, elongin b and elongin c see:

A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6HisSumoCysVHL(2-213), 6HisSumoElonginB(1-118), and 6HisSumoElonginC (1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀ for exemplified compounds in the EGLN3 assay ranged from approximately 1-3200 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37deg C./5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

### Measure Epo Protein Produced by Hep3B Cell Line Using AlphaLISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37deg C./5% CO2/90% humidity (standard cell culture incubation conditions). After overnight adherence, medium is removed and replaced with DMEM with 2% serum containing test compound or DMSO negative control. Following 48 hours incubation, cell culture medium is collected and either frozen for later assay or assayed immediately by AlphaLISA to quantitate Epo protein.

The EC₅₀ for exemplar compounds in the Hep3B ELISA AlphaLISA assay ranged from approximately 0.5-100 micromolar, except examples 2, 18, 20, 26, 29, 30, 40, 42, 43, 46, 47, 48, 49, 55, 56, 57, using the reagents and under the conditions outlined herein above. Examples 2, 18, 20, 26, 29, 30, 40, 42, 43, 46, 47, 48, 49, 55, 56, 57, have demonstrated EC₅₀'s in the Hep3B ELISA assay of greater than 100 micromolar, the maximum concentration tested. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permited therapeutic regime.

The foregoing examples and assay have been set forth to illustrate the invention, not limit it. What is reserved to the inventors is to be determined by reference to the claims.

What is claimed is:

1. A compound of formula (I): 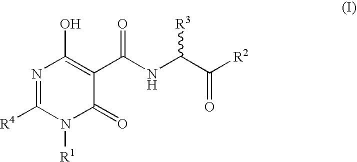
wherein:
R¹ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —NR⁷R⁸ or —OR⁹;
R³ is H or C₁₋C₄alkyl;
R⁴ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀alkyl-C₃-C₈heterocycloalkyl, aryl, C₁₋C₁₀alkyl-aryl, heteroaryl, C₁₋C₁₀alkyl-heteroaryl, —CO(C₁-C₄ alkyl), —CO(C₃-C₆ cycloalkyl), —CO(C₃-C₆ heterocycloalkyl), —CO(aryl), —CO(heteroaryl), and —SO₂(C₁-C₄ alkyl); or R⁵ and R⁶taken together with the nitrogen to which they are attached form a 5- or 6- or 7-membered saturated ring optionally containing one other heteroatom selected from the group consisting of oxygen, nitrogen and sulphur;
R⁷ and R⁸ are each independently selected from the group consisting of hydrogen, C₁₋C₁₀ alkyl, C₂₋C₁₀ alkenyl, C₂₋C₁₀ alkynyl, C₃-C₈ cycloalkyl, C₃-C₈ heterocycloalkyl, aryl and heteroaryl;
R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³, R⁴, R⁵, R⁶, R⁷, R⁸, R⁹ is unsubstituted or, where possible, is substituted with one or more substituents independently selected from the group consisting of C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₀₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt or solvate thereof.

2. A compound according to claim 1 wherein:
R¹ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —OR⁹;
R³ is H or C₁₋C₄alkyl;
R⁴ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³ R⁴, R⁹ is unsubstituted or, where possible, is substituted with one or more substituents independently selected from the group consisting of C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl and heteroaryl, wherein R⁵ and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl; or
a pharmaceutically acceptable salt thereof.

3. A compound according to claim 1 wherein:
R¹ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —OR⁹;
R³ is H or C₁₋C₄alkyl;
R⁴ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁹ is H or a cation;
where any carbon or heteroatom of R¹, R², R³, R⁴ is unsubstituted or, where possible, is substituted with one or more substituents independently selected from C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt thereof.

4. A compound according to claim 1 wherein:
R¹ is hydrogen, C₁₋C₁₀alkyl, C₂₋C₁₀alkenyl, C₂₋C₁₀alkynyl, C₃₋C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —OR⁹;
R³ is H;
R⁴ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁹ is H or a cation;
where any carbon or heteroatom of R¹, R⁴ is unsubstituted or, where possible, is substituted with one or more substituents independently selected from C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —S(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt thereof.

5. A compound according to claim 1 wherein:
R¹ is cyclohexyl, 3-isopropyloxyphenyl, 3-fluorophenyl, 2,3-dichlorophenyl, or 3,5-dichlorophenyl;
R² is OH;
R³ is H; and
R⁴ is cyclohexyl, cycloheptyl, 2-thienyl, or phenyl; or
the pharmaceutically acceptable salts thereof.

6. A compound according to claim 1 which is:
N-{[4-hydroxy-6-oxo-2-phenyl-1-(phenylmethyl)-1,6-dihydro-5-pyrimidinyl]carbonyl}glycine;
N-[(4-hydroxy-6-oxo-2-phenyl-1,6-dihydro-5-pyrimidinyl)carbonyl]glycine;
N-{[4-hydroxy-2-[4-(methyloxy)phenyl]-6-oxo-1-(phenylmethyl)-1,6-dihydro-5-pyrimidinyl]carbonyl}glycine;
N-[(1-{[4-(1,1-dimethylethyl)phenyl]methyl}-4-hydroxy-6-oxo-2-phenyl-1,6-dihydro-5-pyrimidinyl)carbonyl]glycine;
N-{[4-hydroxy-2-(1-methylethyl)-6-oxo-1-(phenylmethyl)-1,6-dihydro-5-pyrimidinyl]carbonyl}glycine;
N-{[2-(2,6-dichlorophenyl)-4-hydroxy-6-oxo-1-(phenylmethyl)-1,6-dihydro-5-pyrimidinyl]carbonyl}glycine;
N-{[4-hydroxy-2-[2-(methyloxy)phenyl]-6-oxo-1-(phenylmethyl)-1,6-dihydro-5-pyrimidinyl]carbonyl}glycine;
N-{[2-(3-bromophenyl)-4-hydroxy-6-oxo-1-(phenylmethyl)-1,6-dihydro-5-pyrimidinyl]carbonyl}glycine;
N-{[2-(3-biphenylyl)-4-hydroxy-6-oxo-1-(phenylmethyl)-1,6-dihydro-5-pyrimidinyl]carbonyl}glycine;
N-{[1-{[4-(1,1-dimethylethyl)phenyl]methyl}-4-hydroxy-6-oxo-2-(phenylamino)-1,6-dihydro-5-pyrimidinyl]carbonyl}glycine;
N-[(2-cyclohexyl-1-{[4-(1,1-dimethylethyl)phenyl]methyl}-4-hydroxy-6-oxo-1,6-dihydro-5-pyrimidinyl)carbonyl]glycine;
N-({1-[(2-chlorophenyl)methyl]-2-cyclohexyl-4-hydroxy-6-oxo-1,6-dihydro-5-pyrimidinyl}carbonyl)glycine;
N-[(2-cyclohexyl-4-hydroxy-6-oxo-1-{[4-(trifluoromethyl)phenyl]methyl}-1,6-dihydro-5-pyrimidinyl)carbonyl]glycine;
N-({1-[(4-bromophenyl)methyl]-2-cyclohexyl-4-hydroxy-6-oxo-1,6-dihydro-5-pyrimidinyl}carbonyl)glycine;
N-({2-cyclohexyl-1-[(3,4-dichlorophenyl)methyl]-4-hydroxy-6-oxo-1,6-dihydro-5-pyrimidinyl}carbonyl)glycine;
N-{[2-[(2,4-difluorophenyl)methyl]-4-hydroxy-6-oxo-1-(phenylmethyl)-1,6-dihydro-5-pyrimidinyl]carbonyl}glycine
N-{[2-[(3,4-difluorophenyl)methyl]-4-hydroxy-6-oxo-1-(phenylmethyl)-1,6-dihydro-5-pyrimidinyl]carbonyl}glycine;
3-(5-{[(carboxymethyl)amino]carbonyl}-4-hydroxy-6-oxo-1,6-dihydro-2-pyrimidinyl)benzoic acid;
N-{[4-hydroxy-1,2-bis(3-methylbutyl)-6-oxo-1,6-dihydro-5-pyrimidinyl]carbonyl}glycine;
N-{[4-hydroxy-6-oxo-1-(phenylmethyl)-2-(3-{[(phenylmethyl)amino]carbonyl}phenyl)-1,6-dihydro-5-pyrimidinyl]carbonyl}glycine;
N-{[4-hydroxy-6-oxo-1,2-bis(phenylmethyl)-1,6-dihydro-5-pyrimidinyl]carbonyl}glycine;
N-({1-[(2-bromophenyl)methyl]-2-[(2-chlorophenyl)methyl]-4-hydroxy-6-oxo-1,6-dihydro-5-pyrimidinyl}carbonyl)glycine;
N-[(4-hydroxy-6-oxo-2-(phenylmethyl)-1-{[4-(4-pyridinyl)phenyl]methyl}-1,6-dihydro-pyrimidinyl)carbonyl]glycine;
N-{[1-[(4-bromophenyl)methyl]-4-hydroxy-6-oxo-2-(phenylmethyl)-1,6-dihydro-5-pyrimidinyl]carbonyl}glycine;
N-{[4-hydroxy-1-{3-[(1-methylethyl)oxy]propyl}-6-oxo-2-(phenylmethyl)-pyrimidinyl]carbonyl}glycine;
3-[5-{[(carboxymethyl)amino]carbonyl}-4-hydroxy-6-oxo-1-(phenylmethyl)-1,6-dihydro-2-pyrimidinyl]benzoic acid;
N-({2-(1,3-benzodioxol-5-ylmethyl)-1-[(2-chlorophenyl)methyl]-4-hydroxy-6-oxo-1,6-dihydro-5-pyrimidinyl}carbonyl)glycine;
N-{[1-(4-biphenylylmethyl)-4-hydroxy-6-oxo-2-(phenylmethyl)-1,6-dihydro-5-pyrimidinyl]carbonyl}glycine;
N-[(2-(2,6-dichlorophenyl)-1-{[4-(1,1-dimethylethyl)phenyl]methyl}-4-hydroxy-6-oxo-1,6-dihydro-5-pyrimidinyl)carbonyl]glycine;
N-{[2-(2-chlorophenyl)-4-hydroxy-6-oxo-1-(phenylmethyl)-1,6-dihydro-5-pyrimidinyl]carbonyl}glycine;
N-{[1-[(2-chlorophenyl)methyl]-2-(2,6-dichlorophenyl)-4-hydroxy-6-oxo-1,6-dihydro-5-pyrimidinyl]carbonyl}glycine;
N-{[2-(2-bromophenyl)-4-hydroxy-6-oxo-1-(phenylmethyl)-1,6-dihydro-5-pyrimidinyl]carbonyl}glycine;
N-{[2-(2,6-dichlorophenyl)-4-hydroxy-6-oxo-1,6-dihydro-5-pyrimidinyl]carbonyl}glycine;
N-[(2-[2,6-bis(methyloxy)phenyl]-1-{[4-(1,1-dimethylethyl)phenyl]methyl}-4-hydroxy-6-oxo-1,6-dihydro-5-pyrimidinyl)carbonyl]glycine;
N-{[1-cyclohexyl-2-(2,6-dichlorophenyl)-4-hydroxy-6-oxo-1,6-dihydro-5-pyrimidinyl]carbonyl}glycine;
N-{[2-(²-biphenylyl)-4-hydroxy-6-oxo-1-(phenylmethyl)-1,6-dihydro-5-pyrimidinyl]carbonyl}glycine;
N-{[1-(2-cyclopropylethyl)-2-(2,6-dichlorophenyl)-4-hydroxy-6-oxo-1,6-dihydro-5-pyrimidinyl]carbonyl}glycine;
N-[(2-[2,6-dichloro-4-(trifluoromethyl)phenyl]-1-{[4-(1,1-dimethylethyl)phenyl]methyl}-4-hydroxy-6-oxo-1,6-dihydro-5-pyrimidinyl)carbonyl]glycine;
N-{[1-(4-biphenylylmethyl)-2-(2,6-dichlorophenyl)-4-hydroxy-6-oxo-1,6-dihydro-5-pyrimidinyl]carbonyl}glycine;
N-{[1-{[4-(1,1-dimethylethyl)phenyl]methyl}-2-(2,6-dimethylphenyl)-4-hydroxy-6-oxo-1,6-dihydro-5-pyrimidinyl]carbonyl}glycine;
N-[(2-{2,6-bis[(2,2,2-trifluoroethyl)oxy]phenyl}-1-{[4-(1,1-dimethylethyl)phenyl]methyl}-4-hydroxy-6-oxo-1,6-dihydro-5-pyrimidinyl)carbonyl]glycine;
N-[(2-(2,6-dibromophenyl)-1-{[4-(1,1-dimethylethyl)phenyl]methyl}-4-hydroxy-6-oxo-1,6-dihydro-5-pyrimidinyl)carbonyl]glycine;
N-{[1-{[4-(1,1-dimethylethyl)phenyl]methyl}-4-hydroxy-6-oxo-2-(1,1′:3′,1″-terpenyl-2′-yl)-1,6-dihydro-5-pyrimidinyl]carbonyl}glycine;
N-[(2-(2-bromophenyl)-1-{[4-(1,1-dimethylethyl)phenyl]methyl}-4-hydroxy-6-oxo-1,6-dihydro-5-pyrimidinyl)carbonyl]glycine;
N-[(2-[4′-(1,1-dimethylethyl)-2-biphenylyl]-1-{[4-(1,1-dimethylethyl)phenyl]methyl}-4-hydroxy-6-oxo-1,6-dihydro-5-pyrimidinyl)carbonyl]glycine;
N-[(2-(2-biphenylyl)-1-{[4-(1,1-dimethylethyl)phenyl]methyl}-4-hydroxy-6-oxo-1,6-dihydro-5-pyrimidinyl)carbonyl]glycine;
N-[(2-(3′,5′-difluoro-2-biphenylyl)-1-{[4-(1,1-dimethylethyl)phenyl]methyl}-4-hydroxy-6-oxo-1,6-dihydro-5-pyrimidinyl)carbonyl]glycine;
N-({1-{[4-(1,1-dimethylethyl)phenyl]methyl}-4-hydroxy-2-[3-methyl-1-(2-methylpropyl)butyl]-6-oxo-1,6-dihydro-5-pyrimidinyl}carbonyl)glycine;
N-({1-{[4-(1,1-dimethylethyl)phenyl]methyl}-4-hydroxy-6-oxo-2-[4′-(triflouromethyl)-2-biphenylyl]-1,6-dihydro-5-pyrimidinyl}carbonyl)glycine;
N-[(2-(2,3-dichlorophenyl)-1-{[4-(1,1-dimethylethyl)phenyl]methyl}-4-hydroxy-6-oxo-1,6-dihydro-5-pyrimidinyl)carbonyl]glycine;
N-[(2-(2,5-dichlorophenyl)-1-{[4-(1,1-dimethylethyl)phenyl]methyl}-4-hydroxy-6-oxo-1,6-dihydro-5-pyrimidinyl)carbonyl]glycine;
N-[(2-cyclopentyl-1-{[4-(1,1-dimethylethyl)phenyl]methyl}-4-hydroxy-6-oxo-1,6-dihydro-5-pyrimidinyl)carbonyl]glycine;
N-[(2-(cyclopropylmethyl)-1-{[4-(1,1-dimethylethyl)phenyl]methyl}-4-hydroxy-6-oxo-1,6-dihydro-5-pyrimidinyl)carbonyl]glycine;
N-[(2-cycloheptyl-1-{[4-(1,1-dimethylethyl)phenyl]methyl}-4-hydroxy-6-oxo-1,6-dihydro-5-pyrimidinyl)carbonyl]glycine;
N-[(2-(3-chloro-2-biphenylyl)-1-{[4-(1,1-dimethylethyl)phenyl]methyl}-4-hydroxy-6-oxo-1,6-dihydro-5-pyrimidinyl)carbonyl]glycine;
N-({2-(3-chloro-2-biphenylyl)-1-[(2-chlorophenyl)methyl]-4-hydroxy-6-oxo-1,6-dihydro-5-pyrimidinyl}carbonyl)glycine;
N-{[1-{[4-(1,1-dimethylethyl)phenyl]methyl}-4-hydroxy-6-oxo-2-(2,4,6-trichlorophenyl)-1,6-dihydro-5-pyrimidinyl]carbonyl}glycine;
N-{[1-[(2-chlorophenyl)methyl]-2-(2,6-dibromophenyl)-4-hydroxy-6-oxo-1,6-dihydro-5-pyrimidinyl]carbonyl}glycine;
N-[(2-[1-(4-chlorophenyl)cyclopropyl]-1-{[4-(1,1-dimethylethyl)phenyl]methyl}-4-hydroxy-6-oxo-1,6-dihydro-5-pyrimidinyl)carbonyl]glycine;
N-[(2-(2,6-difluorophenyl)-1-{[4-(1,1-dimethylethyl)phenyl]methyl}-4-hydroxy-6-oxo-1,6-dihydro-5-pyrimidinyl)carbonyl]glycine;
N-{[1-cyclohexyl-2-(2,6-dibromophenyl)-4-hydroxy-6-oxo-1,6-dihydro-5-pyrimidinyl]carbonyl}glycine;
N-{[1-{[4-(1,1-dimethylethyl)phenyl]methyl}-4-hydroxy-6-oxo-2-(1-phenylcyclopentyl)-1,6-dihydro-5-pyrimidinyl]carbonyl}glycine;
N-{[1-{[4-(1,1-dimethylethyl)phenyl]methyl}-4-hydroxy-6-oxo-2-(2,3,5,6-tetrachlorophenyl)-1,6-dihydro-5-pyrimidinyl]carbonyl}glycine;
N-{[1-{[4-(1,1-dimethylethyl)phenyl]methyl}-4-hydroxy-6-oxo-2-(3-thienyl)-1,6-dihydro-5-pyrimidinyl]carbonyl}glycine;
N-{[1-(1-ethylpropyl)-4-hydroxy-6-oxo-2-phenyl-1,6-dihydro-5-pyrimidinyl]carbonyl}glycine;
N-[(2-[bicyclo[2.2.1]hept-2-yl]-1-{[4-(1,1-dimethylethyl)phenyl]methyl}-4-hydroxy-6-oxo-1,6-dihydro-5-pyrimidinyl)carbonyl]glycine;
N-[(1,2-dicyclohexyl-4-hydroxy-6-oxo-1,6-dihydro-5-pyrimidinyl)carbonyl]glycine;
N-[(2-cycloheptyl-1-cyclohexyl-4-hydroxy-6-oxo-1,6-dihydro-5-pyrimidinyl)carbonyl]glycine;
N-{[1-{[4-(1,1-dimethylethyl)phenyl]methyl}-4-hydroxy-6-oxo-2-(4-pyridinyl)-1,6-dihydro-5-pyrimidinyl]carbonyl}glycine;
N-({1-[(2-chlorophenyl)methyl]-4-hydroxy-6-oxo-2-phenyl-1,6-dihydro-5-pyrimidinyl}carbonyl)glycine;
N-{[1-cyclohexyl-4-hydroxy-6-oxo-2-(3-thienyl)-1,6-dihydro-5-pyrimidinyl]carbonyl}glycine;
N-[(1-cyclohexyl-4-hydroxy-6-oxo-2-phenyl-1,6-dihydro-5-pyrimidinyl)carbonyl]glycine
N-{[1-{[4-(1,1-dimethylethyl)phenyl]methyl}-4-hydroxy-6-oxo-2-(2-thienyl)-1,6-dihydro-5-pyrimidinyl]carbonyl}glycine;
N-{[2-cyclohexyl-1-(4-fluorophenyl)-4-hydroxy-6-oxo-1,6-dihydro-5-pyrimidinyl]carbonyl}glycine;
N-{[1-(2-chlorophenyl)-2-cyclohexyl-4-hydroxy-6-oxo-1,6-dihydro-5-pyrimidinyl]carbonyl}glycine;
N-[(2-cyclohexyl-4-hydroxy-6-oxo-1-phenyl-1,6-dihydro-5-pyrimidinyl)carbonyl]glycine;
N-{[2-cyclohexyl-1-(2,6-dichlorophenyl)-4-hydroxy-6-oxo-1,6-dihydro-5-pyrimidinyl]carbonyl}glycine;
N-[(1-{[4-(1,1-dimethylethyl)phenyl]methyl}-4-hydroxy-6-oxo-1,6-dihydro-2,2′-bipyrimidin-5-yl)carbonyl]glycine;
N-[(2-(3,5-dichloro-4-pyridinyl)-1-{[4-(1,1-dimethylethyl)phenyl]methyl}-4-hydroxy-6-oxo-1,6-dihydro-5-pyrimidinyl)carbonyl]glycine;
N-({2-cyclohexyl-1-[4-(1,1-dimethylethyl)phenyl]-4-hydroxy-6-oxo-1,6-dihydro-5-pyrimidinyl}carbonyl)glycine;
N-{[2-cyclohexyl-1-(2-fluorophenyl)-4-hydroxy-6-oxo-1,6-dihydro-5-pyrimidinyl]carbonyl}glycine;
N-{[1-(3-bromophenyl)-2-cyclohexyl-4-hydroxy-6-oxo-1,6-dihydro-5-pyrimidinyl]carbonyl}glycine;
N-{[2-cyclohexyl-1-(3-fluorophenyl)-4-hydroxy-6-oxo-1,6-dihydro-5-pyrimidinyl]carbonyl}glycine;
N-( {1-cyclohexyl-2-[cyclohexyl(phenyl)methyl]-4-hydroxy-6-oxo-1,6-dihydro-5-pyrimidinyl}carbonyl)glycine;
N-{[1-cyclohexyl-2-(diphenylmethyl)-4-hydroxy-6-oxo-1,6-dihydro-5-pyrimidinyl]carbonyl}glycine;
N-[(2-cyclohexyl-4-hydroxy-1-{3-[(1-methylethyl)oxy]phenyl}-6-oxo-1,6-dihydro-5-pyrimidinyl)carbonyl]glycine;
N-{[-(5-bromo-2-chlorophenyl)-2-cyclohexyl-4-hydroxy-6-oxo-1,6-dihydro-5-pyrimidinyl]carbonyl}glycine;
N-{[2-cyclohexyl-1-(2,3-dichlorophenyl)-4-hydroxy-6-oxo-1,6-dihydro-5-pyrimidinyl]carbonyl}glycine;
N-[(1-cyclohexyl-2-ethyl-4-hydroxy-6-oxo-1,6-dihydro-5-pyrimidinyl)carbonyl]glycine
N-[(1-{[4-(1,1-dimethylethyl)phenyl]methyl}-2-ethyl-4-hydroxy-6-oxo-1,6-dihydro-5-pyrimidinyl)carbonyl]glycine;
N-{[2-cyclohexyl-1-(3,5-difluorophenyl)-4-hydroxy-6-oxo-1,6-dihydro-5-pyrimidinyl]carbonyl}glycine;
N-{[2-cyclohexyl-1-(3,5-dichlorophenyl)-4-hydroxy-6-oxo-1,6-dihydro-5-pyrimidinyl]carbonyl}glycine;
N-[(1-{[4-(1,1-dimethylethyl)phenyl]methyl}-4-hydroxy-2-methyl-6-oxo-1,6-dihydro-5-pyrimidinyl)carbonyl]glycine;
N-[(4-hydroxy-6-oxo-1,2-diphenyl-1,6-dihydro-5-pyrimidinyl)carbonyl]glycine;
N-{[2-cyclohexyl-1-(2,4-dichlorophenyl)-4-hydroxy-6-oxo-1,6-dihydro-5-pyrimidinyl]carbonyl}glycine;
N-({2-cyclohexyl-4-hydroxy-6-oxo-1-[3-(phenyloxy)phenyl]-1,6-dihydro-5-pyrimidinyl}carbonyl)glycine;
N-[(1-cyclohexyl-4-hydroxy-2-methyl-6-oxo-1,6-dihydro-5-pyrimidinyl)carbonyl]glycine;
N-[(²-cyclohexyl-4-hydroxy-1-{4-[(1-methylethyl)oxy]phenyl}-6-oxo-1,6-dihydro-5-pyrimidinyl)carbonyl]glycine;
N-{[2-cyclohexyl-1-(2,3-difluorophenyl)-4-hydroxy-6-oxo-1,6-dihydro-5-pyrimidinyl]carbonyl}glycine;
N-{[2-cyclohexyl-4-hydroxy-1-(4-iodophenyl)-6-oxo-1,6-dihydro-5-pyrimidinyl]carbonyl}glycine;
N-{[2-cyclohexyl-1-(1-ethylpropyl)-4-hydroxy-6-oxo-1,6-dihydro-5-pyrimidinyl]carbonyl}glycine;
or a pharmaceutically acceptable salt or solvate thereof.

7. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

8. A pharmaceutical composition comprising a compound of formula (I) or a salt, solvate, according to claim 1 and one or more of pharmaceutically acceptable carriers, diluents and excipients.

9. The use of a compound according to claim 1 for treating anemia in a mammal, which method comprises administering an effective amount of a compound of formula (I) or a salt or solvate thereof according to claim 1, neat or admixed with a pharmaceutically acceptable carrier, to a mammal suffering from anemia which can be treated by inhibiting HIF prolyl hydroxylases.

10. A process for preparing a compound of formula (I) 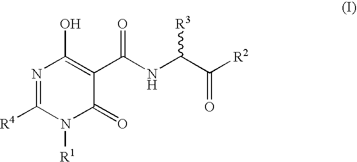
wherein:
R¹ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —NR⁷R⁸ or —OR⁹;
R³ is H or C₁₋C₄alkyl;
R⁴ is hydrogen, —NR⁵R⁶, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁵ and R⁶ are each independently selected from the group consisting of hydrogen, C₁₋C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl, —CO(C₁-C₄ alkyl), —CO(C₃-C₆ cycloalkyl), —CO(C₃—C₆ heterocycloalkyl), —CO(aryl), —CO(heteroaryl), and —SO₂(C₁-C₄ alkyl); or R⁵ and R⁶taken together with the nitrogen to which they are attached form a 5- or 6- or 7-membered saturated ring optionally containing one other heteroatom selected from the group consisting of oxygen, nitrogen and sulphur;
R⁷ and R⁸ are each independently selected from the group consisting of hydrogen, C₁₋C₁₀ alkyl, C₂C₁₀ alkenyl, C₂₋C₁₀ alkynyl, C₃-C₈ cycloalkyl, C₃-C₈ heterocycloalkyl, aryl and heteroaryl;
R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R², R³, R⁴, R⁵, R⁶l R⁷, R⁸, R⁹ is unsubstituted or, where possible, is substituted with one or more substituents independently selected from the group consisting of C₁-C₆ alkyl, aryl, heteroaryl, halogen, —OR¹⁰, —NR⁵R⁶, cyano, nitro, —C(O)R¹⁰, —C(O)OR¹⁰, —SR¹⁰, —(O)R¹⁰, —S(O)₂R¹⁰, —NR⁵R⁶, —CONR⁵R⁶, —N(R⁵)C(O)R¹⁰, —N(R⁵)C(O)OR¹⁰, —OC(O)NR⁵R⁶, —N(R⁵)C(O)NR⁵R⁶, —SO₂NR⁵R⁶, —N(R⁵)SO₂R¹⁰, C₁-C₁₀, alkenyl, C₁-C₁₀ alkynyl, C₃-C₆ cycloalkyl, C₃-C₆ heterocycloalkyl, aryl or heteroaryl group, wherein R⁵, and R⁶ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
comprising treating a compound of formula A: 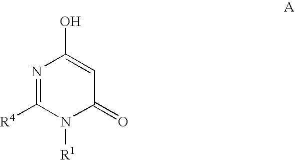
wherein R¹ and R⁴ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (B) wherein R¹, R³ and R⁴ are the same as for those groups in formula (I); 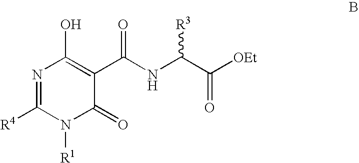
and treating the compound of formula (B) with an alkali such as sodium hydroxide, in an appropriate solvent, such as aqueous ethanol, at a suitable temperature such as room temperature, to form a compound of formula (I) where R² is —OH.
